SEQUENCE LISTING (1 sequence)

SEQ ID NO: 1 - protein, 8 aa, Artificial
  synthetic peptide
SIINFEKL

(19) United States
(12) Patent Application Publication
(10) Pub. No.: US 2007/0243575 A1
(43) Pub. Date: Oct. 18, 2007

(54) Compositions and methods for treatment of immune-related disease

(75) Inventors: Kasper Hoebe, San Diego, CA (US); Bruce Beutler, San Diego, CA (US); Koichi Tabeta, (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA (US)

(21) Appl. No.: 11/702,869

(22) Filed: Feb. 6, 2007

Related U.S. Application Data
(60) Provisional application No. 60/765,572, filed on Feb. 6, 2006.

(51) Int. Cl. C12Q 1/02

(52) U.S. Cl. 435/29

(57) ABSTRACT

The invention provides methods for identifying novel agents for modulating the immune system or compounds useful for modulating immune responses. Also provided are compositions and methods for preventing or treating immune-related diseases, infectious diseases, or neoplastic diseases in mammalian subjects. Pharmaceutical compositions are provided for treating such diseases.

### CROSS-REFERENCE TO RELATED APPLICATIONS

The subject patent application claims the benefit of priority to U.S. Provisional Patent Application No. 60/765,572 (filed Feb. 6, 2006). The full disclosures of the priority application are incorporated herein by reference in their entirety and for all purposes.

### STATEMENT OF GOVERNMENT SUPPORT

This invention was made in part by government support by The National Institutes of Health Grant No. U54 AI054523. The Government has certain rights in this invention.

### FIELD OF THE INVENTION

This invention generally relates to compositions and methods for preventing or treating a immune-related disease, an infectious disease, or a neoplastic disease in a mammalian subject and a pharmaceutical composition for treating a immune-related disease in the mammalian subject. The invention also pertains to methods of screening for novel agents that are useful in these therapeutic applications.

### BACKGROUND

It is commonly thought that apoptosis is a bland process that does not stimulate innate or adaptive immune responses. Gallucci et al., Nat. Med. 5: 1249-1255, 1999; Blander et al., Science 304: 1014-1018, 2004; Li et al., J. Immunol. 166: 7128-7135, 2001; Steinman et al., J. Exp. Med. 191: 411-416, 2000. Yet programmed cell death is induced by a variety of viral infections, and teleological reasoning suggests that the host would benefit from exercise of a mechanism serving the immune detection of foreign proteins expressed in the context of a dying cell; an idea that has previously been posed as the danger hypothesis. Matzinger, Annu. Rev. Immunol. 12: 991-1045, 1994. Indeed, evidence suggest that apoptosis contributes to the onset of an adaptive immune response either directly or in the context of viral and/or bacterial infections. Albert et al., Nature 392: 86-89, 1998; Finberg et al., J. Immunol. 123: 1205-1209, 1979; Ronchetti et al., J. Immunol. 163: 130-136, 1999; Gorla et al, AIDS Res. Hum. Retroviruses 10: 1097-1103, 1994; Yrlid et al., J. Exp. Med. 191: 613-624, 2000. However, the underlying mechanisms for these observations remain elusive. TLRs are well recognized for their ability to sense molecules of microbial origin and to mediate microbial adjuvanticity. Akira et al., Nat. Rev. Immunol. 4: 499-511, 2004; Iwasaki et al., Nat. Immunol. 5: 987-995, 2004; Hoebe et al., Nat. Immunol. 4: 1223-1229, 2003; Schnare et al., Nat. Immunol. 2: 947-950, 2001. It has been suggested that the promiscuity of TLR receptors permits activation not only by molecules derived from microbes, but also by endogenous molecules that may possess similar chemical properties. Seong et al., Nat. Rev. Immunol. 4: 469-478, 2004

### SUMMARY

In one aspect, the invention provides methods for identifying modulators of the immune system or compounds which are useful for modulating immune responses. These methods involve first contacting test compounds with apoptotic cell-responding B220⁻ dendritic cells in the presence of an apoptotic cell, and then detecting a change of a signaling activity of the apoptotic cell-responding B220⁻ dendritic cell relative to its signaling activity in the absence of test compounds. Compounds which cause a change (increase or decrease) of the signaling activity of the dendritic cells can be useful for modulating innate or antigen-specific immune responses.

Some of these methods employ apoptotic cell-responding B220⁻ dendritic cells that are Flt3L-induced bone marrow-derived B220⁻ dendritic cells or spleen derived CD11b-/CD11c+B220⁻ dendritic cells. In some methods, the apoptotic cell is a splenocyte treated with γ-irradiation, UV-irradiation or Fas-activating antibody. Some of the methods are directed to identifying agents which cause an increased signaling activity of the dendritic cells. Some other methods are directed to identifying agents which cause a decreased signaling activity of the dendritic cells. In some of the methods, the apoptotic cell-responding B220⁻ dendritic cells are contacted with the test compounds prior to stimulation with the apoptotic cell. In other methods, the apoptotic cell-responding B220⁻ dendritic cells are contacted with the test compounds subsequent to or simultaneously with stimulation with the apoptotic cell.

In some screening methods of the invention, the monitored signaling activity of the dendritic cells is production of type I interferon by the dendritic cells upon stimulation with the apoptotic cell. In some other methods, the monitored signaling activity is uptake by the dendritic cells of apoptotic material (e.g., an antigen) from the apoptotic cell. In these methods, the apoptotic cell can be a UV-treated splenocyte that is labeled with a fluorescent marker. Uptake of apoptotic material by the dendritic cells in these methods can be quantitated, e.g., by FACS. In still some other methods, the monitored signaling activity of the dendritic cells is activation of a T cell proliferation. For example, activity of the dendritic cells to crossprime a CD8⁺ T cell or a CD4⁺ T cell can be examined in these methods. The T cell used in these methods can be fluorescently labeled. Proliferation of the T cell can be examined by, e.g., FACS.

In a related aspect, the invention provides methods for identifying agents useful for modulating an antigen specific adaptive immune response. These methods entail first contacting a test compound with an apoptotic cell-responding B220⁻ dendritic cell in the presence of an apoptotic cell which presents the antigen, and then detecting a change of a signaling activity of the apoptotic cell-responding B220⁻ dendritic cell relative to its signaling activity in the absence of the test compound. Compounds thus identified are capable of modulating adaptive immune responses specific for the antigen. Some of the methods employ an apoptotic cell-responding B220⁻ dendritic cell that is a FIt3L-induced bone marrow-derived B220⁻ dendritic cell or a spleen derived CD11b⁻/CD11c⁺ B220⁻ dendritic cell. The apoptotic cell used in these methods can be a splenocyte treated with γ-irradiation, UV-irradiation or Fas-activating antibody. In some of the methods, the antigen presented by the apoptotic cell is a bacterial or a viral antigen. Compounds are identified in these methods which can enhance the signaling activity of the dendritic cell. In some other methods, the apoptotic cell presents an autoantigen or an allogenic graft antigen. Compounds are identified which can inhibit the signaling activity of the dendritic cell. Examples of signaling activities of the dendritic cells that can be monitored in these screening methods include production of type I interferon, uptake apoptotic material from the apoptotic cell and activation of a T cell proliferation.

In another aspect, the invention provides methods for identifying agents useful for modulating innate immunity. These methods involve contacting test compounds with apoptotic cell-responding B220⁻ dendritic cells in the presence of an apoptotic cell, and then detecting a change of a signaling activity of the apoptotic cell-responding B220⁻ dendritic cells relative to their signaling activity in the absence of the test compounds. This allows identification of modulators of innate immune responses. Some of the methods employ an apoptotic cell-responding B220⁻ dendritic cell that is an FIt3L-induced bone marrow-derived B220⁻ dendritic cell or a spleen derived CD11b⁻/CD11c⁺ B220⁻ dendritic cell. The apoptotic cell used in these methods can be a splenocyte treated with γ-irradiation, UV-irradiation or Fas-activating antibody. Some of the methods are directed to identifying compounds useful for enhancing the signaling activity of the dendritic cell and thereby stimulating innate immunity. Some of the methods are directed to identifying compounds useful for inhibiting the signaling activity of the dendritic cell and thereby suppressing innate immunity. Signaling activities of the dendritic cells that can be monitored in these screening methods include, e.g., production of type I interferon, uptake apoptotic material from the apoptotic cell and activation of a T cell proliferation.

In one aspect, the invention provides methods for preventing or treating disease in a mammalian subject. A method for preventing or treating immune-related disease in a mammalian subject is provided comprising administering to the mammalian subject an antagonist of apoptotic cell-responding B220⁻ dendritic cell (DC) and reducing a cytotoxic T cell response in the mammalian subject wherein the disease is prevented or treated. Diseases subject to treatment include, but are not limited to, autoimmune disease or allogeneic tissue rejection. A method for preventing or treating a disease in a mammalian subject is provided comprising administering to the mammalian subject an agonist of apoptotic cell-responding B220⁻ dendritic cell, and activating a cytotoxic T cell response in the mammalian subject wherein the disease is prevented or treated. Diseases subject to treatment include, but are not limited to, infectious disease or neoplastic disease. Various applications of the compositions and methods are envisioned. The antagonist of apoptotic cell-responding B220 dendritic cell can act by interfering with an apoptotic cell-derived ligand recognition by B220 negative dendritic cells in the mammalian subject and further reducing a cytotoxic T cell response in the mammalian subject wherein the disease is prevented or treated. The agonist of apoptotic cell-responding B220⁻ dendritic cell can act by potentiating an apoptotic cell-derived ligand recognition by B220 negative dendritic cells in the mammalian subject and further activating a cytotoxic T cell response in the mammalian subject wherein the disease is prevented or treated.

In one aspect of the composition and method of the present invention, a TLR-independent pathway for adaptive immune activation is described. This pathway senses antigens expressed by cells undergoing programmed death, and depends upon specific proteins that contribute to innate immune responses. The pathway is represented within a specific subset of FIt3L-induced bone marrow-derived dendritic cells (BMDC) or within CD11c⁺/MHC-II⁺/CD11b⁻/CD8⁻/B220⁻ DCs isolated from spleen cells.

A method for preventing or treating an autoimmune disease in a mammalian subject is provided comprising administering to the mammalian subject an antagonist of apoptotic cell-responding B220⁻ dendritic cell, and reducing a cytotoxic T cell response in the mammalian subject wherein the autoimmune disease is prevented or treated. The method can further comprise reducing type I interferon in blood cells or tissue of the mammalian subject. In one aspect, the cytotoxic T cell response is a CD8+ T cell response. In a detailed aspect, the antagonist is a polypeptide, nucleic acid, small molecule, antisense oligonucleotide, ribozyme, RNAi construct, siRNA, shRNA, or antibody. The autoimmune disease includes, but is not limited to, systemic lupus erythematosus, rheumatoid arthritis, multiple sclerosis, diabetes, inflammatory bowel disease, psoriasis, or asthma.

A method for treating allogeneic tissue rejection in a mammalian subject is provided comprising administering to the mammalian subject an antagonist of apoptotic cell-responding B220⁻ dendritic cell, and reducing a cytotoxic T cell response in the mammalian subject wherein the allogeneic tissue rejection is treated. The method can further comprise reducing type I interferon in blood cells or tissue of the mammalian subject. In one aspect, the cytotoxic T cell response is a CD8⁺ T cell response. In a detailed aspect, the antagonist is a polypeptide, nucleic acid, small molecule, antisense oligonucleotide, ribozyme, RNAi construct, siRNA, shRNA, or antibody.

A method for preventing or treating infectious disease in a mammalian subject is provided comprising administering to the mammalian subject an agonist of apoptotic cell-responding B220⁻ dendritic cell, and activating a cytotoxic T cell response in the mammalian subject wherein the infectious disease is prevented or treated. The method can further comprise increasing type I interferon in blood cells or tissue of the mammalian subject. In one aspect, the cytotoxic T cell response is a CD8⁺ T cell response. In a detailed aspect, the antagonist is a polypeptide, nucleic acid, small molecule, antisense oligonucleotide, ribozyme, RNAi construct, siRNA, shRNA, or antibody. In a detailed aspect, the infectious disease is a viral disease, a bacterial disease or a parasitic disease.

A method for treating neoplastic disease in a mammalian subject is provided comprising administering to the mammalian subject an agonist of apoptotic cell-responding B220⁻ dendritic cell, and activating a cytotoxic T cell response in the mammalian subject wherein the neoplastic disease is treated. The method can further comprise increasing type I interferon in blood cells or tissue of the mammalian subject. In one aspect, the cytotoxic T cell response is a CD8⁺ T cell response. In a detailed aspect, the agonist is a polypeptide, nucleic acid, small molecule, antisense oligonucleotide, ribozyme, RNAi construct, siRNA, shRNA, or antibody. The neoplastic disease includes, but is not limited to, cancer, solid tumor, sarcoma, melanoma, carcinoma, leukemia, or lymphoma.

A method for treating sepsis in a mammalian subject is provided comprising administering to the mammalian subject an agonist of apoptotic cell-responding B220⁻ dendritic cell, and activating a cytotoxic T cell response in the mammalian subject wherein sepsis is treated. The method can further comprise increasing type I interferon in blood cells or tissue of the mammalian subject. In one aspect, the cytotoxic T cell response is a CD8⁺ T cell response. In a detailed aspect, the agonist is a polypeptide, nucleic acid, small molecule, antisense oligonucleotide, ribozyme, RNAi construct, siRNA, shRNA, or antibody.

A method for stimulating a CD8⁺ T cell response in a mammalian subject is provided comprising administering to the mammalian subject an agonist of apoptotic cell-responding B220⁻ dendritic cell, and activating the CD8⁺ T cell response in the mammalian subject. The method can further comprise increasing type I interferon in blood cells or tissue of the mammalian subject. In a detailed aspect, the antagonist is a polypeptide, nucleic acid, small molecule, antisense oligonucleotide, ribozyme, RNAi construct, siRNA, shRNA, or antibody.

A method for depleting apoptotic cells in a mammalian subject is provided comprising administering to the mammalian subject an agonist of B220 negative dendritic cell recognition of the apoptotic cells, and activating a cytotoxic T cell response in the mammalian subject thereby removing the apoptotic cells in the mammalian subject.

### BRIEF DESCRIPTION OF THE DRAWINGS

FIGS. 1a and 1b show that percentage of cells undergoing apoptosis as measured by annexin V and propidium iodide in isolated splenocytes.

FIGS. 2a, 2b, 2c, 2d, 2e, and 2f show that apoptotic but not live act-mOVA cells induce strong CTL responses.

FIGS. 3a, 3b, 3c, 3d, 3e, 3f, 3g, and 3h show that type I IFN is a key mediator of apoptotic cell-induced CTL responses and is induced via a TLR-independent pathway.

FIGS. 4a, 4b, 4c, 4d, 4e, 4f, 4g, and 4h show that abrogated TLR signaling in MyD88^{-/-}; Trif^{lps2/lps2} double-deficient mice.

FIGS. 5a, 5b, and 5c show that flowcytometric and functional characterization of B220⁺ and B220⁻ Flt3L- and GM-CSF-derived dendritic cells from bone marrow.

FIGS. 6a, 6b, 6c, 6d, 6e, and 6f show that FIt3L- and GM-CSF-derived BMDCs respond differently to apoptotic cells.

FIGS. 7a, 7b, 7c, and 7d show that FIt3L-derived B220⁻ DCs efficiently (cross)-prime CD8⁺ and CD4⁺ T cells and can serve as bystander cells.

FIG. 8 shows that control incubations for (cross-)priming of OT I/II cells by the different DC subsets. Note that Flt3L-derived B220⁻ DCs cannot serve as bystander cells when direct contact with apoptotic cells is omitted by using transwell membrane inserts.

FIGS. 9a, 9b, and 9c show that expression of CD40, CD80 and CD86 costimulatory molecules on GM-CSF and Flt3L-derived B220⁻ BMDCs after 24 hr incubation with or without apoptotic cells.

FIGS. 10a, 10b, 10c, 10d, 10e, 10f, and 10g show that CD36 and UNC-93B are essential in apoptotic cell-induced immune responses.

FIGS. 11a, 11b, and 11c show that DC subsets derived from Unc931^{3d/3d} mutant mice show normal surface expression of MHC class I/II and costimulatory molecule expression as well as normal uptake of apoptotic cells.

### DETAILED DESCRIPTION

The present invention is predicated in part on the discoveries of a previously unrecognized pathway for activation of immune responses which is directed toward detection of antigens expressed by apoptotic cells. It was found that the innate and subsequent adaptive immune response triggered by apoptotic cells in this pathway is independent of Toll-Interleukin I Receptor signaling. Specifically, the present inventors discovered that a subset of Flt-3 Ligand-derived dendritic cells (DCs) when exposed to apoptotic cells were able to produce type I interferon and favored the development of cytotoxic T cell responses. These apoptotic cell-responding dendritic cells can also be isolated from spleen. In vitro, these dendritic cells purified from borrow marrow or spleen can “sample” apoptotic cells through “nibbling” and stimulate proliferation of T cells (e.g., crosspriming CD8⁺ T cells).

As detailed in the Examples, the TLR-independent pathway senses antigens expressed by cells undergoing programmed death, and depends upon specific proteins that contribute to innate immune responses. This pathway for activation of antigen-specific adaptive immune responses is represented within a specific subset of Flt-3 Ligand (FIt3L)-induced bone marrow-derived dendritic cells (BMDCs) or within CD11c⁺/MHC-II⁺/CD11b⁻/CD8⁻/B220⁻ specific DCs isolated from spleen cells. The pathway does not involve GM-CSF-treated bone marrow-derived dendritic cells. Exposure of DCs to apoptotic cells results in production of type I interferon, and favors the development of cytotoxic T cell responses. The ENU-induced germline mutation 3d(Unc3b1^{3d/3d}) abolishes both class I and class II responses elicited by this pathway, while a null allele of Cd36 (Cd₃₆^{obl/obl}) selectively abolishes class II responses. It is proposed that this mode of adaptive immune activation evolved to permit the sensitive detection of intracellular microbial infections (particularly viral infections, which frequently induce apoptotic cell death), but may also be important in transplantation, autoimmunity, and vaccine development.

In accordance with these discoveries, the present invention provides methods of identifying compounds to identify agents which can be used to modulate (e.g., inhibit or stimulate) immune responses. These methods utilize the apoptotic cell-responding dendritic cells described herein and screen for compounds which can modulate a signaling activity of the dendritic cells when they are stimulate with apoptotic cells. Some of the methods are used to identify agents which can modulate an antigen-specific immune responses. In these methods, apoptotic cells which present the specific antigen (e.g., a bacterial or viral antigen) is employed. Some of these methods are used to identify agents which can modulate innate immunity. In such methods, the apoptotic cells do not present any specific antigen. Compounds identified with these methods are used to modulate innate immune responses (e.g., as adjuvant in vaccination).

The invention also provides methods for preventing or treating disease in a mammalian subject. Such methods comprise administering to the mammalian subject an agonist or an antagonist of apoptotic cell-responding B220⁻ dendritic cell, and altering a cytotoxic T cell response in the mammalian subject wherein the disease is prevented or treated. A cytotoxic T cell response can be reduced by an antagonist of the signaling pathway, or a cytotoxic T cell response can be increased by an agonist of the signaling pathway. The disease state can include, but is not limited to, immune related diseases such as autoimmune disease or allogeneic tissue rejection, infectious disease, and neoplastic disease.

It is to be understood that this invention is not limited to particular methods, reagents, compounds, compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural references unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“B220 negative dendritic cells” refer to dendritic cells that lack the B220 marker, but may be positive or negative for other cell markers. “Apoptotic cell-responding B220⁻ dendritic cells” or “B220 negative dendritic cell recognition of apoptotic cells” refer to B220 negative dendritic cells which are capable of activating antigen-specific adaptive immune responses when stimulated by apoptotic cells, as described in the Examples below. Unless otherwise noted, “apoptotic cell-responding dendritic cell” is used herein interchangeably with “apoptotic cell-responding B220⁻ dendritic cell.” Examples of apoptotic cell-responding B220⁻ dendritic cells include Flt-3 Ligand (FIt3L)-induced bone marrow-derived dendritic cells (BMDCs) or CD11c⁺/MHC-II⁺/CD11b⁻/B220⁻ specific DCs (CD8⁻or CD8⁺) isolated from spleen cells. They do not encompass GM-CSF-treated bone marrow-derived dendritic cells. Methods for isolating apoptotic cell-responding B220⁻ dendritic cells and assaying their signaling activities are described in detail in the Examples below.

“Signaling activity” of apoptotic cell-responding B220⁻ dendritic cells refers to any activity of these dendritic cells that is associated with their function in mediating T cell activation and proliferation, and subsequent innate or antigen-specific immune responses as described herein. Examples of such signaling activities include production of type I interferon in response to stimulation of apoptotic cell, acquiring apoptotic cell-derived antigen (e.g., via nibbling) and activation of proliferation (i.e., crosspriming) of CD8⁺ and CD4⁺ T cells.

“Immune system” is a set of mechanisms that protect an organism from infection by identifying and killing pathogens. It encompasses innate immune system and adaptive immune system. “Innate immune system” provides immune defenses that are non-specific, meaning these systems recognize and respond to pathogens in a generic way. The innate immune system is found in all classes of plant and animal life. It is comprised of the cells and mechanisms that defend the host from infection by other organisms, in a non-specific manner. This means that the cells of the innate system recognize, and respond to, pathogens in a generic way, but unlike the adaptive immune system, it does not confer long-lasting or protective immunity to the host. The inflammatory response is considered part of innate immunity.

“Adaptive immunity” is the ability of vertebrates to recognize pathogens specifically and to provide enhanced protection against reinfection due to immunological memory based on clonal selection of lymphocytes bearing antigen-specific receptors. A process of random recombination of variable receptor gene segments and the pairing of different variable chains generates a population of lymphocytes, each bearing a distinct receptor, forming a repertoire of receptors that can recognize virtually any antigen. If the receptor on a lymphocyte is specific for a ubiquitous self antigen, the cell is normally eliminated by encountering the antigen early in its development. Adaptive immunity is normally initiated when an innate immune response fails to eliminate a new infection, and antigen and activated antigen-presenting cells are delivered to draining lymphoid tissues. When a recirculating lymphocyte encounters its specific foreign antigen in peripheral lymphoid tissues, it is induced to proliferate and its progeny then differentiate into effector cells that can eliminate the infectious agent. A subset of these proliferating lymphocytes differentiate into memory cells, capable of responding rapidly to the same pathogen if it is encountered again.

“Modulators of the immune system or immune responses” encompass any agents that are useful for modulating (antagonizing or agonizing) immune responses in vivo or in vitro. They can modulate immune responses mediated by the innate immunity system or the adaptive immunity system. Agents that can be employed to stimulate or inhibit innate immune responses are termed “modulators of innate immunity” or “agents useful in modulating innate immune response,” e.g., as adjuvants in vaccination or immunesuppressants for inflammation or transplant rejection. Agents that enhance or suppress an antigen specific adaptive immune response are termed “modulators of adaptive immunity” or “agents useful in modulating an antigen-specific immune response.”

“Inflammation” or “inflammatory response” refers to an innate immune response that occurs when tissues are injured by bacteria, trauma, toxins, heat, or any other cause. The damaged tissue releases compounds including histamine, bradykinin, and serotonin. Inflammation refers to both acute responses (i.e., responses in which the inflammatory processes are active) and chronic responses (i.e., responses marked by slow progression and formation of new connective tissue). Acute and chronic inflammation can be distinguished by the cell types involved. Acute inflammation often involves polymorphonuclear neutrophils; whereas chronic inflammation is normally characterized by a lymphohistiocytic and/or granulomatous response. Inflammation includes reactions of both the specific and non-specific defense systems. A specific defense system reaction is a specific immune system reaction response to an antigen (possibly including an autoantigen). A non-specific defense system reaction is an inflammatory response mediated by leukocytes incapable of immunological memory. Such cells include granulocytes, macrophages, neutrophils and eosinophils. Examples of specific types of inflammation are diffuse inflammation, focal inflammation, croupous inflammation, interstitial inflammation, obliterative inflammation, parenchymatous inflammation, reactive inflammation, specific inflammation, toxic inflammation and traumatic inflammation.

“Antagonist” is used in the broadest sense, and includes any molecule that partially or fully blocks, inhibits, or neutralizes a biological activity of apoptotic cell-responding B220⁻ dendritic cell. In a similar manner, the term “agonist” is used in the broadest sense and includes any molecule that mimics or enhances a biological activity of apoptotic cell-responding B220⁻ dendritic cell. Suitable agonist or antagonist molecules specifically include agonist or antagonist antibodies or antibody fragments, fragments or amino acid sequence variants of polypeptides, antisense oligonucleotides, small organic molecules, and the like. Methods for identifying agonists or antagonists of apoptotic cell-responding B220⁻ dendritic cell can comprise contacting a B220 negative dendritic cell with a candidate agonist or antagonist molecule and measuring a detectable change in one or more biological activities normally associated with the B220 negative dendritic cell.

“Autoantigen” is an endogenous antigen that stimulates the production of autoantibodies, as in an autoimmune reaction.

“Signaling in cells” refers to the interaction of a ligand, such as an endogenous or exogenous ligand with receptors of cells such as apoptotic cell-responding B220⁻ dendritic cell which leads to modulation of cellular activities, e.g., an autoimmune response or allogeneic tissue rejection.

“Test compound” or “candidate compound” refers to a nucleic acid, DNA, RNA, protein, polypeptide, or small chemical entity that is determined to effect an increase or decrease in a gene expression as a result of apoptotic cell-responding B220⁻ dendritic cell. The test compound can be an antisense RNA, ribozyme, polypeptide, or small molecular chemical entity. The term “test compound” can be any small chemical compound, or a biological entity, such as a protein, sugar, nucleic acid or lipid. Typically, test compounds will be small chemical molecules and polypeptides. A “test compound specific for apoptotic cell-responding B220⁻ dendritic cell” is determined to be a modulator of cell signaling via a B220 negative dendritic cell or an apoptotic cell.

“Cell-based assays” include, for example, radioligand or fluorescent ligand binding assays for apoptotic cell-responding B220⁻ dendritic cell, e.g., to plasma membranes, detergent-solubilized plasma membrane proteins, immobilized collagen (Alberdi, J Biol Chem. 274:31605-12, 1999; Meyer et al., 2002); TNFRII/CD120b -affinity column chromatography (Alberdi, J Biol Chem. 274:31605-12, 1999; Aymerich et al., Invest Ophihalmol Vis Sci. 42:3287-93, 2001); ligand blot using a radio- or fluorosceinated-ligand (Aymerich el al., Invest Ophihalmol Vis Sci. 42:3287-93, 2001; Meyer et al., 2002); Size-exclusion ultrafiltration (Alberdi et al., Biochem., 1998; Meyer el al., 2002); or ELISA. Exemplary apoptotic cell-responding B220⁻ dendritic cell binding activity assays of the present invention are: a ligand blot assay (Aymerich et al., Invest Ophthalmol Vis Sci. 42:3287-93, 2001); a TNFRII/CD120b affinity column chromatography assay (Alberdi, J Biol Chem. 274:31605-12, 1999) and a ligand binding assay (Alberdi et al., J Biol Chem. 274:31605-12, 1999). Each incorporated by reference in their entirety. Cell based assays further include assays for measurement of cell death-induced CD4⁺ and CD8⁺ T cell responses as described in exemplary embodiments herein. Cell based assays described in exemplary embodiments herein further include immunization and Listeria monocytogenes challenge assay, measurement of cytokine response, generation of DC subsets and T cell purification, determination of priming and cross priming by different DC subsets, isolation of DC subsets from spleen, and immuno-staining of SIINFEKL/K^{b} complex.

“Apoptotic cell” refers to a cell undergoing the process of programmed cell death. Examples of apoptotic cells that can be employed to practice methods of the present invention include splenocytes that are rendered apoptotic through treatment with γ-irradiation, UV-radiation or treatment with Fas-activating antibody, as detailed in the Examples below.

“Type I interferon” refers to a class of interferons, including but not limited to, interferon-α, interferon-β, interferon-κ and interferon-δ. Type II interferon refers to IFN-γ.

In one embodiment, apoptotic cell-responding B220⁻ dendritic cell can be assayed by either immobilizing the ligand or the receptor. For example, the assay can include immobilizing recpetor fused to a His tag onto Ni-activated NTA resin beads. Ligand can be added in an appropriate buffer and the beads incubated for a period of time at a given temperature. After washes to remove unbound material, the bound protein can be released with, for example, SDS, buffers with a high pH, and the like and analyzed.

“Contacting” refers to mixing a test compound in a soluble form into an assay system, for example, a cell-based assay system, such that an effect upon receptor-mediated signaling can be measured. The cell-based system can include one cell type or more than one cell type. Contacting can also refer to combining two or more agents (e.g., polypeptides or small molecule compounds). Contacting can occur in vitro, e.g., combining a test compound with a polypeptide, combining a test compound and a cell or a cell lysate, or combining two different cells. Contacting can also occur in a cell or in situ, e.g., contacting two polypeptides in a cell by coexpression in the cell of recombinant polynucleotides encoding the two polypeptides, or in a cell lysate.

“Detecting an effect” refers to an effect measured in a cell-based assay system. For example, the effect detected can be apoptotic cell-responding B220⁻ dendritic cell in an assay system, for example, a cellular assay, ligand receptor binding assay.

“Assay being indicative of modulation” refers to results of a cell-based assay system indicating that cell activation by apoptotic cell-responding B220⁻ dendritic cell induces a protective response in cells against inflammation.

“Biological activity” and “biologically active” with regard to a ligand of apoptotic cell-responding B220⁻ dendritic cell of the present invention refer to the ability of the ligand molecule to specifically bind to and signal through a native or recombinant receptor, or to block the ability of a native or recombinant receptor to participate in signal transduction. Thus, the (native and variant) ligands of the receptor of the present invention include agonists and antagonists of a native or recombinant receptor. Preferred biological activities of the agonists or antagonists of apoptotic cell-responding B220⁻ dendritic cell of the present invention include the ability to induce or inhibit, for example, inhibiting or enhancing an immune response, or treating autoimmune disease, neoplastic disease, systemic lupus erythematosus, or allogeneic tissue rejection. Accordingly, the administration of the compounds or agents of the present invention can prevent or delay, to alleviate, or to arrest or inhibit development of the symptoms or conditions associated with autoimmune disease, neoplastic disease, allogeneic tissue rejection, or other disorders.

“High affinity” for a ligand refers to an equilibrium association constant (Ka) of at least about 10³M⁻¹, at least about 10⁴M⁻¹, at least about 10⁵M⁻¹, at least about 10⁶ M⁻¹, at least about 10⁷ M⁻¹, at least about 10⁸M⁻¹, at least about 10⁹M⁻¹, at least about 10¹⁰M⁻¹, at least about 10¹¹M⁻¹, or at least about 10¹²M⁻¹ or greater, e.g., up to 10¹³M⁻¹ or 10¹⁴M⁻¹ or greater. However, “high affinity” binding can vary for other ligands.

“Isotype” refers to the antibody class that is encoded by heavy chain constant region genes. Heavy chains are classified as gamma, mu, alpha, delta, or epsilon, and define the antibody's isotype as IgG, IgM, IgA, IgD and IgE, respectively. Additional structural variations characterize distinct subtypes of IgG (e.g., IgG₁, IgG₂, IgG₃ and IgG₄) and IgA (e.g., IgA₁ and IgA₂)

“Kₐ”, as used herein, is intended to refer to the equilibrium association constant of a particular ligand-receptor interaction, e.g., antibody-antigen interaction. This constant has units of 1/M.

“K_{d}”, as used herein, is intended to refer to the equilibrium dissociation constant of a particular ligand-receptor interaction. This constant has units of M.

“kₐ”, as used herein, is intended to refer to the kinetic association constant of a particular ligand-receptor interaction. This constant has units of 1/Ms.

“k_{d}”, as used herein, is intended to refer to the kinetic dissociation constant of a particular ligand-receptor interaction. This constant has units of 1/s.

“Particular ligand-receptor interactions” refers to the experimental conditions under which the equilibrium and kinetic constants are measured.

The ability of a molecule to bind as an agonist or antagonist of apoptotic cell-responding B220⁻ dendritic cell can be determined, for example, by the ability of the putative ligand to bind to a receptor immunoadhesin coated on an assay plate. Specificity of binding can be determined by comparing binding to a B220 negative dendritic cell or an apoptotic cell.

“Control sequences” or “regulatory sequences” refers to DNA sequences necessary for the expression of an operably linked coding sequence in a particular host organism. The control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancers.

“Vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins for which antisera or monoclonal antibodies are available (e.g., the polypeptides of the invention can be made detectable, e.g., by incorporating a radiolabel into the peptide, and used to detect antibodies specifically reactive with the peptide).

“Sorting” in the context of cells as used herein to refers to both physical sorting of the cells, as can be accomplished using, e.g., a fluorescence activated cell sorter, as well as to analysis of cells based on expression of cell surface markers, e.g., FACS analysis in the absence of sorting.

“Cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny cannot be precisely identical in DNA content, due to deliberate or inadvertent mutations. Mutant progeny that have the same function or biological activity as screened for in the originally transformed cell are included. Where distinct designations are intended, it will be clear from the context.

Many types of T cells can be employed to study the ability of apoptotic cell-responding B220⁻ dendritic cell to induce T cell activation. As demonstrated in the Examples below, examples include ovalbumin (OVA) peptide-specific, naïve T cell receptor (TCR) transgenic CD8⁺ T cells (OT-I) (Hogquist et al., Cell 76:17-27, 1994) and CD4⁺ T cells (OT-II) (Barnden et al., Cell Biol. 76:34-40, 1998). Immature CD8⁺ T cells or CD4⁺ T cells which do not express any specific exogenous antigen can also be used in the methods of the invention.

“Antibody” is used in the broadest sense and specifically covers monoclonal antibodies, antibody compositions with polyepitopic specificity, bispecific antibodies, diabodies, and single-chain molecules, as well as antibody fragments (e.g., Fab, F(ab′)₂, and Fv), so long as they exhibit the desired biological activity. Antibodies can be labeled/conjugated to toxic or non-toxic moieties. Toxic moieties include, for example, bacterial toxins, viral toxins, radioisotopes, and the like. Antibodies can be labeled for use in biological assays (e.g., radioisotope labels, fluorescent labels) to aid in detection of the antibody. Antibodies can also be labeled/conjugated for diagnostic or therapeutic purposes, e.g., with radioactive isotopes that deliver radiation directly to a desired site for applications such as radioimmunotherapy (Garmestani. et al., Nucl Med Biol, 28:409, 2001), imaging techniques and radioimmunoguided surgery or labels that allow for in vivo imaging or detection of specific antibody/antigen complexes. Antibodies can also be conjugated with toxins to provide an immunotoxin (see, Kreitman, R J Adv Drug Del Rev, 31:53, 1998).

“Monoclonal antibody” refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention can be made by the hybridoma method first described by Kohler et al., Nature, 256: 495, 1975, or can be made by recombinant DNA methods (see, e.g., U.S. Pat. No. 4,816,567, Cabilly et al.). The “monoclonal antibodies” can also be isolated from phage antibody libraries using the techniques described in Clackson et al., 624-628, 1991, and Marks et al., J. Mol. Biol. 222:581-597, 199 1, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Cabilly et al., supra; Morrison et al., Proc. Natl. Acad. Sci. USA, 81:6851-6855, 1984.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature 321:522-525, 1986; Reichmann et al., Nature 332:323-329, 1988; and Presta, Curr. Op. Struct. Biol. 2:593-596, 1992. The humanized antibody includes a Primatized™ antibody wherein the antigen-binding region of the antibody is derived from an antibody produced by immunizing macaque monkeys with the antigen of interest.

Amino acids from the variable regions of the mature heavy and light chains of immunoglobulins are designated Hx and Lx respectively, where x is a number designating the position of an amino acids according to the scheme of Kabat et al., 1987 and 1991, Sequences of Proteins of Immunological Interest (National Institutes of Health, Bethesda, Md.). Kabat et al. list many amino acid sequences for antibodies for each subclass, and list the most commonly occurring amino acid for each residue position in that subclass. Kabat et al. use a method for assigning a residue number to each amino acid in a listed sequence, and this method for assigning residue numbers has become standard in the field. Kabat et al.'s scheme is extendible to other antibodies not included in the compendium by aligning the antibody in question with one of the consensus sequences in Kabat et al. The use of the Kabat et al. numbering system readily identifies amino acids at equivalent positions in different antibodies. For example, an amino acid at the L50 position of a human antibody occupies the equivalence position to an amino acid position L50 of a mouse antibody

“Non-immunogenic in a human” means that upon contacting the polypeptide of interest in a physiologically acceptable carrier and in a therapeutically effective amount with the appropriate tissue of a human, no state of sensitivity or resistance to the polypeptide of interest is demonstrable upon the second administration of the polypeptide of interest after an appropriate latent period (e.g., 8 to 14 days).

“Neutralizing antibody” refers to an antibody which is able to block or significantly reduce an effector function of wild type or mutant receptor protein or ligand protein. For example, a neutralizing antibody can inhibit or reduce receptor activation by an agonist antibody, as determined, for example, in a receptor/ligand binding assay, or other assays taught herein or known in the art.

“Receptor” denotes a cell-associated protein that binds to a bioactive molecule termed a “ligand.” This interaction mediates the effect of the ligand on the cell. Receptors can be membrane bound, cytosolic or nuclear; monomeric (e.g., thyroid stimulating hormone receptor, beta-adrenergic receptor) or multimeric (e.g., TNF receptor I, TNF receptor II, PDGF receptor, growth hormone receptor, IL-3 receptor, GM-CSF receptor, G-CSF receptor, erythropoietin receptor and IL-6 receptor). Membrane-bound receptors are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor.

In general, the binding of ligand to receptor results in a conformational change in the receptor that causes an interaction between the effector domain and other molecule(s) in the cell, which in turn leads to an alteration in the metabolism of the cell. Metabolic events that are often linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids.

“Treating” or “treatment” refers to any indicia of success in the treatment or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination. Accordingly, the term “treating” includes the administration of the compounds or agents of the present invention to inhibit or enhance an immune response, or treat autoimmune disease, neoplastic disease, systemic lupus erythematosus, or allogeneic tissue rejection. It also includes the administration of the compounds of the present invention to enhance an immune response in a subject toward infection with a pathogen. Accordingly, the term “treating” includes the administration of the compounds or agents of the present invention to prevent or delay, to alleviate, or to arrest or inhibit development of the symptoms or conditions associated autoimmune disease, neoplastic disease, systemic lupus erythematosus, or allogeneic tissue rejection, or other disorders. The term “therapeutic effect” refers to the reduction, elimination, or prevention of the disease, symptoms of the disease, or side effects of the disease in the subject.

“Concomitant administration” of a known drug with a compound of the present invention means administration of the drug and the compound at such time that both the known drug and the compound will have a therapeutic effect or diagnostic effect. Such concomitant administration can involve concurrent (i.e. at the same time), prior, or subsequent administration of the drug with respect to the administration of a compound of the present invention. A person of ordinary skill in the art, would have no difficulty determining the appropriate timing, sequence and dosages of administration for particular drugs and compounds of the present invention.

“Subject” or “patient” refers to any mammalian patient or subject to which the compositions of the invention can be administered. The term mammals, human patients and non-human primates, as well as experimental animals such as rabbits, rats, and mice, and other animals. In an exemplary embodiment, of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that can be associated with the targeted or suspected disease or condition. These and other routine methods allow the clinician to select patients in need of therapy using the methods and formulations of the invention.

By “solid phase” is meant a non-aqueous matrix to which a reagent of interest (e.g., a B220 negative dendritic cell or an apoptotic cell, or an antibody thereto) can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g.,an affinity chromatography column). This term also includes a discontinuous solid phase of discrete particles, such as those described in U.S. Pat. No. 4,275,149.

“Specifically (or selectively) binds” to an antibody refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample.

“Specifically bind(s)” or “bind(s) specifically” when referring to a peptide refers to a peptide molecule which has intermediate or high binding affinity, exclusively or predominately, to a target molecule. The phrase “specifically binds to” refers to a binding reaction which is determinative of the presence of a target protein in the presence of a heterogeneous population of proteins and other biologics. Thus, under designated assay conditions, the specified binding moieties bind preferentially to a particular target protein and do not bind in a significant amount to other components present in a test sample. Specific binding to a target protein under such conditions can require a binding moiety that is selected for its specificity for a particular target antigen. A variety of assay formats can be used to select ligands that are specifically reactive with a particular protein. For example, solid-phase ELISA immunoassays, immunoprecipitation, Biacore and Western blot are used to identify peptides that specifically react with proteins associated with apoptotic cell-responding B220⁻ dendritic cell. Typically a specific or selective reaction will be at least twice background signal or noise and more typically more than 10 times background. Specific binding between a monovalent peptide and a B220 negative dendritic cell or an apoptotic cell means a binding affinity of at least 10³M⁻¹, and preferably 10⁵, 10⁶, 10⁷, 10⁸, 10⁹ or 10¹⁰ M⁻¹. In one embodiment, the binding affinity of is between about 10⁶ M⁻¹ to about 10¹⁰ M⁻¹.

An aspect of the present invention is provided based on the discovery that the B220 negative dendritic cell or the apoptotic cell is a specific sensor of endogenous and exogenous ligands which are necessary for signaling via apoptotic cell-responding B220⁻ dendritic cell. Regulation of signaling via apoptotic cell-responding B220⁻ dendritic cell results in regulation of production of type I interferon in blood cells or tissues of the mammalian subject, or regulation of a cytotoxic T cell response, e.g., a CD8⁺ cytotoxic T cell response in the mammalian subject. Agonists or antagonists that regulate signaling via apoptotic cell-responding B220⁻ dendritic cell are provided which inhibit or enhance an immune response, and therefore are useful for treatment of disease, including, but not limited to, autoimmune disease, neoplastic disease, systemic lupus erythematosus, or allogeneic tissue rejection.

This invention relies on routine techniques in the field of recombinant genetics. Basic texts disclosing the general methods of use in this invention include Sambrook et al., Molecular Cloning, A Laboratory Manual, 2nd ed., 1989; Kriegler, Gene Transfer and Expression. A Laboratory Manual, 1990; and Ausubel et al., eds., , Current Protocols in Molecular Biology, 1994.

Agonist or antagonists of apoptotic cell-responding B220⁻ dendritic cell can be nucleic acids, polymorphic variants, orthologs, and alleles that are substantially identical to sequences provided herein and can be isolated using nucleic acid probes and oligonucleotides under stringent hybridization conditions, by screening libraries. Alternatively, expression libraries can be used to clone agonist or antagonists which are protein, polymorphic variants, orthologs, and alleles by detecting expressed homologs immunologically with antisera or purified antibodies made against apoptotic cell-responding B220⁻ dendritic cell.

Identification of Compounds for Treatment and Prophylaxis of Diseases

As detailed in the Examples below, the present inventors identified that the apoptotic cell-responding B220⁻ dendritic cells are able to activate adaptive immune responses that are independent of Toll-interleukin I receptor signaling pathway. The invention accordingly provides methods for identifying novel agents which can be used to modulate immune responses in a subject. Some of the methods employ cellular assays in conjunction with high throughput screening techniques to screen for antagonists or agonists of the apoptotic cell-responding B220⁻ dendritic cells from candidate agents. General methods for performing such high-throughput screening are provided in the art, e.g., Handbook of Drug Screening, Seethala et al. (eds.), Marcel Dekker (1st ed., 2001); and High Throughput Screening: Methods and Protocols (Methods in Molecular Biology, 190), Janzen (ed.), Humana Press (1 PstP ed., 2002). More specific procedures for carrying such screening methods using the apoptotic cell-responding B220⁻ dendritic cells are disclosed herein.

“Candidate bioactive agent” or “drug candidate” or grammatical equivalents (such as “test agent” or “candidate compound”) as used herein describes any molecule, e.g., protein, oligopeptide, small organic molecule, polysaccharide, polynucleotide, to be tested for bioactive agents that are capable of directly or indirectly altering the activity of apoptotic cell-responding B220⁻ dendritic cell. These terms can encompass a natural product, a synthetic compound, or a chemical compound, or a combination of two or more substances. Unless otherwise specified, the terms “agent”, “substance”, and “compound” can be used interchangeably. In one method, the bioactive agents modulate apoptotic cell-responding B220⁻ dendritic cell. In a further embodiment of the method, the candidate agents induce an antagonist or agonist effect in a receptor ligand assay, as further described below. Generally a plurality of assay mixtures are run in parallel with different agent concentrations to obtain a differential response to the various concentrations. Typically, one of these concentrations serves as a negative control, i.e., at zero concentration or below the level of detection.

Candidate agents encompass numerous chemical classes, though typically they are organic molecules, e.g., small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents can be subjected to directed or random chemical modifications, such as acylation, alkylation, esterification, amidification to produce structural analogs.

In some embodiments, the candidate bioactive agents are proteins. By “protein” herein is meant at least two covalently attached amino acids, which includes proteins, polypeptides, oligopeptides and peptides. The protein can be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the methods herein. “Amino acid” also includes imino acid residues such as proline and hydroxyproline. The side chains can be in either the (R) or the (S) configuration. In further embodiments, the amino acids are in the (S) or (L)-configuration. If non-naturally occurring side chains are used, non-amino acid substituents can be used, for example to prevent or retard in vivo degradations.

In one method, the candidate bioactive agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and mammalian proteins, and human proteins.

In some methods, the candidate bioactive agents are peptides of from about 5 to about 30 amino acids, typically from about 5 to about 20 amino acids, and typically from about 7 to about 15 being. The peptides can be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they can incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In some methods, the library can be fully randomized, with no sequence preferences or constants at any position. In other methods, the library can be biased. Some positions within the sequence are either held constant, or are selected from a limited number of possibilities. For example, in some methods, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, or to purines. In other methods, the candidate bioactive agents are nucleic acids, as defined above.

As described above generally for proteins, nucleic acid candidate bioactive agents can be naturally occurring nucleic acids, random nucleic acids, or “biased” random nucleic acids. For example, digests of procaryotic or eucaryotic genomes can be used as is outlined above for proteins.

In some methods, the candidate bioactive agents are organic chemical moieties.

(A) Drug Screening Methods

The invention provides methods to identify drugs or bioactive agents (i.e., modulators of the immune system) that can modulate the immune system (e.g., innate immunity or adaptive immunity) through modulating biological activities or signaling activities of the apoptotic cell-responding dendritic cells described herein. Some of the methods of the invention entail screening of candidate agents that can act as an antagonist or agonist of apoptotic cell-responding B220⁻ dendritic cell, thus generating the associated phenotype. Candidate agents that can act as an antagonist of apoptotic cell-responding B220⁻ dendritic cell, as shown herein, are expected to result in the immunosuppressive phenotype. Similarly, candidate agents that can act as an agonist of apoptotic cell-responding B220⁻ dendritic cell, as shown herein, are expected to result in the immunostimulant phenotype (e.g., upon challenge with a pathogen).

In one aspect, the invention provides methods of employing the apoptotic cell-responding B220⁻ dendritic cells described herein to screen for compounds that modulate immune responses (innate immune responses or antigen specific immune responses). Some of the methods are directed to identifying compounds that are capable of stimulating or enhancing immune responses (e.g., activation of cytotoxic T cells) mediated by the apoptotic cell-responding B220⁻ dendritic cells. These compounds can be used to stimulate protective responses against pathogens that may elude the immune system. Some of the methods are directed to identifying compounds that are capable of inhibiting or suppressing immune responses (e.g., activation of cytotoxic T cells) mediated by the apoptotic cell-responding B220⁻ dendritic cells. Such modulating compounds are useful to suppress unwanted-responses resulting from, e.g., autoimmune disorders, allergy, inflammation and transplant rejection.

Typically, the screening methods employ the apoptotic cell-responding B220⁻ dendritic cells described herein (e.g., cells isolated from bone marrow or spleen) and screen for compounds which can modulate the signaling activities of the apoptotic cell-responding B220⁻ dendritic cells when stimulated by apoptotic cells. Some of the screening methods are used to identify agents which can be used to modulate (e.g., stimulate) innate immune response which provides immediate defense against infection by other organisms in a non-specific manner. In these methods, no specific antigen is presented by the apoptotic cells in their stimulation of the dendritic cells. As a result, immune responses (e.g., CD8⁺ T cell activation) activated or mediated by the dendritic cells are not antigen specific. Compounds identified in these methods can be used, e.g., in vaccination to modulate (e.g., stimulate) innate immune responses against infections (see, e.g., Werling et al., Vet Immunol Immunopathol. 112:1, 2006).

Some other screening methods of the invention are directed to identifying modulators of antigen-specific adaptive immune responses. In these methods, the apoptotic cells present a specific antigen to stimulate the dendritic cells by, e.g., endogenous expression or internalization of an exogenous antigen. Compounds identified in these methods are used to modulate antigen-specific immune responses against this antigen. For example, the apoptotic cell (e.g., splenocytes rendered apoptotic, e.g., by UV treatment or other methods described herein) can present an antigen of a pathogen such as a bacterium or a virus. These screening methods allow identification of modulating compounds which, when administered to a subject, can enhance the signaling activity of the apoptotic cell-responding B220⁻ dendritic cells and thereby stimulate an antigen specific immune response (e.g., CD8⁺ T cell activation) against the pathogen. In other methods, the apoptotic cell can present an autoantigen, an antigen from a graft tissue, or an antigen that is involved in autoimmune diseases or transplantation rejection (e.g., an HLA antigen). These screening methods are directed to identifying compounds which can ameliorate or prevent graft rejection or autoimmune responses in a subject which have autoimmune disorders or are receiving tissue transplantation (e.g., allogenic graft).

In some exemplary embodiments, the screening methods of the invention comprise first contacting a test compound with an apoptotic cell-responding B220⁻ dendritic cell (e.g., in vitro cultured cell) stimulated with an apoptotic cell, and then detecting a change of a signaling activity of the apoptotic cell-responding B220⁻ dendritic cell relative to its signaling activity in the absence of the test compound (i.e., baseline level activity). A change of signaling activity refers to a significant departure from the baseline level activity, e.g., an increase or decrease by at least 20%, 30%, 40%, 50%, 75%, 100%, 200% or more. If the compound causes a change of a signaling activity of the dendritic cells, it is identified as a potential modulator of innate or adaptive immunity as its effect on the dendritic cells will likely result in modulation of the dendritic cell mediated T cell activation. In some methods, the screening can additionally involve examining effect of the identified compound on innate or adaptive immune responses in vivo or in vitro (e.g., testing whether the compound can modulate CD8⁺ T cell proliferation).

Materials and techniques that are needed for practicing the methods of the invention are all well known in the art or described herein. For example, as detailed in the Examples below, the apoptotic cell-responding B220⁻ dendritic cells can be obtained from FIt3L-induced bone marrow in mice. They can also be CD11b⁻/CD11c⁺ B220⁻ dendritic cell isolated from mouse spleen. Various apoptotic cells can be employed in the practice of the present invention to stimulate the apoptotic cell-responding B220⁻ dendritic cells. These include cells undergoing natural apoptosis and cells rendered apoptotic by biological or physiochemical intervention. Cells can become apoptotic when they are damaged beyond repair, infected with a virus, or undergoing stress conditions such as starvation. Thus, some methods of the invention employ cells rendered apoptotic due to chemical, biological or radioactive treatments in accordance with teachings in the art. For example, treatment with topoisomerase I inhibitor camptothecin induces apoptosis of human promyelocytic leukemia HL60 cells (Shimizu et al., Cancer Res. 55:228-31, 1995). Certain spore-extracted toxins can induce apoptosis of a mouse alveolar macrophage cell line MH-S (see, e.g., Wang and Yaday, Toxicol Appl Pharmacol. 214:297-308, 2006). Oxidative stress caused by prostaglandin can induce apoptosis of a thyroid papillary cancer cell line (CG3 cells) (see, e.g., Chen et al., Anticancer Drugs 13:759-65, 2002).

Cells undergoing apoptosis due to viral infection can also be utilized in the methods of the present invention. For example, human monocytic cell line (THP-1) can become apoptotic when infected by measles virus (see, e.g., Ito et al., FEMS Immunol Med Microbiol. 15(2-3): 115-22, 1996). Human T lymphocyte virus I infection can lead to apoptosis of human lymphocytes (Leno et al., J Exp Med. 181:1575-80, 1995). CHSE-214, a Chinook salmon embryonic cell line, undergoes apoptosis when infected by infectious pancreatic necrosis virus (see, e.g., Hong et al., Virology 250:76-84, 1998). In addition, tumor cells can be rendered apoptotic by treatments with chemical or biological agents. For example, antitumor agent cisplatin can induce apoptosis of many types of human tumor cells. Examples include human neuroblastoma cell line (see, e.g., Cece et al., Anticancer Res. 15:777-82, 1995), human ovarian carcinoma cell line (see, e.g., Ormerod et al., Exp Cell Res. 211:231-7, 1994), and human melanoma cell line (Zhao et al., Anticancer Drugs 6:657-68, 1995). Other agents such as paclitaxel have been shown to induce apoptosis in several human gastric carcinoma cell lines (see, e.g., Chang et al., Cancer 77:14-18, 1996). Any of these established cell lines which undergo apoptosis may be used in the screening methods of the invention.

In some preferred embodiments, splenocytes obtained from a mammalian subject (e.g., mouse) and rendered apoptotic are employed to stimulate the apoptotic cell-responding B220⁻ dendritic cells. As demonstrated herein, the splenocytes can become apoptotic by treatment such as γ-irradiation, UV-irradiation or Fas-activating antibody. Detailed procedures for isolating splenocytes and inducing apoptotic properties in the cells are disclosed in the Examples below (e.g., Example 8). Induction of apoptosis in these cells can be examined by, e.g., flow cytometry using apoptotic markers such as annexin V and propidium iodide, as described below.

As noted above, some methods of the invention employ apoptotic cells (e.g., irradiated splenocytes) which comprise a specific antigen, e.g., antigens that are involved in graft rejection or pathogenic infections. For example, hepatitis B surface antigen plays an important role in HBV viral infection of host cells. This antigen can be employed to screen for compounds which modulate (e.g., stimulate) immune responses against HBV infection. Similarly, HIV-1 antigen gp120 can be used to screen for compounds that stimulate antigen specific immune responses against HIV infection. Other pathenogic antigens that can be used include bacterial antigens, e.g., cholera protective antigen (CPA) and anthracis protective antigen. Some other methods of the invention employ antigens that are involved in transplantation rejections. Examples of such antigens include the DFFRY gene encoded antigen which is involved in human bone marrow graft rejection (see, e.g., Vogt et al., Blood. 95:1100-5, 2000). Similarly, the UTY gene encodes a human male-specific minor histocompatibility antigen that is involved in stem cell graft rejection (Vogt et al., Blood. 96:3126-32, 2000). Many other examples are known in the art.

Apoptotic cells that present or comprise (e.g., via endogenous expression) an exogenous antigen can be prepared using methods routinely practiced in the art. For example, are exemplified for the act-OVA antigen in the Examples, splenocytes expressing a specific antigen can be prepared by transgenic technology (e.g., using transgenic mice) (see, e.g., Ehst et al., Am. J. Transplant. 3:1355-62, 2003) and then rendered apoptotic by UV treatment or other treatments described herein. Apoptotic cells comprising the antigen can also be prepared via recombinant expression of the antigen in the cells, using standard recombinant technology well known in the art. Further, splenocytes presenting an antigen against which immune response is to be modulated by the compounds to be identified can also be prepared by culturing the cells in the presence of the antigen. The antigen can be internalized by the splenocytes by endocytosis or phagocytosis. The cells can then be rendered apoptotic by, e.g., radioactive treatment. Techniques for enabling cultured cells to internalize external antigens are well known in the art (see, e.g., Selby et al., 1: Cell Immunol. 1995 163:47-54, 1995).

In some screening methods of the invention, test compounds are screened for activity in stimulating or increasing (i.e., up-regulate) the signaling activities of the apoptotic cell-responding B220⁻ dendritic cells. In some other methods, test compounds are examined for ability to suppress or inhibit (i.e., down-regulate) the signaling activities of the apoptotic cell-responding B220⁻ dendritic cells. In some methods, the test compounds can be incubated or contacted with the apoptotic cell-responding B220⁻ dendritic cells prior to stimulation with the apoptotic cell. In some methods, the dendritic cells are contacted with the test compounds simultaneously with or subsequent to stimulation with the apoptotic cell.

The screening methods of the present invention can monitor test compounds' effect on any of the signaling activities of the apoptotic cell-responding B220⁻ dendritic cells in eliciting Toll-interleukin I receptor-independent T cell activation described herein. In some methods, the signaling activity examined is production of type I interferon (e.g., IFN-α or IFN-β) by the dendritic cell upon stimulation with the apoptotic cell. Type I IFN production by the cells can be quantified using methods well known and routinely practiced in the art. As exemplified in the Examples below, one such method is to determine the concentration of type I IFN in the supernatant of the dendritic cells cocultured with the apoptotic cells using assays described in, e.g., Jiang et al., Nat. Immunol. 6:565-570, 2005. In some other methods, signaling activity of the apoptotic cell-responding B220⁻ dendritic cells to be monitored is uptake by the dendritic cell of apoptotic material from the apoptotic cell. For example, the apoptotic cells (e.g., UV-treated splenocytes) employed to stimulate the dendritic cells can be labeled with a fluorescent marker. Uptake by the dendritic cells of antigens from the apoptotic cells can be quantitated by fluorescence-activated cell sorting (FACS), as illustrated in the Examples herein.

In still some other methods, signaling activity of the dendritic cells employed in the screening is activation of T cell proliferation. As described in detail in the Examples below, either CD8⁺ T cell or CD4⁺ T cell responses can be measured to monitor signaling activity of the apoptotic cell-responding B220⁻ dendritic cells. These can be accomplished in vitro by examining activity of the dendritic cells in priming purified OT-I CD8⁺ T-cells or OT-II CD4⁺ T-cells. As exemplified herein (e.g., Example 8), the dendritic cells can be first stimulated with the apoptotic cells prior to contacting with the purified T cells. The T cells can be labeled with a fluorescent marker such as CFSE so that proliferation of the T cells can be quantitated via, e.g., flow-cytometry analysis.

In another aspect, the invention provides screening methods for identifying agents which can alter other biological activities or function of the apoptotic cell-responding B220⁻ dendritic cell. Again, having identified the importance of a B220 negative dendritic cell or an apoptotic cell, screening for agents that bind and/or modulate the biological activity of the apoptotic cell-responding B220⁻ dendritic cell can be performed as outlined below. Some of the methods are aimed at screening for candidate agents that modulate apoptotic cell-responding B220⁻ dendritic cell either at the level of gene expression or protein level.

In some methods, a candidate agent can be administered in any one of several cellular assays, e.g., receptor-ligand binding assay. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e., a peptide) can be put into a viral construct such as a retroviral construct and added to the cell, such that expression of the peptide agent is accomplished; see PCT US97/01019, incorporated herein by reference in its entirety.

Once the candidate agent has been administered to the cells, the cells can be washed if desired and are allowed to incubate under physiological conditions for some period of time. The cells are then harvested and a new gene expression profile is generated, as outlined herein.

For example, assay systems for apoptotic cell-responding B220⁻ dendritic cell can be screened for agents that produce an immunosuppressive or immune-stimulating phenotype. A change in a binding assay or cellular assay indicates that the agent has an effect on apoptotic cell-responding B220⁻ dendritic cell. In one method, an immunosuppressive or immune-stimulating profile is induced or maintained, before, during, and/or after stimulation with ligand. By defining such a signature for inhibiting or enhancing an immune response, or treating autoimmune disease, , systemic lupus erythematosus, allogeneic tissue rejection, infectious disease, or neoplastic disease screens for new drugs that mimic the an immunosuppressive or immune-stimulating phenotype can be devised. With this approach, the drug target need not be known and need not be represented in the original expression screening platform, nor does the level of transcript for the target protein need to change. In some methods, the agent acts as an agonist or antagonist in one of several cellular or binding assays, e.g., binding assay to measure apoptotic cell-responding B220⁻ dendritic cell.

In some methods, screens can be done on individual genes and gene products. After having identified a cellular or binding assay as indicative of inhibition or enhancement of an immune response, or treatment of autoimmune disease, neoplastic disease, systemic lupus erythematosus, or allogeneic tissue rejection, screening of modulators of cellular or binding assay can be completed.

Thus, in some methods, screening for modulators of cellular or binding assay can be completed. This will be done as outlined above, but in general a few cellular or binding assay are evaluated. In some methods, screens are designed to first find candidate agents that can affect a cellular activity or binding assay, and then these agents can be used in other assays that evaluate the ability of the candidate agent to modulate apoptotic cell-responding B220⁻ dendritic cell.

In general, purified or isolated gene product can be used for binding assays; that is, the gene products of receptors or ligands associated with apoptotic cell-responding B220⁻ dendritic cell. Using the nucleic acids of the methods and compositions herein which encode receptor protein or ligand protein, a variety of expression vectors can be made. The expression vectors can be either self-replicating extrachromosomal vectors or vectors which integrate into a host genome. Generally, these expression vectors include transcriptional and translational regulatory nucleic acid operably linked to the nucleic acid encoding a receptors or ligands associated with apoptotic cell-responding B220⁻ dendritic cell. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express receptors or ligands protein associated with apoptotic cell-responding B220⁻ dendritic cell; for example, transcriptional and translational regulatory nucleic acid sequences from Bacillus are used to express the protein in Bacillus or other bacterial or eukaryotic expression system. Numerous types of appropriate expression vectors, and suitable regulatory sequences are known in the art for a variety of host cells.

In general, the transcriptional and translational regulatory sequences can include, but are not limited to, promoter sequences, ribosomal binding sites, transcriptional start and stop sequences, translational start and stop sequences, and enhancer or activator sequences. In one method, the regulatory sequences include a promoter and transcriptional start and stop sequences.

Promoter sequences encode either constitutive or inducible promoters. The promoters can be either naturally occurring promoters or hybrid promoters. Hybrid promoters, which combine elements of more than one promoter, are also known in the art, and are useful in the methods herein.

In addition, the expression vector can comprise additional elements. For example, the expression vector can have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and typically two homologous sequences which flank the expression construct. The integrating vector can be directed to a specific locus in the host cell by selecting the appropriate homologous sequence for inclusion in the vector. Constructs for integrating vectors are well known in the art. Methods to effect homologous recombination are described in PCT US93/03868 and PCT US98/05223, each incorporated herein by reference in their entirety.

In some methods, the expression vector contains a selectable marker gene to allow the selection of transformed host cells. Selection genes are well known in the art and will vary with the host cell used.

One expression vector system is a retroviral vector system such as is generally described in PCT/US97/01019 and PCT/US97/01048, each incorporated herein by reference in their entirety.

The receptor proteins or ligand proteins of apoptotic cell-responding B220⁻ dendritic cell of the present methods and compositions are produced by culturing a host cell transformed with an expression vector containing nucleic acid encoding receptor proteins or ligand proteins, under the appropriate conditions to induce or cause expression of the protein. The conditions appropriate for receptor protein or ligand protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In some methods, the timing of the harvest is important. For example, the baculoviral systems used in insect cell expression are lytic viruses, and thus harvest time selection can be crucial for product yield.

Appropriate host cells include yeast, bacteria, archebacteria, fungi, and insect and animal cells, including mammalian cells. Of particular interest are Drosophila melanogaster cells, Saccharomyces cerevisiae and other yeasts, E. coli, Bacillus subtilis, SF9 cells, C129 cells, 293 cells, Neurospora, BHK, CHO, COS, and HeLa cells. In some methods, CD4+CD25+^{hi} T_{R} cells are host cells as provided herein, which for example, include non-recombinant cell lines, such as primary cell lines. In addition, purified B220 negative dendritic cells or apoptotic cells for receptor ligand assays derived from either transgenic or non-transgenic strains can also be used. The host cell can alternatively be an cell type known to have immunodeficiency disorder.

In one method, the receptor proteins or ligand proteins are expressed in mammalian cells. Mammalian expression systems can include retroviral systems. A mammalian promoter is any DNA sequence capable of binding mammalian RNA polymerase and initiating the downstream (3′) transcription of a coding sequence for receptor proteins or ligand proteins into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenlytion signals include those derived form SV40.

The methods of introducing nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In some methods, receptor proteins or ligand proteins are expressed in bacterial systems which are well known in the art.

In other methods, receptor proteins or ligand proteins can be produced in insect cells. Expression vectors for the transformation of insect cells, and in particular, baculovirus-based expression vectors, are well known in the art.

In some methods, receptor proteins or ligand proteins are produced in yeast cells. Yeast expression systems are well known in the art, and include expression vectors for Saccharomyces cerevisiae, Candida albicans and C. maltosa, Hansenula polymorpha, Kluyveromycesfragilis and K. lactis, Pichia guillerimondii and P. pastoris, Schizosaccharomyces pombe, and Yarrowia lipolytica.

A receptor protein or a ligand protein can also be made as a fusion protein, using techniques well known in the art. For example, for the creation of monoclonal antibodies, if the desired epitope is small, the protein can be fused to a carrier protein to form an immunogen. Alternatively, receptor proteins or ligand proteins can be made as a fusion protein to increase expression. For example, when a protein is a shorter peptide, the nucleic acid encoding the peptide can be linked to other nucleic acid for expression purposes. Similarly, receptor proteins or ligand proteins of the methods and compositions herein can be linked to protein labels, such as green fluorescent protein (GFP), red fluorescent protein (RFP), yellow fluorescent protein (YFP), and blue fluorescent protein (BFP).

In one embodiment, the proteins are recombinant. A “recombinant protein” is a protein made using recombinant techniques, i.e., through the expression of a recombinant nucleic acid as depicted above. A recombinant protein is distinguished from naturally occurring protein by at least one or more characteristics. For example, the protein can be isolated or purified away from some or all of the proteins and compounds with which it is normally associated in its wild type host, and thus can be substantially pure. For example, an isolated protein is unaccompanied by at least some of the material with which it is normally associated in its natural state, typically constituting at least about 0.5%, typically at least about 5% by weight of the total protein in a given sample. A substantially pure protein comprises at least about 75% by weight of the total protein, at least about 80%, and typically at least about 90%. The definition includes the production of receptor proteins or ligand proteins from one organism in a different organism or host cell. Alternatively, the protein can be made at a significantly higher concentration than is normally seen, through the use of a inducible promoter or high expression promoter, such that the protein is made at increased concentration levels. Alternatively, the protein can be in a form not normally found in nature, as in the addition of an epitope tag or amino acid substitutions, insertions and deletions, as discussed below.

In some methods, when the receptor proteins or ligand proteins are to be used to generate antibodies, the protein must share at least one epitope or determinant with the full length transcription product of the nucleic acids. By “epitope” or “determinant” herein is meant a portion of a protein which will bind an antibody. Thus, in most instances, antibodies made to a smaller protein should be able to bind to the full length protein. In one embodiment, the epitope is unique; that is, antibodies generated to a unique epitope show little or no cross-reactivity.

In some methods, the antibodies provided herein can be capable of reducing or eliminating the biological function of receptor proteins or ligand proteins in apoptotic cell-responding B220⁻ dendritic cell, as is described below. The addition of antibodies (either polyclonal or monoclonal) to the protein (or cells containing the protein) can reduce or eliminate the protein's activity. Generally, at least a 25% decrease in activity is observed, with typically at least about 50% and typically about a 95-100% decrease being observed.

In addition, the proteins can be variant proteins, comprising one more amino acid substitutions, insertions and deletions.

In one method, receptor proteins or ligand proteins are purified or isolated after expression. Proteins can be isolated or purified in a variety of ways. Standard purification methods include electrophoretic, molecular, immunological and chromatographic techniques, including ion exchange, hydrophobic, affinity, and reverse-phase HPLC chromatography, and chromatofocusing. For example, receptor proteins or ligand proteins can be purified using a standard antibody affinity column. Ultrafiltration and diafiltration techniques, in conjunction with protein concentration, are also useful. For general guidance in suitable purification techniques, see Scopes, Protein Purification, Springer-Verlag, NY, 1982, incorporated herein by reference in its entirety. The degree of purification necessary will vary depending on the use of the protein. In some instances no purification will be necessary.

Once the gene product of the receptor protein or ligand protein gene is made, binding assays can be done. These methods comprise combining a receptor protein or a ligand protein and a candidate bioactive agent, and determining the binding of the candidate agent to the receptor protein or ligand protein. Methods utilize a human receptor protein or ligand protein to interact in apoptotic cell-responding B220⁻ dendritic cell, although other mammalian proteins can also be used, including rodents (mice, rats, hamsters, guinea pigs), farm animals (cows, sheep, pigs, horses) and primates. These latter methods can be used for the development of animal models of human disease. In some methods, variant or derivative receptor proteins or ligand proteins can be used, including deletion receptor proteins or ligand proteins as outlined above.

The assays herein utilize receptor proteins or ligand proteins as defined herein. In some assays, portions of proteins can be utilized. In other assays, portions having different activities can be used. In addition, the assays described herein can utilize either isolated receptor proteins or ligand proteins or cells comprising the receptor proteins or ligand proteins. In some methods, the protein or the candidate agent is non-diffusably bound to an insoluble support having isolated sample receiving areas (e.g., a microtiter plate or an array). The insoluble supports can be made of any composition to which the compositions can be bound, is readily separated from soluble material, and is otherwise compatible with the overall method of screening. The surface of such supports can be solid or porous and of any convenient shape. Examples of suitable insoluble supports include microtiter plates, arrays, membranes and beads. These are typically made of glass, plastic (e.g., polystyrene), polysaccharides, nylon or nitrocellulose, and teflon™. Microtiter plates and arrays are especially convenient because a large number of assays can be carried out simultaneously, using small amounts of reagents and samples. In some cases magnetic beads and the like are included. The particular manner of binding of the composition is not crucial so long as it is compatible with the reagents and overall methods described herein, maintains the activity of the composition and is nondiffusable. Methods of binding include the use of antibodies (which do not sterically block either the ligand binding site or activation sequence when the protein is bound to the support), direct binding to ionic supports, chemical crosslinking, or by the synthesis of the protein or agent on the surface. Following binding of the protein or agent, excess unbound material is removed by washing. The sample receiving areas can then be blocked through incubation with bovine serum albumin (BSA), casein or other innocuous protein or other moiety. Also included in the methods and compositions herein are screening assays wherein solid supports are not used.

In other methods, the receptor proteins or ligand proteins are bound to the support, and a candidate bioactive agent is added to the assay. Alternatively, the candidate agent is bound to the support and the protein is added. Novel binding agents include specific antibodies, non-natural binding agents identified in screens of chemical libraries, and peptide analogs. Of particular interest are screening assays for agents that have a low toxicity for human cells. A wide variety of assays can be used for this purpose, including labeled in vitro protein-protein binding assays, electrophoretic mobility shift assays, immunoassays for protein binding, functional assays (such as phosphorylation assays) and the like.

The determination of the binding of the candidate bioactive agent to receptor proteins or ligand proteins determining apoptotic cell-responding B220⁻ dendritic cell can be done in a number of ways. In some methods, the candidate bioactive agent is labeled, and binding determined directly. For example, this can be done by attaching all or a portion of a receptor protein or a ligand protein to a solid support, adding a labeled candidate agent (for example a fluorescent label), washing off excess reagent, and determining whether the label is present on the solid support. Various blocking and washing steps can be utilized.

By “labeled” herein is meant that the compound is either directly or indirectly labeled with a label which provides a detectable signal, e.g., radioisotope, fluorescers, enzyme, antibodies, particles such as magnetic particles, chemiluminescers, or specific binding molecules. Specific binding molecules include pairs, such as biotin and streptavidin, digoxin and antidigoxin. For the specific binding members, the complementary member would normally be labeled with a molecule which provides for detection, in accordance with known procedures, as outlined above. The label can directly or indirectly provide a detectable signal.

In some methods, only one of the components is labeled. For example, the proteins (or proteinaceous candidate agents) can be labeled at tyrosine positions using ¹²⁵I, or with fluorophores. Alternatively, more than one component can be labeled with different labels; using ¹²⁵I for the proteins, for example, and a fluorophor for the candidate agents.

In other methods, the binding of the candidate bioactive agent is determined through the use of competitive binding assays. In this method, the competitor is a binding moiety known to bind to the target molecule such as an antibody, peptide, binding partner, or ligand. Under certain circumstances, there can be competitive binding as between the bioactive agent and the binding moiety, with the binding moiety displacing the bioactive agent. This assay can be used to determine candidate agents which interfere with binding between proteins and the competitor.

In some methods, the candidate bioactive agent is labeled. Either the candidate bioactive agent, or the competitor, or both, is added first to the protein for a time sufficient to allow binding, if present. Incubations can be performed at any temperature which facilitates optimal activity, typically between about 4° C. and 40° C. Incubation periods are selected for optimum activity, but can also be optimized to facilitate rapid high through put screening. Typically between 0.1 and 1 hour will be sufficient. Excess reagent is generally removed or washed away. The second component is then added, and the presence or absence of the labeled component is followed, to indicate binding.

In other methods, the competitor is added first, followed by the candidate bioactive agent. Displacement of the competitor is an indication that the candidate bioactive agent is binding to the receptor protein or ligand protein and thus is capable of binding to, and potentially modulating, the activity of the protein. In this method, either component can be labeled. For example, if the competitor is labeled, the presence of label in the wash solution indicates displacement by the agent. Alternatively, if the candidate bioactive agent is labeled, the presence of the label on the support indicates displacement.

In other methods, the candidate bioactive agent is added first, with incubation and washing, followed by the competitor. The absence of binding by the competitor can indicate that the bioactive agent is bound to receptor proteins or ligand proteins with a higher affinity. Thus, if the candidate bioactive agent is labeled, the presence of the label on the support, coupled with a lack of competitor binding, can indicate that the candidate agent is capable of binding to the protein.

Competitive binding methods can also be run as differential screens. These methods can comprise receptor proteins or ligand proteins and a competitor in a first sample. A second sample comprises a candidate bioactive agent, a receptor protein or a ligand protein and a competitor. The binding of the competitor is determined for both samples, and a change, or difference in binding between the two samples indicates the presence of an agent capable of binding to the receptor proteins or ligand proteins and potentially modulating its activity. If the binding of the competitor is different in the second sample relative to the first sample, the agent is capable of binding to the protein.

Other methods utilize differential screening to identify drug candidates that bind to the native receptor proteins or ligand proteins, but cannot bind to modified proteins. The structure of the protein can be modeled, and used in rational drug design to synthesize agents that interact with that site. Drug candidates that affect apoptotic cell-responding B220⁻ dendritic cell are also identified by screening drugs for the ability to either enhance or reduce the activity of the protein.

In some methods, screening for agents that modulate the activity of proteins or cells are performed. In general, this will be done on the basis of the known biological activity of the receptor proteins or ligand proteins involved in apoptotic cell-responding B220⁻ dendritic cells or other signaling activities of the cells. In these methods, a candidate bioactive agent is added to a sample of the protein or an assay system comprising the cells, as above, and an alteration in the biological activity of the protein is determined. “Modulation” or “modulating the activity” includes an increase in activity, a decrease in activity, or a change in the type or kind of activity present. Thus, in these methods, the candidate agent should both bind receptor proteins or ligand proteins (although this may not be necessary), and alter its biological or biochemical activity as defined herein. The methods include both in vitro screening methods, as are generally outlined above, and in vivo screening of cells for alterations in the presence, distribution, activity or amount of the protein.

Some methods comprise combining a receptor proteins or ligand proteins involved in cell signaling via a B220 negative dendritic cell or an apoptotic cell and a candidate bioactive agent, then evaluating the effect on cell signaling via apoptotic cell-responding B220⁻ dendritic cell to inhibit or enhance an immune response. By “cell signaling via a B220 negative dendritic cell or an apoptotic cell” or grammatical equivalents herein is meant one of a B220 negative dendritic cell or an apoptotic cell biological activities, including, but not limited to, its ability to affect immune activation or inhibition. One activity herein is the capability to bind to a target gene, or modulate apoptotic cell-responding B220⁻ dendritic cell. Cell signaling via apoptotic cell-responding B220⁻ dendritic cell is induced or maintained.

In other methods, the activity of the receptor proteins or ligand proteins are increased; in other methods, the activity of the receptor proteins or ligand proteins are decreased. Thus, bioactive agents that are antagonists are useful in some methods, and bioactive agents that are agonists are useful in other methods.

Methods for screening for bioactive agents capable of modulating the activity of receptor proteins or ligand proteins are provided. These methods comprise adding a candidate bioactive agent, as defined above, to a cell comprising proteins. Cell types include almost any cell. The cells contain a recombinant nucleic acid that encodes receptor proteins or ligand proteins involved in apoptotic cell-responding B220⁻ dendritic cell. In one method, a library of candidate agents are tested on a plurality of cells. The effect of the candidate agent on signaling activity via apoptotic cell-responding B220⁻ dendritic cell is then evaluated.

Positive controls and negative controls can be used in the assays. All control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples can be counted in a scintillation counter to determine the amount of bound compound.

A variety of other reagents can be included in the screening assays. These include reagents like salts, neutral proteins (e.g., albumin and detergents) which can be used to facilitate optimal protein-protein binding and/or reduce non-specific or background interactions. Reagents that otherwise improve the efficiency of the assay, (such as protease inhibitors, nuclease inhibitors, anti-microbial agents) can also be used. The mixture of components can be added in any order that provides for the requisite binding.

The components provided herein for the assays provided herein can also be combined to form kits. The kits can be based on the use of the protein and/or the nucleic acid encoding the receptor proteins or ligand proteins. Assays regarding the use of nucleic acids are further described below.

(B) Animal Models

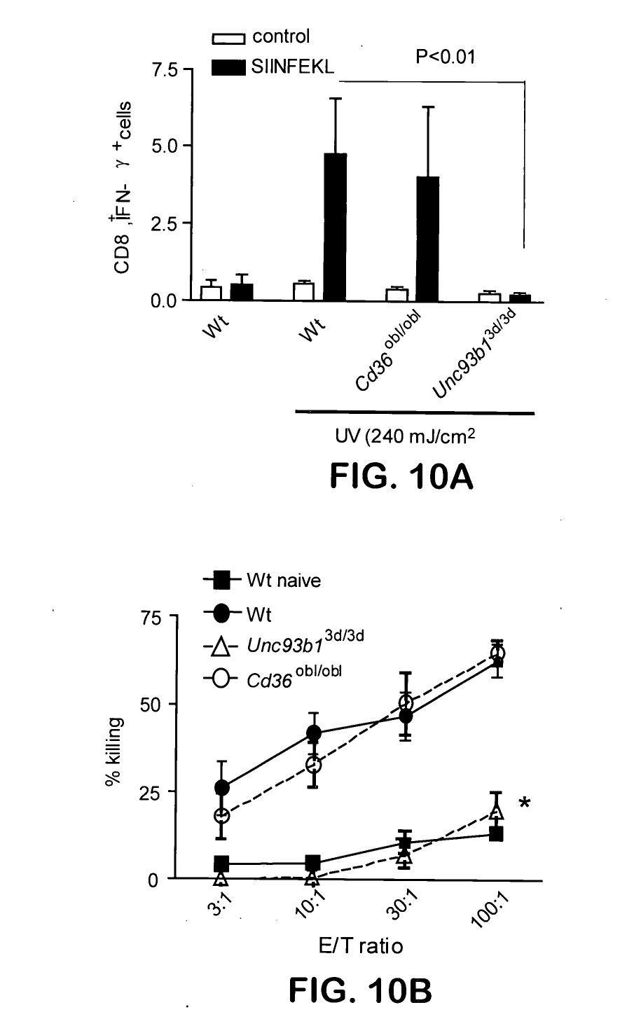

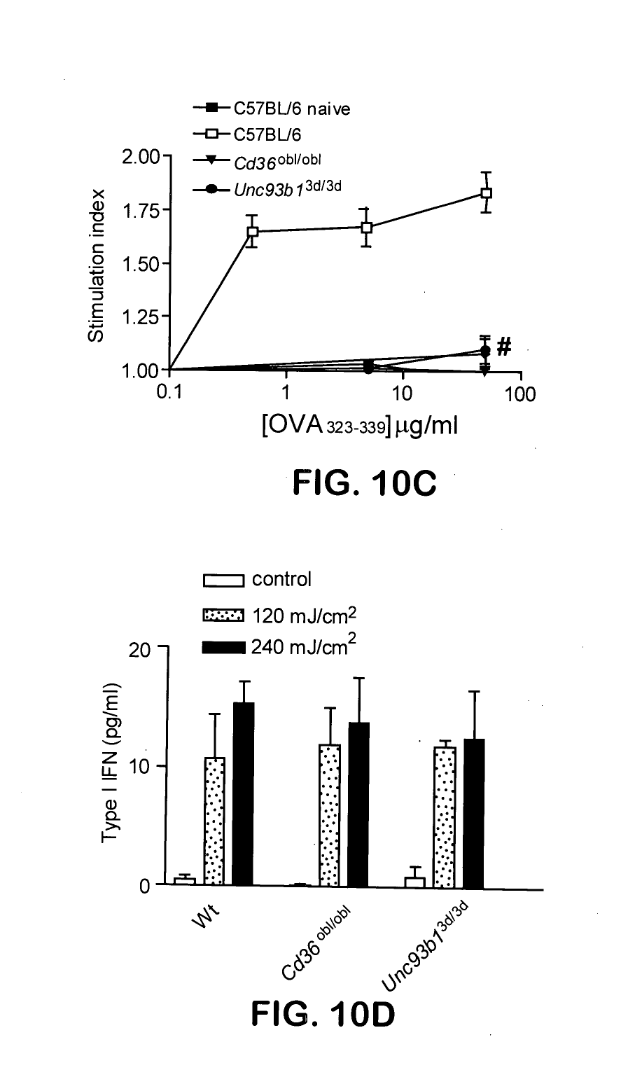

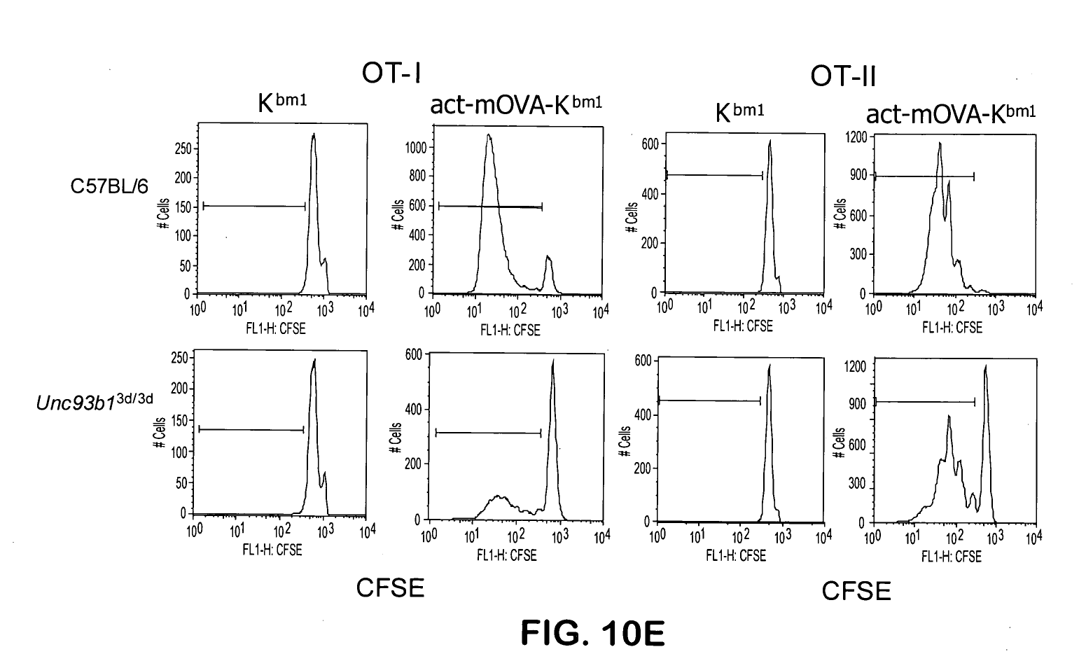

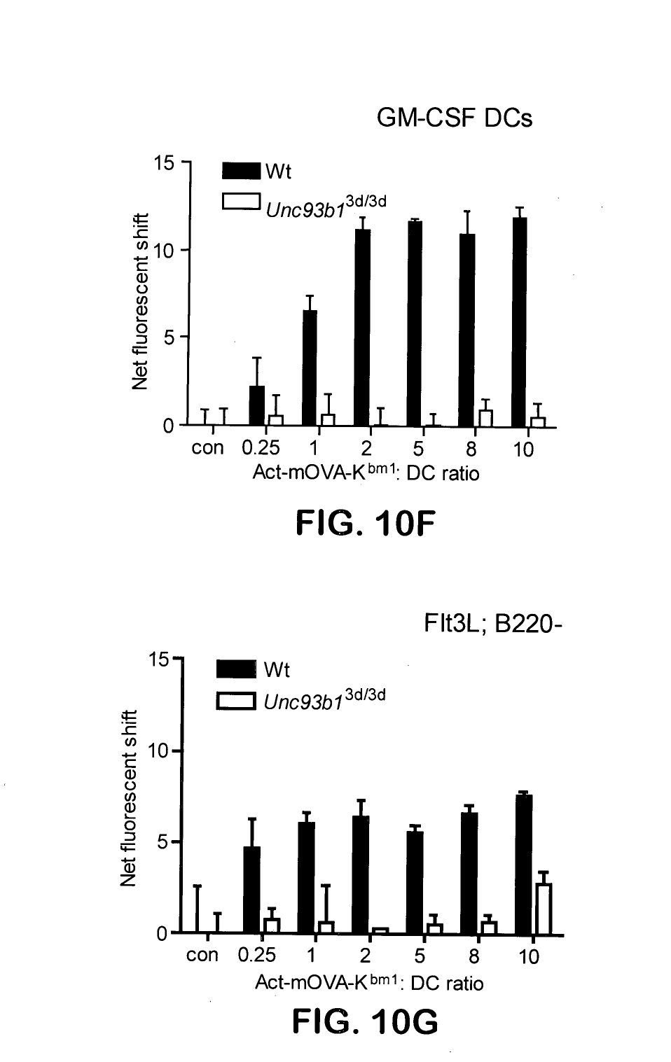

Germline mutagenesis of C57BL/6 mice using N-ethyl-N-nitrosourea (ENU) was utilized to isolate mutant mice that are deficient in apoptotic cell-induced immune responses. Hoebe et al., J Endotoxin Res 9:250-5, 2003; Hoebe, K. et al., Nature 424: 743-748, 2003. Among numerous ENU-induced mutations known to affect innate immune responses, two exhibited significant inhibitory effects on adaptive responses induced by apoptotic cells. As discussed in more detail in the exemplary embodiments, mutatins in CD36 and UNC-93B play a role in apoptotic cell-induced immune responses. Oblivious a nonsense allele of Cd36 known to impair sensing of microbial diacylglycerides, abrogated CD4 responses, but not CD8 responses, in homozygous mutant mice. Hoebe et al., Nature 433: 523-527, 2005. Strikingly, the mutation had no effect on type I IFN production. The 3d allele of Unc93bl, which encodes UNC-93B, a 12-spanning ER membrane protein required for responses to TLR3, 7 and 9 ligands, abrogated both CD8⁺ and CD4⁺ responses in homozygotes. Tabeta et al., Nat. Immunol. in press, 2006. Again, type I IFN production was unaffected. See FIG. 10.

In one method, nucleic acids which encode receptor proteins or ligand proteins involved in apoptotic cell-responding B220⁻ dendritic cell or their modified forms can also be used to generate either transgenic animals, including “knock-in” and “knock out” animals which, in turn, are useful in the development and screening of therapeutically useful reagents. A non-human transgenic animal (e.g., a mouse or rat) is an animal having cells that contain a transgene, which transgene is introduced into the animal or an ancestor of the animal at a prenatal, e.g., an embryonic stage. A transgene is a DNA which is integrated into the genome of a cell from which a transgenic animal develops, and can include both the addition of all or part of a gene or the deletion of all or part of a gene. In some methods, cDNA encoding receptor proteins or ligand proteins can be used to clone genomic DNA encoding receptor proteins or ligand proteins in accordance with established techniques and the genomic sequences used to generate transgenic animals that contain cells which either express (or overexpress) or suppress the desired DNA. Methods for generating transgenic animals, particularly animals such as mice or rats, have become conventional in the art and are described, for example, in U.S. Pat. Nos. 4,736,866 and 4,870,009, each incorporated herein by reference in their entirety. Typically, particular cells would be targeted for a receptor protein or a ligand protein transgene incorporation with tissue-specific enhancers. Transgenic animals that include a copy of a transgene encoding a receptor protein or a ligand protein introduced into the germ line of the animal at an embryonic stage can be used to examine the effect of increased expression of the desired nucleic acid. Such animals can be used as tester animals for reagents thought to confer protection from, for example, pathological conditions associated with its overexpression. In accordance with this facet, an animal is treated with the reagent and a reduced incidence of the pathological condition, compared to untreated animals bearing the transgene, would indicate a potential therapeutic intervention for the pathological condition. Similarly, non-human homologues of receptor proteins or ligand proteins can be used to construct a transgenic animal comprising a protein “knock out” animal which has a defective or altered gene encoding a receptor protein or a ligand protein as a result of homologous recombination between the endogenous gene encoding a receptor protein or a ligand protein and altered genomic DNA encoding the protein introduced into an embryonic cell of the animal. For example, cDNA encoding receptor proteins or ligand proteins can be used to clone genomic DNA encoding the protein in accordance with established techniques. A portion of the genomic DNA encoding receptor proteins or ligand proteins can be deleted or replaced with another gene, such as a gene encoding a selectable marker which can be used to monitor integration. Typically, several kilobases of unaltered flanking DNA (both at the 5′ and 3′ ends) are included in the vector (see, e.g., Thomas and Capecchi, Cell 51:503, 1987, incorporated herein by reference in its entirety, for a description of homologous recombination vectors). The vector is introduced into an embryonic stem cell line (e.g., by electroporation) and cells in which the introduced DNA has homologously recombined with the endogenous DNA are selected (see, e.g., Li el al., Cell 69:915, 1992, incorporated herein by reference in its entirety). The selected cells are then injected into a blastocyst of an animal (e.g., a mouse or rat) to form aggregation chimeras (see, e.g., Bradley, in Teratocarcinomas and Embryonic Stem Cells: A Practical Approach, E. J. Robertson, ed. (IRL, Oxford, 1987), pp. 113-152). A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of receptor proteins or ligand proteins involved in cell signaling via a B220 negative dendritic cell or an apoptotic cell polypeptide.

Animal models for cell signaling related disorders, or having a particular state of cell signaling activity via a B220 negative dendritic cell or an apoptotic cell can include, for example, genetic models. For example, such animal models for autoimmune diseases can include the nonobese diabetic (NOD) mouse (see, e.g., McDuffie, Curr Opin Immunol. 10(6):704-9, 1998; Tochino, Crit Rev Immunol 8(1):49-81, 1987), and experimental autoimmune encephalomyelitis (EAE) (see, e.g., Wong, Immunol Rev 169:93-104, 1999). See also Schwartz et al., Autoimmunity and Autoimmune Diseases, Ch. 31, in Fundamental Immunology, Paul (ed.), Raven Press 1989, each incorporated herein by reference in their entirety. Other models can include studies involving transplant rejection.

The role signaling in B220 negative dendritic cell recognition of an apoptotic cell relating to treatment of infectious disease was demonstrated in an in vivo murine model Mice were immunized and challenged with Listeria monocytogenes. L. monocytogenes titer was determined in the spleen of infected and immunized mice.

Animal models exhibiting a cell signaling related disorder-like symptoms can be engineered by utilizing, for example, receptor protein or ligand protein sequences in conjunction with techniques for producing transgenic animals that are well known to those of skill in the art. For example, gene sequences can be introduced into, and overexpressed in, the genome of the animal of interest, or, if endogenous target gene sequences are present, they can either be overexpressed or, alternatively, can be disrupted in order to underexpress or inactivate target gene expression.

In order to overexpress a target gene sequence, the coding portion of the target gene sequence can be ligated to a regulatory sequence which is capable of driving gene expression in the animal and cell type of interest. Such regulatory regions will be well known to those of skill in the art, and can be utilized in the absence of undue experimentation.

For underexpression of an endogenous target gene sequence, such a sequence can be isolated and engineered such that when reintroduced into the genome of the animal of interest, the endogenous target gene alleles will be inactivated. The engineered target gene sequence is introduced via gene targeting such that the endogenous target sequence is disrupted upon integration of the engineered target sequence into the animal's genome.

Animals of any species, including, but not limited to, mice, rats, rabbits, guinea pigs, pigs, micro-pigs, goats, and non-human primates, e.g., baboons, monkeys, and chimpanzees can be used to generate animal models of cell signaling related disorders or being a perpetually desired state of the cell signaling via a B220 negative dendritic cell or an apoptotic cell.

(C) Nucleic Acid Based Therapeutics

Nucleic acids encoding receptor polypeptides or ligand polypeptides, antagonists or agonists can also be used in gene therapy. Broadly speaking, a gene therapy vector is an exogenous polynucleotide which produces a medically useful phenotypic effect upon the mammalian cell(s) into which it is transferred. A vector can or can not have an origin of replication. For example, it is useful to include an origin of replication in a vector for propagation of the vector prior to administration to a patient. However, the origin of replication can often be removed before administration if the vector is designed to integrate into host chromosomal DNA or bind to host mRNA or DNA. Vectors used in gene therapy can be viral or nonviral. Viral vectors are usually introduced into a patient as components of a virus. Nonviral vectors, typically dsDNA, can be transferred as naked DNA or associated with a transfer-enhancing vehicle, such as a receptor-recognition protein, lipoamine, or cationic lipid.

Viral vectors, such as retroviruses, adenoviruses, adenoassociated viruses and herpes viruses, are often made up of two components, a modified viral genome and a coat structure surrounding it (see generally Smith et al., Ann. Rev. Microbiol. 49:807-838, 1995, incorporated herein by reference in its entirety), although sometimes viral vectors are introduced in naked form or coated with proteins other than viral proteins. Most current vectors have coat structures similar to a wildtype virus. This structure packages and protects the viral nucleic acid and provides the means to bind and enter target cells. However, the viral nucleic acid in a vector designed for gene therapy is changed in many ways. The goals of these changes are to disable growth of the virus in target cells while maintaining its ability to grow in vector form in available packaging or helper cells, to provide space within the viral genome for insertion of exogenous DNA sequences, and to incorporate new sequences that encode and enable appropriate expression of the gene of interest. Thus, vector nucleic acids generally comprise two components: essential cis-acting viral sequences for replication and packaging in a helper line and the transcription unit for the exogenous gene. Other viral functions are expressed in trans in a specific packaging or helper cell line.

Nonviral nucleic acid vectors used in gene therapy include plasmids, RNAs, antisense oligonucleotides (e.g., methylphosphonate or phosphorothiolate), polyamide nucleic acids, interfering RNA (RNAi), hairpin RNA, and yeast artificial chromosomes (YACs). Such vectors typically include an expression cassette for expressing a protein or RNA. The promoter in such an expression cassette can be constitutive, cell type-specific, stage-specific, and/or modulatable (e.g., by hormones such as glucocorticoids; MMTV promoter). Transcription can be increased by inserting an enhancer sequence into the vector. Enhancers are cis-acting sequences of between 10 to 300 bp that increase transcription by a promoter. Enhancers can effectively increase transcription when either 5′ or 3′ to the transcription unit. They are also effective if located within an intron or within the coding sequence itself. Typically, viral enhancers are used, including SV40 enhancers, cytomegalovirus enhancers, polyoma enhancers, and adenovirus enhancers. Enhancer sequences from mammalian systems are also commonly used, such as the mouse immunoglobulin heavy chain enhancer.

Gene therapy vectors can be delivered in vivo by administration to an individual patient, typically by systemic administration (e.g., intravenous, intraperitoneal, intramuscular, subdermal, or intracranial infusion) or topical application. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Modulating Signaling in Apoptotic Cell-Responding B220⁻ Dendritic Cell

(A) Assays for Modulators of Signaling in Apoptotic Cell-Responding B220⁻ Dendritic Cell

In numerous embodiments of this invention, the level of signaling in apoptotic cell-responding B220⁻ dendritic cell will be modulated in a cell by administering to the cell, in vivo or in vitro, any of a large number of agonist or antagonist molecules, e.g., polypeptides, antibodies, amino acids, nucleotides, lipids, carbohydrates, or any organic or inorganic molecule.

To identify molecules capable of modulating signaling in apoptotic cell-responding B220⁻ dendritic cell, assays will be performed to detect the effect of various compounds on receptor ligand signaling activity in a cell. Receptor ligand signaling can be assessed using a variety of in vitro and in vivo assays to determine functional, chemical, and physical effects, e.g., measuring the binding of receptor or ligand to other molecules (e.g., radioactive binding), measuring protein and/or RNA levels of signaling pathway in apoptotic cell-responding B220⁻ dendritic cell that provides an immunosuppressive or immune-stimulating response, or measuring other aspects of pathway signaling, e.g., phosphorylation levels, transcription levels, receptor activity, ligand binding and the like. Such assays can be used to test for both activators and inhibitors of signaling. Modulators thus identified are useful for, e.g., many diagnostic and therapeutic applications.

The signaling pathway in apoptotic cell-responding B220⁻ dendritic cell in the assay will typically be a recombinant or naturally occurring polypeptide or a conservatively modified variant thereof. Alternatively, the signaling pathway in apoptotic cell-responding B220⁻ dendritic cell in the assay will be derived from a eukaryote and include an amino acid subsequence having amino acid sequence identity to the naturally occurring pathway signaling. Generally, the amino acid sequence identity will be at least 70%, optionally at least 75%, 85%, or 86%, 87%, 88%, 89%, 90%, 91%, 92%, 93%, 94%, 95%, 96%, 97%, 98%, or greater. Optionally, the polypeptide of the assays will comprise a domain of an receptor or ligand of the pathway. In certain embodiments, a domain of the receptor or ligand is bound to a solid substrate and used, e.g., to isolate any molecules that can bind to and/or modulate their activity. In certain embodiments, a domain of a receptor or ligand polypeptide, e.g., an N-terminal domain, a C-terminal domain, is fused to a heterologous polypeptide, thereby forming a chimeric polypeptide. Such chimeric polypeptides are also useful, e.g., in assays to identify modulators of pathway signaling in apoptotic cell-responding B220⁻ dendritic cell.

The terms “identical” or percent “identity,” in the context of two or more nucleic acids or polypeptide sequences, refers to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequence encoding a receptor or ligand described herein or amino acid sequence of a receptor or ligand described herein), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site). Such sequences are then said to be “substantially identical.” This term also refers to, or can be applied to, the compliment of a test sequence. The term also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482, 1981 by the homology alignment algorithm of Needleman & Wunsch, J. Mol. Biol. 48:443, 1970 by the search for similarity method of Pearson & Lipman, Proc. Nat'l. Acad. Sci. 85:2444, 1988 by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology, eds. 1995 supplement)).

A preferred example of algorithm that is suitable for determining percent sequence identity and sequence similarity are the BLAST and BLAST 2.0 algorithms, which are described in Altschul et al., Nuc. Acids Res 25:3389-3402, 1977 and Altschul et al., J. Mol. Biol. 215:403-410, 1990 respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nim.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. 89:10915, 1989) alignments (B) of 50, expectation (E) of 10, M=5, N=−4, and a comparison of both strands.

The terms “polypeptide”, “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (1), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part 1: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity, e.g., a kinase domain. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript can be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1 993). Generally, stringent conditions are selected to be about 5-10° C. lower than the thermal melting point (Tₘ) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions can also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., Ausubel el al, supra.

For PCR, a temperature of about 36° C. is typical for low stringency amplification, although annealing temperatures can vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. PCR Protocols, A Guide to Methods and Applications, Academic Press, Inc. N.Y. (1990).

Samples or assays that are treated with a potential inhibitor or activator of signaling in apoptotic cell-responding B220⁻ dendritic cell are compared to control samples without the test compound, to examine the extent of modulation. Control samples (untreated with activators or inhibitors) are assigned a relative signaling activity value of 100. Inhibition of signaling in apoptotic cell-responding B220⁻ dendritic cell is achieved when the pathway signaling activity value relative to the control is about 90%, optionally about 50%, optionally about 25-0%. Activation of signaling in apoptotic cell-responding B220⁻ dendritic cell is achieved when the pathway signaling activity value relative to the control is about 110%, optionally about 150%, 200-500%, or about 1000-2000%.

The effects of the test compounds upon the function of the polypeptides can be measured by examining any of the parameters described above. Any suitable physiological change that affects signaling activity in apoptotic cell-responding B220⁻ dendritic cell can be used to assess the influence of a test compound on the polypeptides of this invention. When the functional consequences are determined using intact cells or animals, one can also measure a variety of effects such as changes in cell growth or changes in cell-cell interactions.

Modulators of signaling in apoptotic cell-responding B220⁻ dendritic cell that act by modulating gene expression can also be identified. For example, a host cell containing a B220 negative dendritic cell protein of interest or apoptotic cell protein of interest is contacted with a test compound for a sufficient time to effect any interactions, and then the level of gene expression is measured. The amount of time to effect such interactions can be empirically determined, such as by running a time course and measuring the level of transcription as a function of time. The amount of transcription can be measured using any method known to those of skill in the art to be suitable. For example, mRNA expression of the protein of interest can be detected using Northern blots or by detecting their polypeptide products using immunoassays.

(B) Assays for Compounds that Affect Signaling in Apoptotic Cell-Responding B220⁻ Dendritic Cell

In certain embodiments, assays will be performed to identify molecules that physically interact with receptor or ligand for signaling in apoptotic cell-responding B220⁻ dendritic cell. Such molecules can be any type of molecule, including polypeptides, polynucleotides, amino acids, nucleotides, carbohydrates, lipids, or any other organic or inorganic molecule. Such molecules can represent molecules that normally interact with a receptor or a ligand or can be synthetic or other molecules that are capable of interacting with a receptor or a ligand and that can potentially be used as lead compounds to identify classes of molecules that can interact with and/or modulate signaling in apoptotic cell-responding B220⁻ dendritic cell. Such assays can represent physical binding assays, such as affinity chromatography, immunoprecipitation, two-hybrid screens, or other binding assays, or can represent genetic assays.

In any of the binding or functional assays described herein, in vivo or in vitro, for detection of signaling in apoptotic cell-responding B220⁻ dendritic cell, or any derivative, variation, homolog, or fragment of a receptor or a ligand can be used. Preferably, the receptor or ligand has at least about 85% identity to the amino acid sequence of the naturally occurring receptor or ligand. In numerous embodiments, a fragment of a receptor or a ligand is used. Such fragments can be used alone, in combination with other receptor or ligand protein fragments, or in combination with sequences from heterologous proteins, e.g., the fragments can be fused to a heterologous polypeptides, thereby forming a chimeric polypeptide.

Compounds that interact to effect signaling in apoptotic cell-responding B220⁻ dendritic cell can be isolated based on an ability to specifically bind to a a receptor or a ligand or fragment thereof. In numerous embodiments, the receptor or ligand or protein fragment will be attached to a solid support. In one embodiment, affinity columns are made using the receptor or ligand polypeptide, and physically-interacting molecules are identified. It will be apparent to one of skill that chromatographic techniques can be performed at any scale and using equipment from many different manufactures (e.g., Pharmacia Biotechnology). In addition, molecules that interact with a receptor or a ligand in vivo can be identified by co-immunoprecipitation or other methods, i.e., immunoprecipitating receptor or ligand using antibodies to receptor or ligand from a cell or cell extract, and identifying compounds, e.g., proteins, that are precipitated along with the receptor or ligand. Such methods are well known to those of skill in the art and are taught, e.g., in Ausubel et al., 1994; Sambrook et al., Molecular Cloning. A Laboratory Manual, Cold Spring Harbor Press, NY., 1989; and Harlow and Lane, Antibodies: A Laboratory Manual, Cold Spring Harbor Press, NY., 1989.

(C) Increasing Receptor or Ligand Protein Activity Levels that Effect Signaling in Apoptotic Cell-Responding B220⁻ Dendritic Cell

In certain embodiments, this invention provides methods of treating infectious disease or neoplastic disease by increasing signaling in apoptotic cell-responding B220⁻ dendritic cell or protein levels affecting signaling in a cell. Typically, such methods are used to increase a reduced level of receptor or ligand protein, e.g., a reduced level in a B220 negative dendritic cell or an apoptotic cell, and can be performed in any of a number of ways, e.g., increasing the copy number of receptor or ligand genes or increasing the level of receptor or ligand mRNA, protein, or protein activity in a cell. Preferably, the level of protein activity is increased to a level typical of a normal, cell, but the level can be increased to any level that is sufficient to increase signaling in apoptotic cell-responding B220⁻ dendritic cell, including to levels above or below those typical of normal cells. Preferably, such methods involve the use of activators of B220 negative dendritic cell or apoptotic cell, where an “activator of B220 negative dendritic cell or apoptotic cell” is a molecule that acts to increase type I interferon gene polynucleotide levels, polypeptide levels and/or protein activity or increases a cytotoxic T cell response as a CD8⁺ T cell response. Such activators can include, but are not limited to, antibody activators or small molecule activators of signaling in apoptotic cell-responding B220⁻ dendritic cell.

(D) Reducing Receptor or Ligand Protein Activity Levels that Effect Signaling in Apoptotic Cell-Responding B220⁻ Dendritic Cell

In certain embodiments, this invention provides methods of treating autoimmune disease or allogeneic tissue rejection by reducing signaling in apoptotic cell-responding B220⁻ dendritic cell or reducing receptor or ligand protein levels in a cell. Typically, such methods are used to reduce an elevated level of a receptor or a ligand protein, e.g., an elevated level in a B220 negative dendritic cell or apoptotic cell can be performed in any of a number of ways, e.g., lowering the copy number of a receptor or a ligand protein genes or decreasing the level of mRNA, protein, or protein activity in a cell. Preferably, the level of receptor or ligand protein activity is lowered to a level typical of a normal B220 negative dendritic cell or apoptotic cell, but the level can be reduced to any level that is sufficient to decrease signaling in apoptotic cell-responding B220⁻ dendritic cell, including to levels above or below those typical of normal cells. Preferably, such methods involve the use of inhibitors of receptor or ligand protein, where an “inhibitor of receptor or ligand” is a molecule that acts to reduce receptor or ligand protein polynucleotide levels, polypeptide levels and/or protein activity. Such inhibitor s include, but are not limited to, antisense polynucleotides, ribozymes, antibodies, dominant negative receptor or ligand protein forms, and small molecule inhibitors of receptor or ligand protein.

In preferred embodiments, receptor or ligand protein levels or signaling in apoptotic cell-responding B220⁻ dendritic cell will be reduced so as to treat autoimmune disease or allogeneic tissue rejection as a result of elevated receptor or ligand protein levels. The proliferation of a cell refers to the rate at which the cell or population of cells divides, or to the extent to which the cell or population of cells divides or increases in number. Proliferation can reflect any of a number of factors, including the rate of cell growth and division and the rate of cell death. Without being bound by the following offered theory, it is suggested that the amplification and/or overexpression of the receptor or ligand induced gene in B220 negative dendritic cells or apoptotic cells to inhibit or enhance an immune response, or treat autoimmune disease or allogeneic tissue rejection signaling. Inhibition or activation of immune activity via altered receptor ligand interaction can act to treat autoimmune disease, allogeneic tissue, or rejection. The ability of any of the present compounds to affect receptor or ligand protein activity can be determined based on any of a number of factors, including, but not limited to, a level of receptor or ligand polynucleotide, e.g., mRNA or gDNA, the level of receptor or ligand polypeptide, the degree of binding of a compound to a receptor or ligand polynucleotide or polypeptide, receptor or ligand protein intracellular localization, or any functional properties of receptor or ligand protein, such as the ability of receptor or ligand protein activity to inhibit or enhance an immune response, or treat autoimmune disease, or allogeneic tissue rejection.

Preferably, such methods involve the use of inhibitors of signaling in apoptotic cell-responding B220⁻ dendritic cell, where an “inhibitor of signaling in apoptotic cell-responding B220⁻ dendritic cell” is a molecule that acts to decrease type I interferon gene polynucleotide levels, polypeptide levels and/or protein activity or decreases a cytotoxic T cell response as a CD8⁺ T cell response. Such inhibitors can include, but are not limited to, antibody inhibitors or small molecule inhibitors of signaling in apoptotic cell-responding B220⁻ dendritic cell.

(E) Inhibitors of Receptor or Ligand Affecting Signaling in Apoptotic Cell-Responding B220⁻ Dendritic Cell

In certain embodiments, receptor or ligand protein activity is downregulated, or entirely inhibited, by the use of antisense polynucleotide, i.e., a nucleic acid complementary to, and which can preferably hybridize specifically to, a coding mRNA nucleic acid sequence, e.g., receptor or ligand induced mRNA, or a subsequence thereof. Binding of the antisense polynucleotide to the mRNA reduces the translation and/or stability of the receptor or ligand induced mRNA.

In the context of this invention, antisense polynucleotides can comprise naturally-occurring nucleotides, or synthetic species formed from naturally-occurring subunits or their close homologs. Antisense polynucleotides can also have altered sugar moieties or inter-sugar linkages. Exemplary among these are the phosphorothioate and other sulfur containing species which are known for use in the art. All such analogs are comprehended by this invention so long as they function effectively to hybridize with receptor or ligand induced mRNA.

Such antisense polynucleotides can readily be synthesized using recombinant means, or can be synthesized in vitro. Equipment for such synthesis is sold by several vendors, including Applied Biosystems. The preparation of other oligonucleotides such as phosphorothioates and alkylated derivatives is also well known to those of skill in the art.

In addition to antisense polynucleotides, ribozymes can be used to target and inhibit transcription of receptor protein or ligand protein that effect signaling in apoptotic cell-responding B220⁻ dendritic cell. A ribozyme is an RNA molecule that catalytically cleaves other RNA molecules. Different kinds of ribozymes have been described, including group I ribozymes, hammerhead ribozymes, hairpin ribozymes, RNAse P, and axhead ribozymes (see, e.g., Castanotto et al., Adv. in Pharmacology 25: 289-317, 1994 for a general review of the properties of different ribozymes).

The general features of hairpin ribozymes are described, e.g., in Hampel et al., Nucl. Acids Res., 18: 299-304, 1990; Hampel et al., European Patent Publication No. 0 360 257, 1990; U.S. Pat. No. 5,254,678. Methods of preparing are well known to those of skill in the art (see, e.g., Wong-Staal et al., WO 94/26877; Ojwang et al., Proc. Natl. Acad. Sci. USA, 90: 6340-6344, 1993; Yamada et al., Human Gene Therapy 1: 39-45, 1994; Leavitt et al., Proc. Natl. Acad. Sci. USA, 92: 699-703, 1995; Leavitt et al., Human Gene Therapy 5: 1151-120, 1994; and Yamada et al., Virology 205: 121-126, 1994).

Receptor or ligand activity can also be decreased by the addition of an inhibitor of the receptor or ligand protein. This can be accomplished in any of a number of ways, including by providing a dominant negative receptor or ligand polypeptide, e.g., a form of receptor or ligand protein that itself has no activity and which, when present in the same cell as a functional receptor or ligand protein, reduces or eliminates the receptor or ligand protein activity of the functional receptor or ligand protein. Design of dominant negative forms is well known to those of skill and is described, e.g., in Herskowitz, Nature 329:219-22, 1987. Also, inactive polypeptide variants (muteins) can be used, e.g., by screening for the ability to inhibit receptor or ligand protein activity. Methods of making muteins are well known to those of skill (see, e.g., U.S. Pat. Nos. 5,486,463; 5,422,260; 5,116,943; 4,752,585; and 4,518,504). In addition, any small molecule, e.g., any peptide, amino acid, nucleotide, lipid, carbohydrate, or any other organic or inorganic molecule can be screened for the ability to bind to or inhibit receptor or ligand protein activity, as described below.

(F) Modulators and Binding Compounds

The compounds tested as modulators of receptor protein or ligand protein that affect signaling in apoptotic cell-responding B220⁻ dendritic cell can be any small chemical compound, or a biological entity, such as a protein, sugar, nucleic acid or lipid. Typically, test compounds will be small chemical molecules and peptides. Essentially any chemical compound can be used as a potential modulator or binding compound in the assays of the invention, although most often compounds can be dissolved in aqueous or organic (especially DMSO-based) solutions. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In one preferred embodiment, high throughput screening methods involve providing a combinatorial chemical or peptide library containing a large number of potential therapeutic compounds (potential modulator or binding compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka, Int. J. Pept. Prot. Res. 37:487-493, 1991; and Houghton et al., Nature 354: 84-88, 1991). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913, 1993), vinylogous polypeptides (Hagihara et al., J. Amer. Chem. Soc. 114:6568, 1992), nonpeptidal peptidomimetics with glucose scaffolding (Hirschmann et al., J Amer. Chem. Soc. 114:9217-9218, 1992), analogous organic syntheses of small compound libraries (Chen et al., J. Amer. Chem. Soc. 116:2661, 1994), oligocarbamates (Cho et al., Science 261:1303, 1993), and/or peptidyl phosphonates (Campbell et al., J. Org. Chem. 59:658, 1994), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology 14:309-314, 1996; and PCT/US96/10287), carbohydrate libraries (see, e.g., Liang et al., Science, 274:1520-1522, 1996; and U.S. Pat. No. 5,593,853), small organic molecule libraries (see, e.g., benzodiazepines, Baum, C&EN, page 33, Jan. 18, 1993; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J.; Tripos, Inc., St. Louis, Mo.; 3D Pharmaceuticals, Exton, Pa.; Martek Biosciences, Columbia, Md., etc.).

(G) Solid State and Soluble High Throughput Assays

In one embodiment, the invention provides soluble assays using molecules such as an N-terminal or C-terminal domain either alone or covalently linked to a heterologous protein to create a chimeric molecule. In another embodiment, the invention provides solid phase based in vitro assays in a high throughput format, where a domain, chimeric molecule, receptor protein or ligand protein, or cell or tissue expressing a receptor or ligand protein is attached to a solid phase substrate.

In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 different compounds is possible using the integrated systems of the invention. More recently, microfluidic approaches to reagent manipulation have been developed.

The molecule of interest can be bound to the solid state component, directly or indirectly, via covalent or non covalent linkage, e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154, 1993 (describing solid phase synthesis of, e.g., peptides); Geysen et al., J. Immun. Meth. 102:259-274, 1987 (describing synthesis of solid phase components on pins); Frank & Doring, Tetrahedron 44:6031-6040, 1988 (describing synthesis of various peptide sequences on cellulose disks); Fodor et al., Science 251: 767-777, 1991; Sheldon et al., Clinical Chemistry 39:718-719, 1993; and Kozal et al., Nature Medicine 2:753-759, 1996 (all describing arrays of biopolymers fixed to solid substrates). Nonchemical approaches for fixing tag binders to substrates include other common methods, such as heat, cross-linking by UV radiation, and the like.

(H) Rational Drug Design Assays

Yet another assay for compounds that modulate receptor or ligand protein activity affecting signaling in apoptotic cell-responding B220⁻ dendritic cell involves computer assisted drug design, in which a computer system is used to generate a three-dimensional structure of a receptor or ligand protein based on the structural information encoded by its amino acid sequence. The input amino acid sequence interacts directly and actively with a pre-established algorithm in a computer program to yield secondary, tertiary, and quaternary structural models of the protein. The models of the protein structure are then examined to identify regions of the structure that have the ability to bind. These regions are then used to identify compounds that bind to the protein.

The three-dimensional structural model of the protein is generated by entering protein amino acid sequences of at least 10 amino acid residues or corresponding nucleic acid sequences encoding a receptor or ligand polypeptide into the computer system. The nucleotide sequence encoding the polypeptide, or the amino acid sequence thereof, and conservatively modified versions thereof, of the naturally occurring gene sequence. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential modulator binding regions are identified by the computer system. Three-dimensional structures for potential modulators are generated by entering amino acid or nucleotide sequences or chemical formulas of compounds, as described above. The three-dimensional structure of the potential modulator is then compared to that of the receptor or ligand protein to identify compounds that bind to the protein. Binding affinity between the protein and compound is determined using energy terms to determine which compounds have an enhanced probability of binding to the protein.

Computer systems are also used to screen for mutations, polymorphic variants, alleles and interspecies homologs of receptor or ligand induced genes. Such mutations can be associated with disease states or genetic traits. GeneChip™ and related technology can also be used to screen for mutations, polymorphic variants, alleles and interspecies homologs. Once the variants are identified, diagnostic assays can be used to identify patients having such mutated genes. Identification of the mutated receptor or ligand induced genes involves receiving input of a first nucleic acid or amino acid sequence of the naturally occurring receptor or ligand induced gene, respectively, and conservatively modified versions thereof. The sequence is entered into the computer system as described above. The first nucleic acid or amino acid sequence is then compared to a second nucleic acid or amino acid sequence that has substantial identity to the first sequence. The second sequence is entered into the computer system in the manner described above. Once the first and second sequences are compared, nucleotide or amino acid differences between the sequences are identified. Such sequences can represent allelic differences in various receptor or ligand induced genes, and mutations associated with disease states and genetic traits.

Diagnostic Methods

In addition to assays, the creation of animal models, and nucleic acid based therapeutics, identification of important genes allows the use of these genes in diagnosis (e.g., diagnosis of cell states and abnormal cell conditions). Disorders based on mutant or variant receptor or ligand genes that affect signaling in apoptotic cell-responding B220⁻ dendritic cell can be determined. Methods for identifying cells containing variant receptor or ligand genes comprising determining all or part of the sequence of at least one endogeneous genes in a cell are provided. As will be appreciated by those in the art, this can be done using any number of sequencing techniques. Methods of identifying the genotype of an individual comprising determining all or part of the sequence of at least one receptor or ligand gene of the individual are also provided. This is generally done in at least one tissue of the individual, and can include the evaluation of a number of tissues or different samples of the same tissue. The method can include comparing the sequence of the sequenced mutant receptor or ligand gene to a known receptor or ligand gene, i.e., a wild-type gene.

The sequence of all or part of the receptor or ligand gene in a patient with disease can then be compared to the sequence of a known receptor or ligand gene to determine if any differences exist. This can be done using any number of known sequence identity programs, such as Bestfit, and others outlined herein. In some methods, the presence of a difference in the sequence between the receptor or ligand gene of the patient and the known receptor or ligand gene is indicative of a disease state or a propensity for a disease state, as outlined herein.

Similarly, diagnosis of B220 negative dendritic cell states or apoptotic cell states can be done using the methods and compositions herein. By evaluating the gene expression profile of B220 negative dendritic cells or apoptotic cells from a patient, the B220 negative dendritic cell state or apoptotic cell state can be determined. This is particularly useful to verify the action of a drug, for example an immunosuppressive drug or a drug to treat autoimmune disease, infectious disease, or neoplastic disease. Other methods comprise administering the drug to a patient and removing a cell sample, particularly of B220 negative dendritic cells or apoptotic cells, from the patient. The gene expression profile of the cell is then evaluated, as outlined herein, for example by comparing it to the expression profile from an equivalent sample from a healthy individual. In this manner, both the efficacy (i.e., whether the correct expression profile is being generated from the drug) and the dose (is the dosage correct to result in the correct expression profile) can be verified.

The present discovery relating to the role of agonists or antagonists of signaling in apoptotic cell-responding B220⁻ dendritic cell in inhibiting or enhancing an immune response, or e.g., treating autoimmune disease, neoplastic disease, infectious disease, or allogeneic tissue rejection thus provides methods for treating differing disease states. In one method, the receptor or ligand proteins, and particularly receptor or ligand protein fragments, are useful in the study or treatment of conditions which are mediated by various disease states, i.e., to diagnose, treat or prevent immune-mediated disorders. Thus, “immune-mediated disorders” or “disease states” can include conditions involving, for example, inhibition or enhancement of an immune response, autoimmune disease, neoplastic disease, infectious disease, or allogeneic tissue rejection.

Methods of modulating immune-regulatory states in cells or organisms are provided. Some methods comprise administering to a cell an anti-receptor or anti-ligand antibody or other agent identified herein or by the methods provided herein, that reduces or eliminates the biological activity of the endogeneous receptor or ligand protein. Alternatively, the methods comprise administering to a cell or organism a recombinant nucleic acid encoding a receptor or ligand protein or modulator including anti-sense nucleic acids. As will be appreciated by those in the art, this can be accomplished in any number of ways. In some methods, the activity of receptor signaling in apoptotic cell-responding B220⁻ dendritic cell is increased by increasing the amount or activity of receptor or ligand in the cell, for example by overexpressing the endogeneous receptor protein or ligand protein or by administering a receptor or ligand gene, using known gene therapy techniques, for example. In one method, the gene therapy techniques include the incorporation of the exogenous gene using enhanced homologous recombination (EHR), for example as described in PCT/US93/03868, hereby incorporated by reference in its entirety.

Methods for diagnosing a B220 negative dendritic cell or apoptotic cell activity related condition in an individual are provided. The methods comprise measuring the activity of receptor or ligand protein in a tissue from the individual or patient, which can include a measurement of the amount or specific activity of the protein. This activity is compared to the activity of receptor or ligand protein from either an unaffected second individual or from an unaffected tissue from the first individual. When these activities are different, the first individual can be at risk for a cell activity mediated disorder involving signaling in B220 negative dendritic cell recognition of apoptotic.

Furthermore, nucleotide sequences encoding a receptor or ligand protein can also be used to construct hybridization probes for mapping the gene which encodes that receptor or ligand protein and for the genetic analysis of individuals with genetic disorders. The nucleotide sequences provided herein can be mapped to a chromosome and specific regions of a chromosome using known techniques, such as in situ hybridization, linkage analysis against known chromosomal markers, and hybridization screening with libraries.

Antibodies

In some methods, receptor proteins or ligand proteins that affect signaling in apoptotic cell-responding B220⁻ dendritic cell can be used to generate polyclonal and monoclonal antibodies to receptor or ligand proteins, which are useful as described herein. A number of immunogens are used to produce antibodies that specifically bind receptor or ligand polypeptides. Full-length receptor or ligand polypeptides are suitable immunogens. Typically, the immunogen of interest is a peptide of at least about 3 amino acids, more typically the peptide is at least 5 amino acids in length, the fragment is at least 10 amino acids in length and typically the fragment is at least 15 amino acids in length. The peptides can be coupled to a carrier protein (e.g., as a fusion protein), or are recombinantly expressed in an immunization vector. Antigenic determinants on peptides to which antibodies bind are typically 3 to 10 amino acids in length. Naturally occurring polypeptides are also used either in pure or impure form. Recombinant polypeptides are expressed in eukaryotic or prokaryotic cells and purified using standard techniques. The polypeptide, or a synthetic version thereof, is then injected into an animal capable of producing antibodies. Either monoclonal or polyclonal antibodies can be generated for subsequent use in immunoassays to measure the presence and quantity of the polypeptide.

These antibodies find use in a number of applications. For example, the receptor or ligand antibodies can be coupled to standard affinity chromatography columns and used to purify receptor or ligand proteins as further described below. The antibodies can also be used as blocking polypeptides, as outlined above, since they will specifically bind to the receptor or ligand protein to activate or inhibit signaling in apoptotic cell-responding B220⁻ dendritic cell.

The anti-receptor or anti-ligand protein antibodies can comprise polyclonal antibodies. Methods for producing polyclonal antibodies are known to those of skill in the art. In brief, an immunogen, for example, a purified polypeptide, a polypeptide coupled to an appropriate carrier (e.g., GST and keyhole limpet hemocyanin), or a polypeptide incorporated into an immunization vector such as a recombinant vaccinia virus (see, U.S. Pat. No. 4,722,848) is mixed with an adjuvant and animals are immunized with the mixture. The animal's immune response to the immunogen preparation is monitored by taking test bleeds and determining the titer of reactivity to the polypeptide of interest. When appropriately high titers of antibody to the immunogen are obtained, blood is collected from the animal and antisera are prepared. Further fractionation of the antisera to enrich for antibodies reactive to the polypeptide is performed where desired. See, e.g., Coligan, Current Protocols in Immunology, Wiley/Greene, N.Y., 1991; and Harlow and Lane, supra, each incorporated herein by reference in their entirety.

Antibodies, including binding fragments and single chain recombinant versions thereof, against predetermined fragments of receptor or ligand proteins are raised by immunizing animals, e.g., with conjugates of the fragments with carrier proteins as described above.

The anti-receptor or anti-ligand protein antibodies can, alternatively, be monoclonal antibodies. The monoclonal antibodies are prepared from cells secreting the desired antibody. These antibodies are screened for binding to normal or modified polypeptides, or screened for agonistic or antagonistic activity, e.g., activity mediated through the receptor proteins or ligand proteins. In some instances, it is desirable to prepare monoclonal antibodies from various mammalian hosts, such as mice, rodents, primates, and humans. Description of techniques for preparing such monoclonal antibodies are found in, e.g., Stites et al. (eds.) Basic and Clinical Immunology (4th ed.) Lange Medical Publications, Los Altos, Calif., and references cited therein; Harlow and Lane, Supra; Goding, 1986; Monoclonal Antibodies. Principles and Practice (2d ed.) Academic Press, New York, N.Y.; and Kohler et al., Nature 256:495-497, 1975, each incorporated herein by reference in their entirety.

The immunizing agent will typically include the receptor polypeptide or ligand polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, 1986, pp. 59-103, incorporated herein by reference in its entirety). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Immortalized cell lines are those that fuse efficiently, support stable high level expression of antibody by the selected antibody-producing cells, and are sensitive to a medium such as HAT medium. More immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol. 133:3001, 1984; Brodeur et al., Monoclonal Antibody Production Techniques and Applications, Marcel Dekker, Inc., New York, 1987, pp. 51-63, each incorporated herein by reference in their entirety).

Other suitable techniques involve selection of libraries of recombinant antibodies in phage or similar vectors. See, Huse et al., Science 246:1275-1281, 1989; and Ward et al., Nature 341:544-546, 1989, each incorporated herein by reference in their entirety.

Also, recombinant immunoglobulins can be produced. For example, see, U.S. Pat. No. 4,816,567; and Queen et al., Proc. Natl Acad. Sci. 86:10029-10033, 1989, each incorporated herein by reference in their entirety.

Antibodies are also used for affinity chromatography in isolating receptor or ligand proteins. Columns are prepared, e.g., with the antibodies linked to a solid support, e.g., particles, such as agarose, Sephadex, or the like, where a cell lysate is passed through the column, washed, and treated with increasing concentrations of a mild denaturant, whereby purified receptor or ligand polypeptides are released.

A further approach for isolating DNA sequences which encode a human monoclonal antibody or a binding fragment thereof is by screening a DNA library from human B cells according to the general protocol outlined by Huse et al., Science 246:1275-1281, 1989, incorporated herein by reference in its entirety, and then cloning and amplifying the sequences which encode the antibody (or binding fragment) of the desired specificity. Such B cells can be obtained from a human immunized with the desired antigen, fragments, longer polypeptides containing the antigen or fragments or anti-idiotypic antibodies. Optionally, such B cells are obtained from an individual who has not been exposed to the antigen. B cell can also be obtained from transgenic non-human animals expressing human immunoglobulin sequences. The transgenic non-human animals can be immunized with an antigen or collection of antigens. The animals can also be unimmunized. B cell mRNA sequences encoding human antibodies are used to generate cDNA using reverse transcriptase. The V region encoding segments of the cDNA sequences are then cloned into a DNA vector that directs expression of the antibody V regions. Typically the V region sequences are specifically amplified by PCR prior to cloning. Also typically, the V region sequences are cloned into a site within the DNA vector that is constructed so that the V region is expressed as a fusion protein. Examples of such fusion proteins include m13 coliphage gene 3 and gene 8 fusion proteins. The collection of cloned V region sequences is then used to generate an expression library of antibody V regions. To generate an expression library, the DNA vector comprising the cloned V region sequences is used to transform eukaryotic or prokaryotic host cells. In addition to V regions, the vector can optionally encode all or part of a viral genome, and can comprise viral packaging sequences. In some cases the vector does not comprise an entire virus genome, and the vector is then used together with a helper virus or helper virus DNA sequences. The expressed antibody V regions are found in, or on the surface of, transformed cells or virus particles from the transformed cells. This expression library, comprising the cells or virus particles, is then used to identify V region sequences that encode antibodies, or antibody fragments reactive with predetermined antigens. To identify these V region sequences, the expression library is screened or selected for reactivity of the expressed V regions with the predetermined antigens. The cells or virus particles comprising the cloned V region sequences, and having the expressed V regions, are screened or selected by a method that identifies or enriches for cells or virus particles that have V regions reactive (e.g., binding association or catalytic activity) with a predetermined antigen. For example, radioactive or fluorescent labeled antigen that then binds to expressed V regions can be detected and used to identify or sort cells or virus particles. Antigen bound to a solid matrix or bead can also be used to select cells or virus particles having reactive V regions on the surface. The V region sequences thus identified from the expression library can then be used to direct expression, in a transformed host cell, of an antibody or fragment thereof, having reactivity with the predetermined antigen.

The protocol described by Huse is rendered more efficient in combination with phage-display technology. See, e.g., Dower et al., WO 91/17271; McCafferty et al., WO 92/01047; and U.S. Pat. Nos. 5,871,907; 5,858,657; 5,837,242; 5,733,743; and 5,565,332, each incorporated herein by reference in their entirety. In these methods, libraries of phage are produced in which members (display packages) display different antibodies on their outer surfaces. Antibodies are usually displayed as Fv or Fab fragments. Phage displaying antibodies with a desired specificity can be selected by affinity enrichment to the antigen or fragment thereof. Phage display combined with immunized transgenic non-human animals expressing human immunoglobulin genes can be used to obtain antigen specific antibodies even when the immune response to the antigen is weak. In a variation of the phage-display method, human antibodies having the binding specificity of a selected murine antibody can be produced. See, for example, WO 92/20791, incorporated herein by reference in its entirety.

An alternative approach is the generation of humanized immunoglobulins by linking the CDR regions of non-human antibodies to human constant regions by recombinant DNA techniques. See U.S. Pat. No. 5,585,089, incorporated herein by reference in its entirety. Humanized forms of non-human (e.g., murine) antibodies are immunoglobulins, immunoglobulin chains or fragments thereof (such as Fᵥ, F_{ab}, F_{ab′}, F_{ab2} or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fᵥ framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an F_{c} region, typically that of a human immunoglobulin. See Jones et al., Nature 321: 522-525, 1986; Riechmann et al., Nature 332: 323-329, 1988; and Presta, Curr. Op. Struct. Biol., 2:593-596, 1992, each incorporated herein by reference in their entirety. See, Queen et al., Proc. Natl. Acad. Sci. U.S.A. 86:10029-10033, 1989; and WO 90/07861; U.S. Pat. No. 5,693,762; U.S. Pat. No. 5,693,761; U.S. Pat. No. 5,585,089; U.S. Pat. No. 5,530,101; and U.S. Pat. No. 5,225,539, each incorporated herein by reference in their entirety.

Bispecific antibodies are monoclonal, typically human or humanized, antibodies that have binding specificities for at least two different antigens. In the present case, one of the binding specificities is for the receptor protein or ligand protein, the other one is for any other antigen, and for a cell-surface protein or receptor or receptor subunit. Milstein and Cuello, Nature 305:537-539, 1983.

The anti-receptor or anti-ligand protein antibodies have various utilities. For example, anti- receptor or anti-ligand protein antibodies can be used in diagnostic assays for a receptor or ligand protein, e.g., detecting its expression in specific cells, tissues, or serum. Various diagnostic assay techniques can be used, such as competitive binding assays, direct or indirect sandwich assays and immunoprecipitation assays conducted in either heterogeneous or homogeneous phases. Zola, 1987, Monoclonal Antibodies: A Manual of Techniques 147-158. The antibodies used in the diagnostic assays can be labeled with a detectable moiety. The detectable moiety should be capable of producing, either directly or indirectly, a detectable signal. For example, the detectable moiety can be a radioisotope, such as 3H, 14C, ³²p, 35S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety can be employed, including those methods described by Hunter et al., Nature 144:945, 1962; David et al., Biochemistry 13:1014, 1974; Pain et al., J. Immunol. Meth. 40:219, 1981; and Nygren, J. Histochem. and Cytochem. 30:407, 1982. Each citation is incorporated herein by reference in their entirety.

Anti-receptor or anti-ligand protein antibodies also are useful for the affinity purification of receptor or ligand protein from recombinant cell culture or natural sources. In this process, the antibodies against receptor or ligand protein are immobilized on a suitable support, such a Sephadex resin or filter paper, using methods well known in the art. The immobilized antibody then is contacted with a sample containing the protein to be purified, and thereafter the support is washed with a suitable solvent that will remove substantially all the material in the sample except the receptor or ligand protein, which is bound to the immobilized antibody. Finally, the support is washed with another suitable solvent that will release the protein from the antibody.

Pharmaceutical Compositions and Methods of Administration

The anti-receptor or anti-ligand antibodies that act as agonists or antagonists of signaling in apoptotic cell-responding B220⁻ dendritic cell can also be used in treatment. In some methods, the genes encoding the antibodies are provided, such that the antibodies bind to and modulate signaling in apoptotic cell-responding B220⁻ dendritic cell. In other methods, a therapeutically effective amount of a receptor protein or a ligand protein, agonist or antagonist is administered to a patient. Further therapeutic treatments include pharmaceutical compositions that are agonists or antagonists of signaling in apoptotic cell-responding B220⁻ dendritic cell. For example, the agonist or antagonist includes, but is not limited to, a polypeptide, nucleic acid, small molecule, antisense oligonucleotide, ribozyme, RNAi construct, siRNA, shRNA, or antibody. A “therapeutically effective amount”, “pharmacologically acceptable dose”, “pharmacologically acceptable amount” means that a sufficient amount of an immunosuppressive agent or combination of agents is present to achieve a desired result, e.g., preventing, delaying, inhibiting or reversing a symptom of a disease or disorder or the progression of disease or disorder when administered in an appropriate regime.

Pharmaceutically acceptable carriers are determined in part by the particular composition being administered, as well as by the particular method used to administer the composition. Accordingly, there is a wide variety of suitable formulations of pharmaceutical compositions (see, e.g., Alfonso R Gennaro (ed), 2003, Remington. The Science and Practice of Pharmacy, (Formerly Remington's Pharmaceutical Sciences) 20th ed., incorporated herein by reference in its entirety). The pharmaceutical compositions generally comprise a receptor or a ligand protein agonist or antagonist, e.g., a polypeptide, nucleic acid, small molecule, antisense oligonucleotide, ribozyme, RNAi construct, siRNA, shRNA, or antibody, in a form suitable for administration to a patient. The pharmaceutical compositions are generally formulated as sterile, substantially isotonic and in full compliance with all Good Manufacturing Practice (GMP) regulations of the U.S. Food and Drug Administration.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art.

In some methods, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like, particularly the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The nucleic acids, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the packaged nucleic acid with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Compositions can be administered, for example, by intravenous infusion, orally, topically, intranasally, intraperitoneally, intravesically or intrathecally. Formulations for injection can be presented in unit dosage form, e.g., in ampules or in multidose containers, with an added preservative.

Injection solutions and suspensions can be prepared from sterile powders, granules, and tablets of the kind previously described. Cells transduced by the packaged nucleic acid as described above in the context of ex vivo therapy can also be administered intravenously or parenterally as described above.

The dose administered to a patient should be sufficient to effect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

Effective doses of the pharmaceutical compositions (e.g., monoclonal antibodies, human sequence antibodies, human antibodies, multispecific and bispecific molecules, small chemical molecules, nucleic acid compositions, e.g., antisense oligonucleotides, double stranded RNA oligonucleotides (RNAi; shRNA, si RNA), or DNA oligonucleotides or vectors containing nucleotide sequences encoding for the transcription of shRNA molecules) that inhibit tissue factor signaling, or other inhibitors of tissue factor, e.g., small molecule inhibitors, for the treatment of neoplastic disease or inflammatory disease, described herein vary depending upon many different-factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

For administration with an therapeutic antibody or small molecule composition, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. In some methods, two or more therapeutic antibody or small molecule compositions with different binding target specificities are administered simultaneously, in which case the dosage of each therapeutic antibody or small molecule composition administered falls within the ranges indicated. A therapeutic antibody or small molecule composition is usually administered on multiple occasions. Intervals between single dosages can be weekly, monthly or yearly. Intervals can also be irregular as indicated by measuring blood levels of therapeutic antibody or small molecule composition in the patient. In some methods, dosage is adjusted to achieve a plasma antibody or small molecule composition concentration of 1-1000 μg/ml and in some methods 25-300 μg/ml. Alternatively, an antibody or small molecule composition can be administered as a sustained release formulation, in which case less frequent administration is required. Dosage and frequency vary depending on the half-life of the therapeutic antibody or small molecule composition in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Doses for therapeutic antibody or small molecule composition range from about 10 ng to 1 g, 100 ng to 100 mg, 1 μg to 10 mg, or 30-300 μg per patient.

In determining the effective amount of the vector to be administered in the treatment or prophylaxis of conditions resulting from expression of the receptor or ligand proteins or analogs thereof, or antibodies to receptor or ligand proteins, of the methods and compositions, the physician evaluates circulating plasma levels of the vector, vector toxicities, progression of the disease, and the production of anti-vector antibodies. In general, the dose equivalent of a naked nucleic acid from a vector is from about 1 μg to 100 μg for a typical 70 kilogram patient, and doses of vectors which include a retroviral particle are calculated to yield an equivalent amount of therapeutic nucleic acid.

For administration, inhibitors and transduced cells can be administered at a rate determined by the LD₅₀ of the inhibitor, vector, or transduced cell type, and the side-effects of the inhibitor, vector or cell type at various concentrations, as applied to the mass and overall health of the patient. Administration can be accomplished via single or divided doses.

Transduced cells are prepared for reinfusion according to established methods. See Abrahamsen et al., J. Clin. Apheresis 6:48-53, 1991; Carter et al., J. Clin. Arpheresis 4:113-117, 1998; Aebersold et al., J. Immunol. Meth. 112:1-7, 1998; Muul et al., J. Immunol. Methods, 101: 171-181, 1987; and Carter et al., Transfusion 27: 362-365, 1987, each incorporated herein by reference in their entirety. After a period of about 2-4 weeks in culture, the cells should number between 1×10⁸ and 1×10¹². In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent.

Kits

Agonists or antagonists or their homologs are useful tools for examining expression and regulation of signaling in apoptotic cell-responding B220⁻ dendritic cell. Reagents that specifically hybridize to nucleic acids encoding receptor proteins or ligand proteins (including probes and primers of the proteins), and reagents that specifically bind to the proteins, e.g., antibodies, are used to examine expression and regulation.

Nucleic acid assays for the presence of agonists proteins or antagonist proteins in a sample include numerous techniques are known to those skilled in the art, such as Southern analysis, northern analysis, dot blots, RNase protection, S1 analysis, amplification techniques such as PCR and LCR, high density oligonucleotide array analysis, and in situ hybridization. In in situ hybridization, for example, the target nucleic acid is liberated from its cellular surroundings in such as to be available for hybridization within the cell while preserving the cellular morphology for subsequent interpretation and analysis. The following articles provide an overview of the art of in situ hybridization: Singer et al., Biotechniques 4:230-250, 1986; Haase et al., Methods in Virology, VII: 189-226, 1984; and Nucleic Acid Hybridization: A Practical Approach (Hames et al., eds. 1987), each incorporated herein by reference in their entirety. In addition, receptor proteins or ligand proteins can be detected with the various immunoassay techniques described above. The test sample is typically compared to both a positive control (e.g., a sample expressing recombinant receptor protein or ligand protein) and a negative control.

Kits are provided for screening agonists or antagonists which affect signaling in apoptotic cell-responding B220⁻ dendritic cell. Such kits can be prepared from readily available materials and reagents are provided. For example, such kits can comprise any one or more of the following materials: the receptor proteins or ligand proteins, agonists, or antagonists, reaction tubes, and instructions for testing the activities of receptor protein genes or ligand protein genes. A wide variety of kits and components can be prepared depending upon the intended user of the kit and the particular needs of the user. For example, the kit can be tailored for in vitro or in vivo assays for measuring the activity of receptor proteins or ligand proteins proteins or modulators of signaling in apoptotic cell-responding B220⁻ dendritic cell.

Kits comprising probe arrays as described above are provided. Optional additional components of the kit include, for example, other restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions.

Usually, the kits also contain instructions for carrying out the methods.

Other embodiments and uses will be apparent to one skilled in the art in light of the present disclosures.

### Exemplary Embodiments

### EXAMPLE 1

CTL Responses Induced by Cell Death

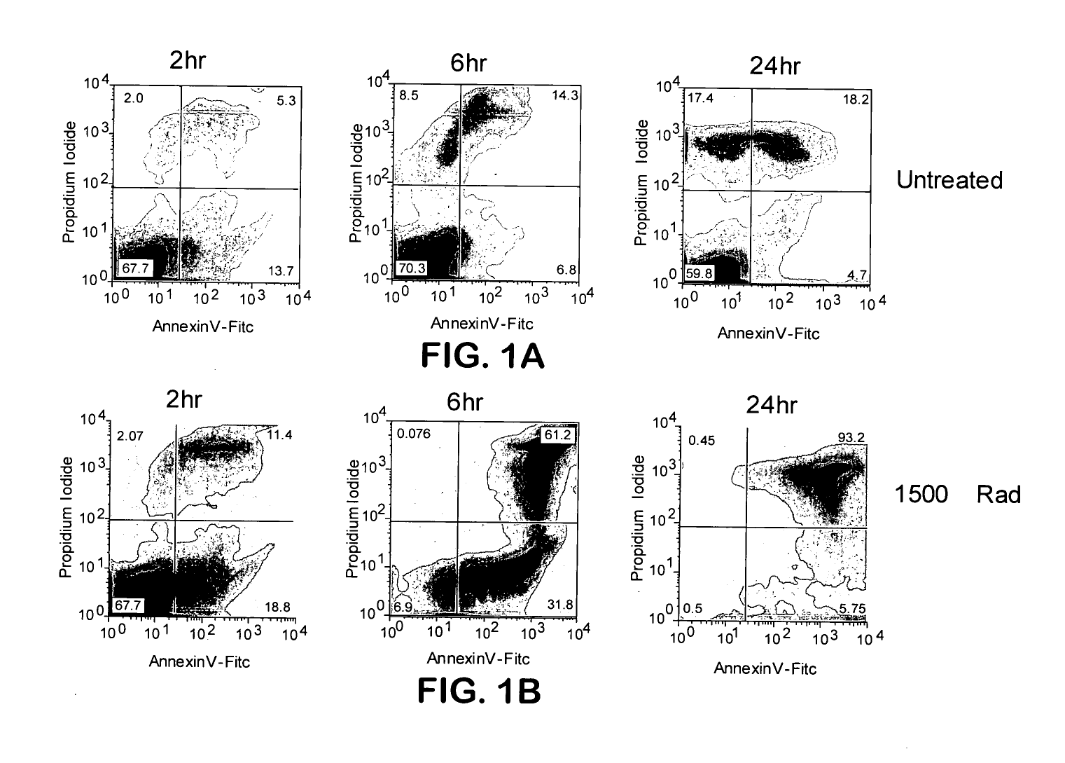

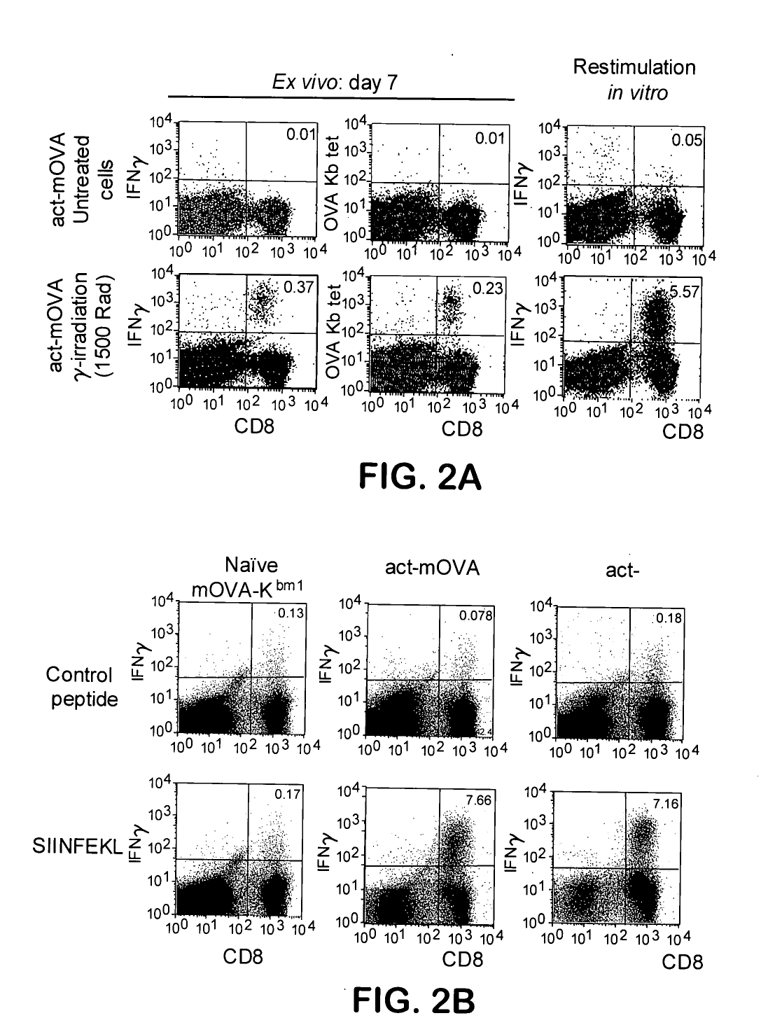

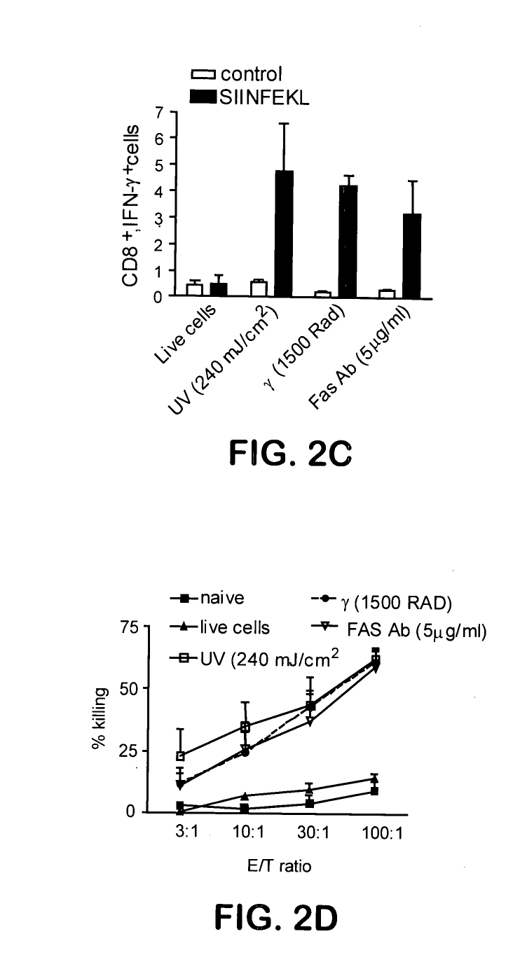

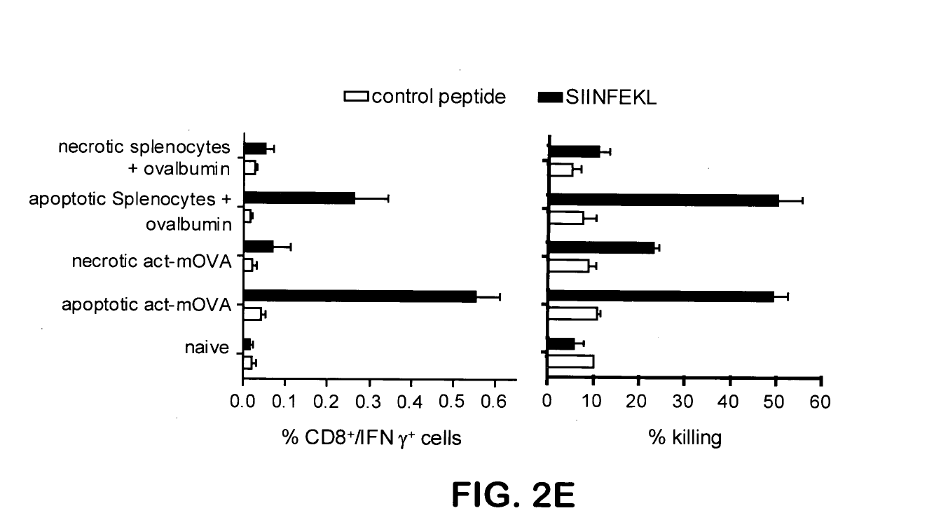

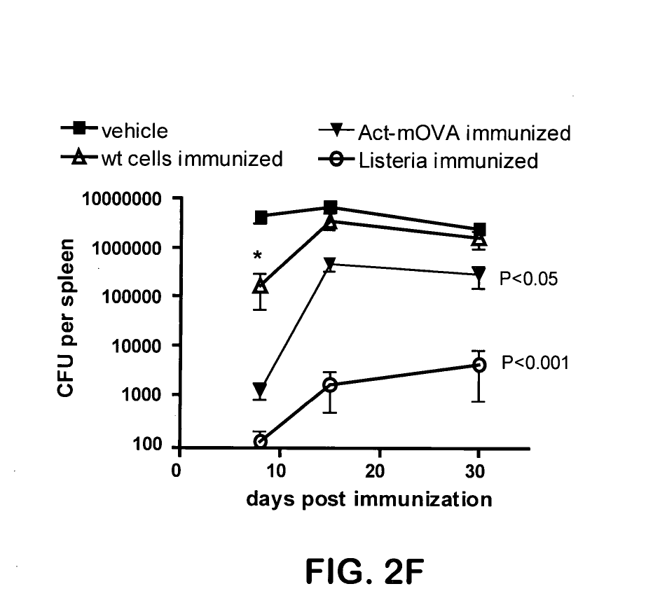

C57BL/6 mice were immunized with 10 million syngeneic cells transgenic for an actin promoter-driven, membrane-associated form of ovalbumin (act-mOVA cells (Ehst et al., Am. J. Transplant. 3: 1355-1362, 2003)). The transgenic cells were either treated with γ-irradiation (1500 rad) to induce apoptosis or left untreated prior to injection (FIG. 1). After 8 days, splenocytes were isolated from each mouse, and restimulated using OVA₂₅₇₋₂₆₄ peptide (5 μg/ml). The frequency of antigen-specific CD8⁺ T cells was determined by measuring intracellular IFN-γ production as well as specific binding of OVA-H2^{kb} tetramers, as previously described. Hoebe et al., Nat. Immunol. 4: 1223-1229, 2003. In parallel, cells were restimulated for 6 days to address their cytolytic activity and their capacity for secondary expansion, a hallmark of T cell memory. While immunization with apoptotic cells resulted in strong CD8⁺/IFN-γ⁺ response, immunization with non-irradiated act-mOVA cells did not (FIG. 2a). To exclude direct priming by apoptotic cells, mice were immunized with either irradiated act-mOVA splenocytes or act-mOVA splenocytes on a K^{bm1} background avoiding direct priming via MHC class I. Both immunizations of mice with γ-irradiated (1500 rad) act-mOVA or act-mOVA-K^{bm1} splenocytes induced strong CD8⁺ T cell responses as measured by the number of CD8⁺/IFN-γ⁺ cells after restimulation with the OVA₂₅₇₋₂₆₄ peptide (FIG. 2b). To test whether the response could be induced by different apoptotic stimuli, mice were immunized with act-mOVA splenocytes that were either UV-radiated (254 nm wavelength; 240 mJ/cm²) or treated with Fas-activating antibody (5 μg/ml). Each protocol resulted in the development of strong cytotoxic T-lymphocyte (CTL) responses with a high frequency of antigen-specific CD8⁺/IFN-γ⁺ cells and efficient killing of target cells (FIGS. 2c,2d). In addition, immunization of mice with γ-irradiated wildtype splenocytes pulsed with ovalbumin, resulted in responses similar to those observed for irradiated act-mOVA cells (FIG. 2e), whereas, immunization with necrotic act-mOVA cells or necrotic wildtype splenocytes pulsed with ovalbumin and treated with multiple cycles of freeze/thawing resulted in a much weaker CD8⁺ T cell response (FIG. 2e), indicating that necrotic cells (as opposed to apoptotic cells) are less efficient in inducing a CTL response.

To assess whether this route of immunization could initiate long term protection against pathogenic infections, wildtype C57BL/6 were treated subcutaneously with either PBS, irradiated splenocytes from wildtype or act-mOVA cells, or with 1×10³ OVA-expressing Listeria monocytogenes. At various days post immunization, mice were challenged with 1×10⁵ OVA-expressing L. monocytogenes i.v. to determine their ability to clear the bacteria. Mice immunized with irradiated act-mOVA cells or OVA-expressing L. monocytogenes showed significantly reduced titers upon L. monocytogenes challenge up to 30 days after immunization (FIG. 2f). Surprisingly, compared to vehicle treated mice, the group immunized with wildtype irradiated splenocytes showed significantly reduced titers at day 8 (P<0.001) (FIG. 2f), suggesting irradiated syngeneic cells are recognized and induce an inflammatory response leading to anti-bacterial protection.

FIG. 1 shows that percentage of cells undergoing apoptosis as measured by annexin V and propidium Iodide in isolated splenocytes either left untreated or treated with γ-irradiation (1500 rad) during a 24 hr incubation period.

FIG. 2 shows that apoptotic but not live act-mOVA cells induce strong CTL responses. (a) Comparison of CD8-specific T cell responses after restimulation ex vivo or following secondary expansion in vitro with OVA peptide in wildtype mice immunized with untreated act-mOVA cells or treated with act-mOVA cells rendered apoptotic through exposure to γ-irradiation (1500 rad). (b) Comparison of CD8-specific T cell responses after secondary expansion and restimulation of splenocytes in vitro from naive mice or mice immunized with γ-irradiated (1500 rad) act-mOVA or act-mOVA-K^{bm-1} splenocytes (c,d) CD8⁺ T cell responses following secondary expansion in vitro after immunization of mice with act-mOVA cells rendered apoptotic through treatment with γ-irradiation (1500 rad), UV-radiation (240 mJ/cm²) or treatment with Fas-activating antibody (5 μg/ml). (e) CD8⁺ T cell responses ex vivo in mice immunized with γ-irradiated act-mOVA, γ-irradiated wildtype splenocytes pulsed with OVA protein (both 1500 rad), or with act-mOVA cells submitted to multiple freeze/thaw cycles. (E:T ratio: 30:1) (f) Splenic titers of Listeria monocytogenes after bacterial challenge with 1×10⁵ CFU i.v. at day 8, 15 and 30 post immunization. Mice were immunized with either PBS, γ-irradiated wildtype or act-mOVA splenocytes (10 million/mouse) or 1×10³ CFU L. monocytogenes. (Values represent mean±SEM; n=5 for each group, *=P<0.01 difference of L. monocytogenes titers between mice treated with vehicle and mice immunized with γ-irradiated Wt cells at day 8 post immunization.

### EXAMPLE 2

Apoptotic Cells Induce Type I IFN, a Key Mediator of In Vivo CTL Responses

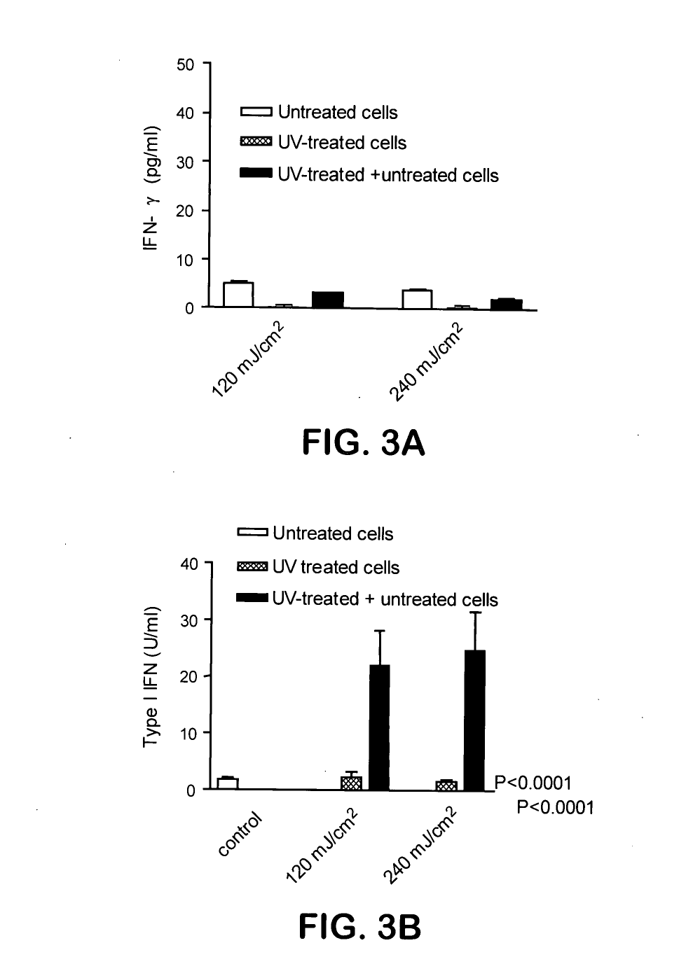

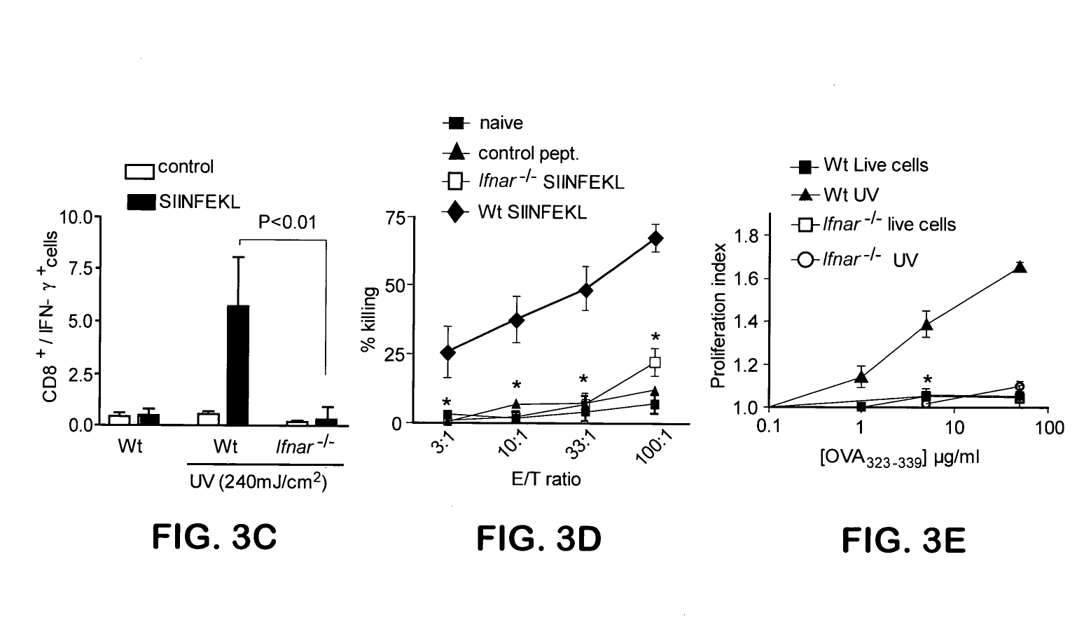

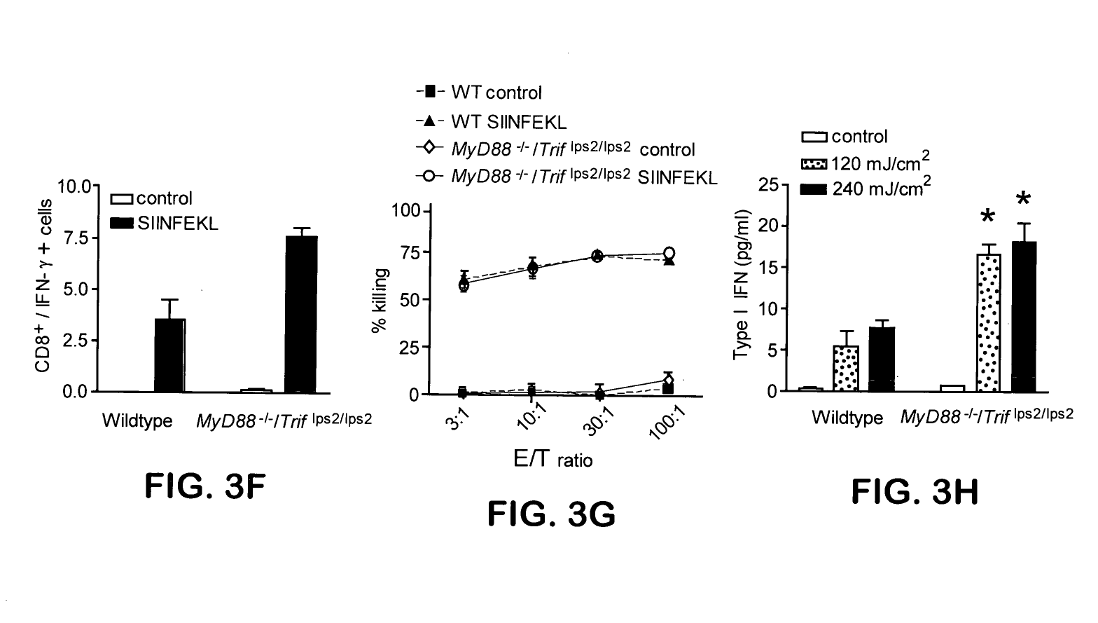

The mechanism of apoptotic cell sensing was investigated by dividing total splenocytes into two aliquots, one exposed to UV-radiation to induce apoptosis and the other untreated. The two populations were then cultured alone or as a mixture for 48 hrs, and medium was collected and tested for cytokine production. No significant tumor necrosis factor-α (TNF-α) or interferon (IFN)-γ production (FIG. 3a) was detected in any of the cultures, but robust production of type I IFN was observed after 24 hr incubation when UV-treated and untreated cells were co-cultured (FIG. 3b). It was concluded that responder cells in the untreated population sensed apoptotic cells in the UV-treated population and produced type I IFN.

The importance of type I IFN in the generation of CTL responses in vivo was examined. When wildtype and IFN type I receptor knockout mice (Ifnar^{-/-}) were immunized with apoptotic act-mOVA cells, the latter showed a marked reduction in the percentage of CD8⁺/IFN-γ⁺ cells (FIG. 3c ) as well as the ability to kill OVA-expressing target cells (FIG. 3d), and failed to proliferate when exposed to CD4-specific OVA peptides (FIG. 3e). These results indicate that type I IFN is a key mediator of immune responses induced by apoptotic cells.

FIG. 3 shows that type I IFN is a key mediator of apoptotic cell-induced CTL responses and is induced via a TLR-independent pathway. Splenocytes were untreated or treated with 120 or 240 mJ/cm² of UV-radiation. Subsequently, cells were cultured separately as monocultures or as co-cultures containing UV-radiated and untreated cells. At various time points, supernatants were collected and analyzed for the presence of (a) IFN-γ (24 hrs of incubation) or (b) type I IFN (24 hrs of incubation), as described in methods. CD8⁺ (c,d) or CD4⁺ T cell (e) responses in naive, or wildtype and Ifnar^{-l-} knockout mice immunized with untreated or UV-treated act-mOVA cells (240 mJ/cm²). (*=P<0.001 between Wt and Ifnar^{-l-}) (f,g) CD8⁺ T cell responses in wildtype and MyD88^{-l-}; Trif^{lps2/lps2} mice after immunization with UV-treated act-mOVA splenocytes (240 mJ/cm²). (Graphs represent mean±SEM; 4 mice per group). (h) Induction of type I IFN in wildtype and MyD88^{-l-}; Trif^{lps2/lps2} splenocytes after 24 hr exposure to non-treated or UV-treated cells (120 or 240 mJ/cm²). Values represent mean values±SEM of 3 independent experiments.

### EXAMPLE 3

Cell-Death-Induced CTL Responses Do Not Depend Upon TLR Signaling

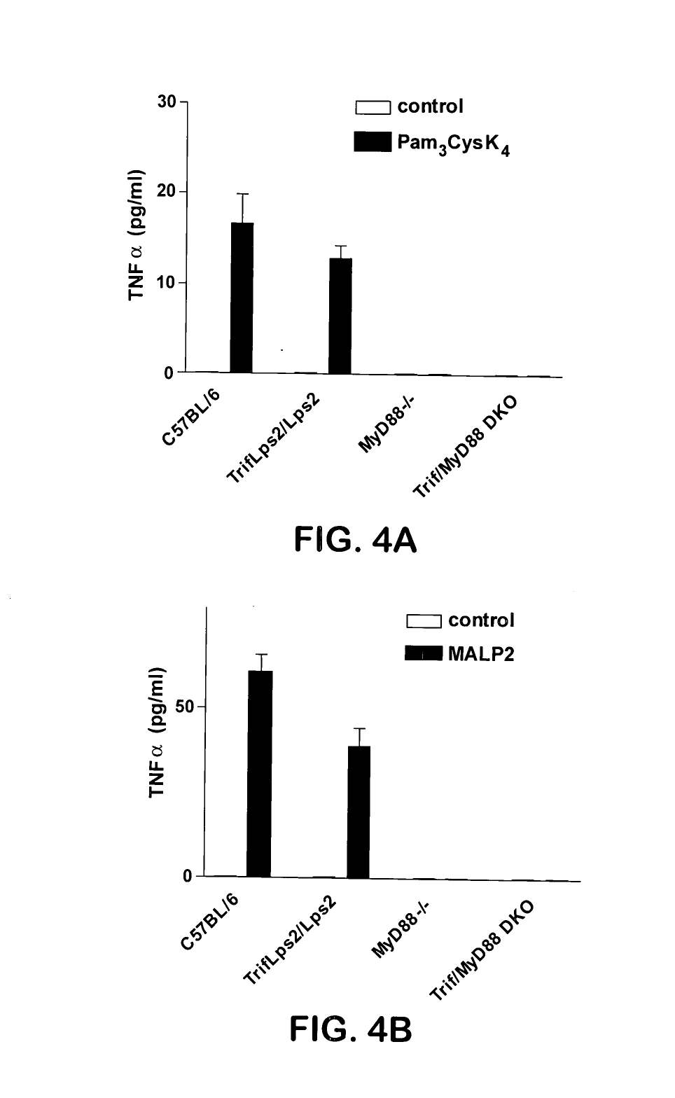

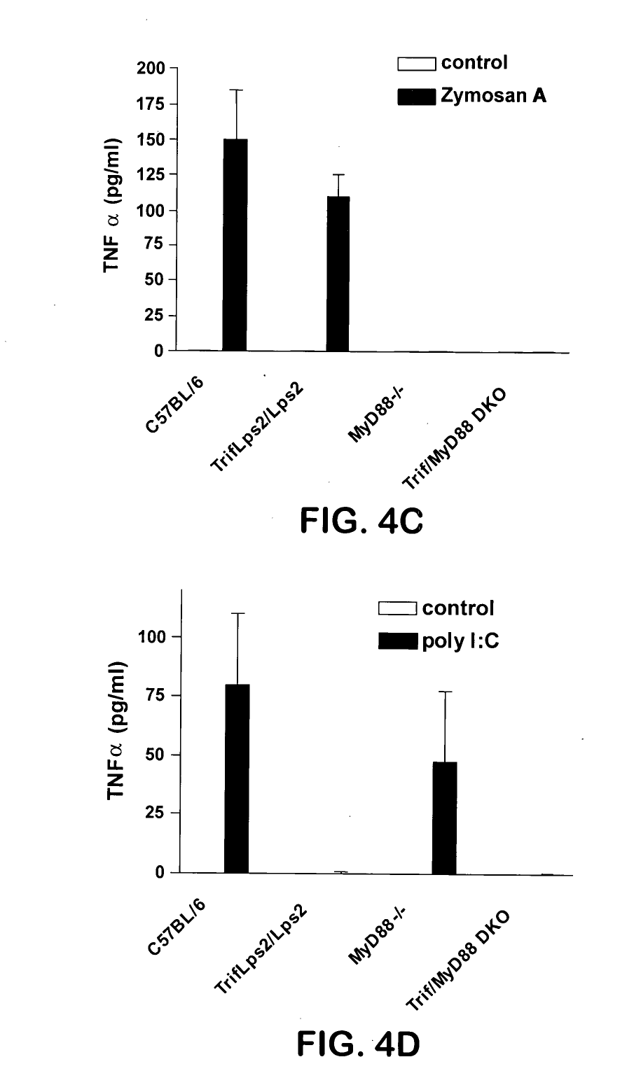

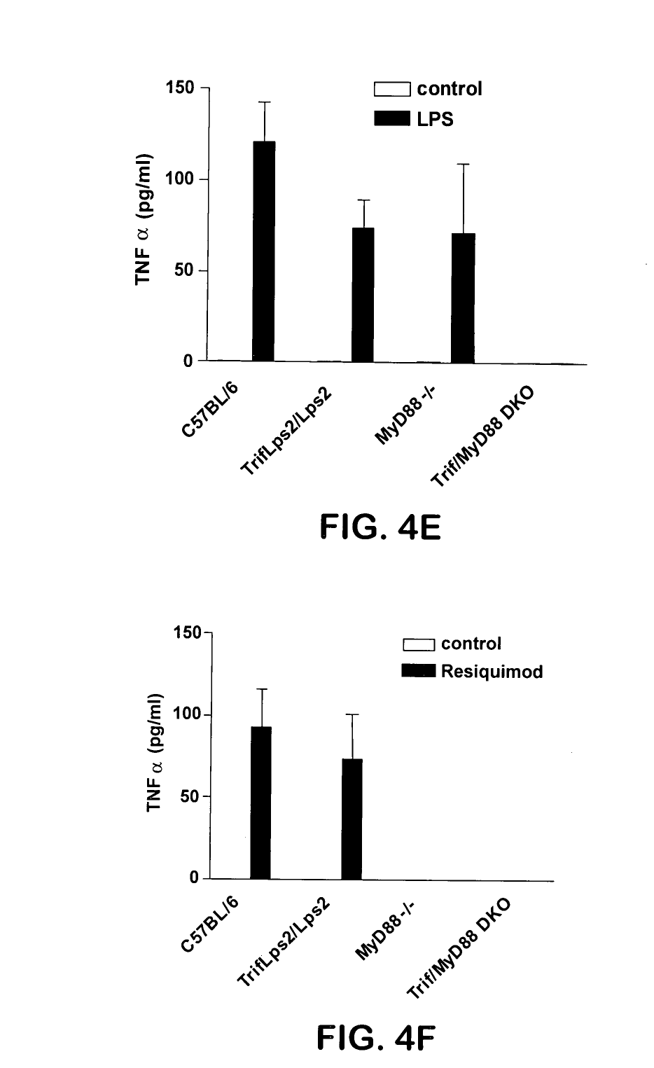

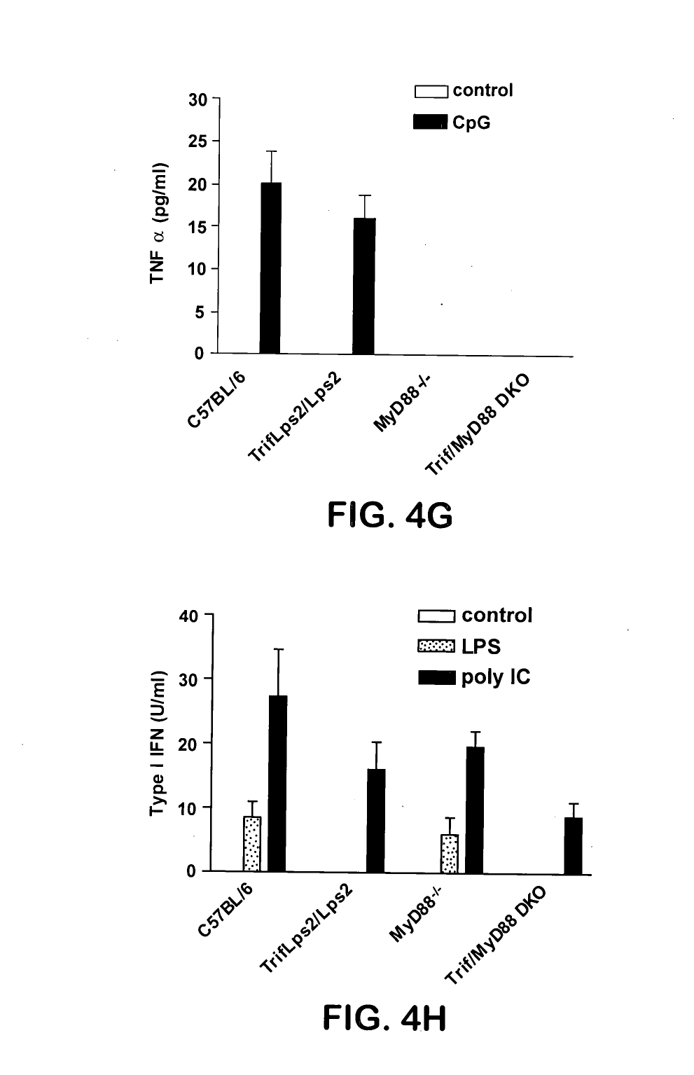

Since it was suggested that TLRs are essential for recognition of molecular constituents released from injured tissues or apoptotic cells of the host, responses to apoptotic cells were studied in mice with homozygous mutations in both MyD88 and Trif genes (MyD88^{-l-}; Trif^{lps2/lps}² mice (Hoebe et al., Nature 424: 743-748, 2003)). Seong et al., Nat. Rev. Immunol. 4: 469-478, 2004. TLR-mediated macrophage responses to all known TLR ligands are abolished in these mice except for dsRNA, which is known to signal via a TLR-independent pathway and elicits type I IFN production, but not TNF-α production (FIG. 4a-4h). Hoebe et al., Nat. Immunol. 4: 1223-1229, 2003.

In MyD88^{-l-}; Trif^{lps2/lps2} mice, the number of CD8⁺/IFN-γ⁺ T cells generated in response to immunization with UV-treated act-mOVA cells appeared to be increased compared to the number observed in wildtype mice (FIG. 3f), and both wildtype and MyD88^{-l-}; Trif^{lps2/lps2} mice supported the generation of equivalent cytotoxic activity against OVA-expressing target cells (FIG. 3g). Finally, type I IFN production was slightly increased in splenocyte cultures from mice lacking TLR signaling compared to wildtype mice when exposed to UV-treated (240 mJ/cm²) splenocytes (FIG. 3h). These results effectively challenge the assumption that TLRs are involved in cell death-induced immune responses, and instead suggest the involvement of a previously undescribed TLR-independent pathway leading to type I IFN production.

FIG. 4 shows that abrogated TLR signaling in MyD88^{-l-}; Trif^{lps2/lps2} double-deficient mice. Peritoneal macrophages derived from wildtype C57BL/6, Trif mutant (Trif^{lps2/lps2}), MyD88^{-l-}; and MyD88^{-l-}; Trif^{lps2/lps2} doubly deficient mice were activa TLR-specific stimuli. After 4 hours incubation, supernatant was collected and analyzed for the presence of TNF-α using the L929 bioassay (a-g) or type I IFN using a ISRE luciferase reporter L929 cell line (h).

### EXAMPLE 4

Flt3L-Derived B220⁻, but not GM-CSF-Derived DCs, Respond to Apoptotic Cells

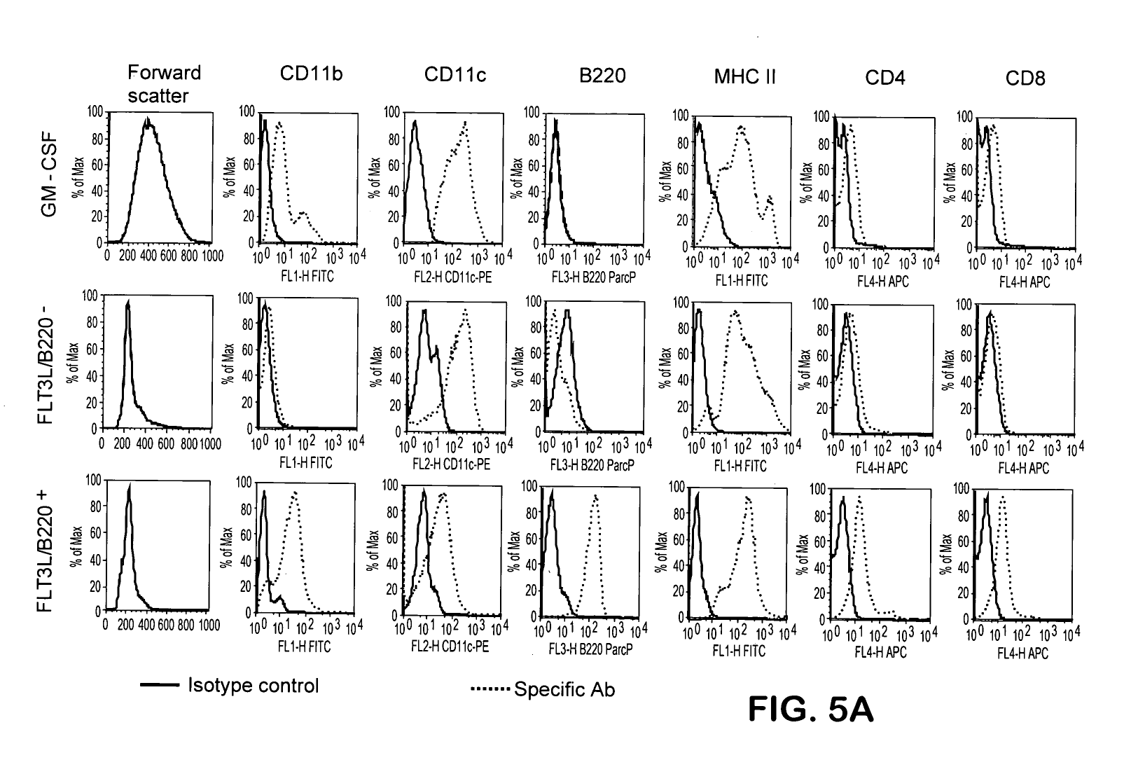

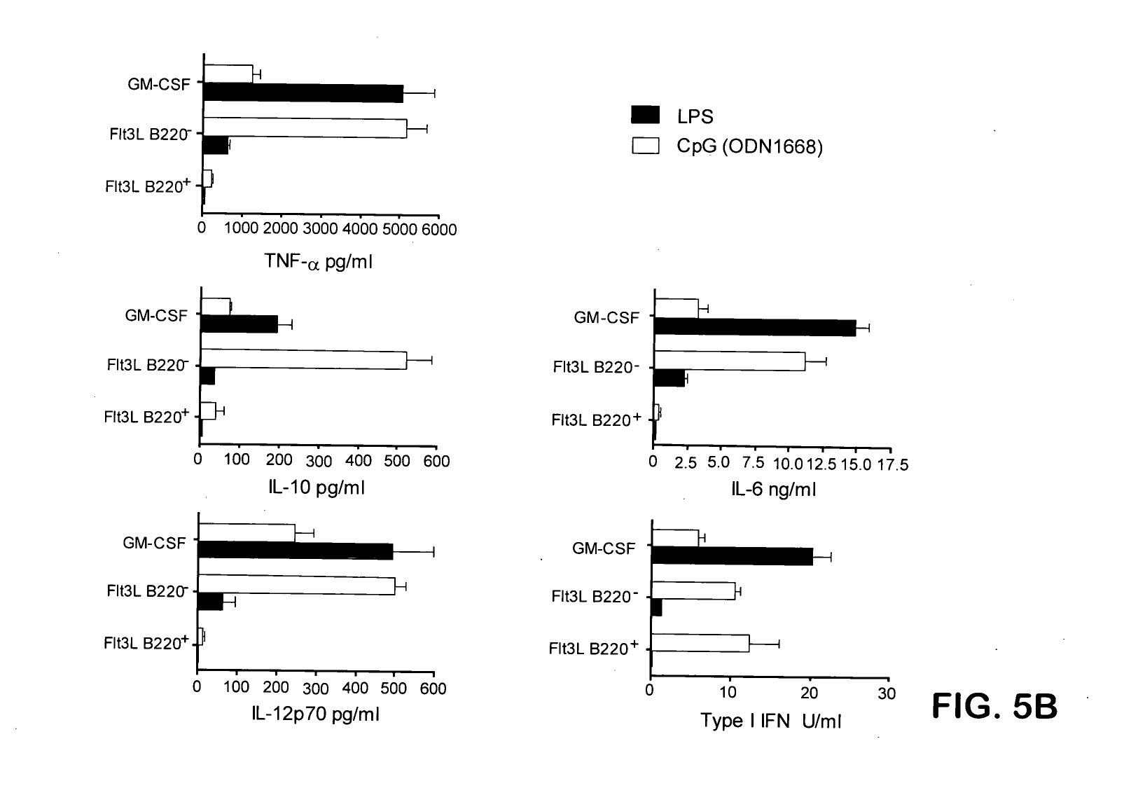

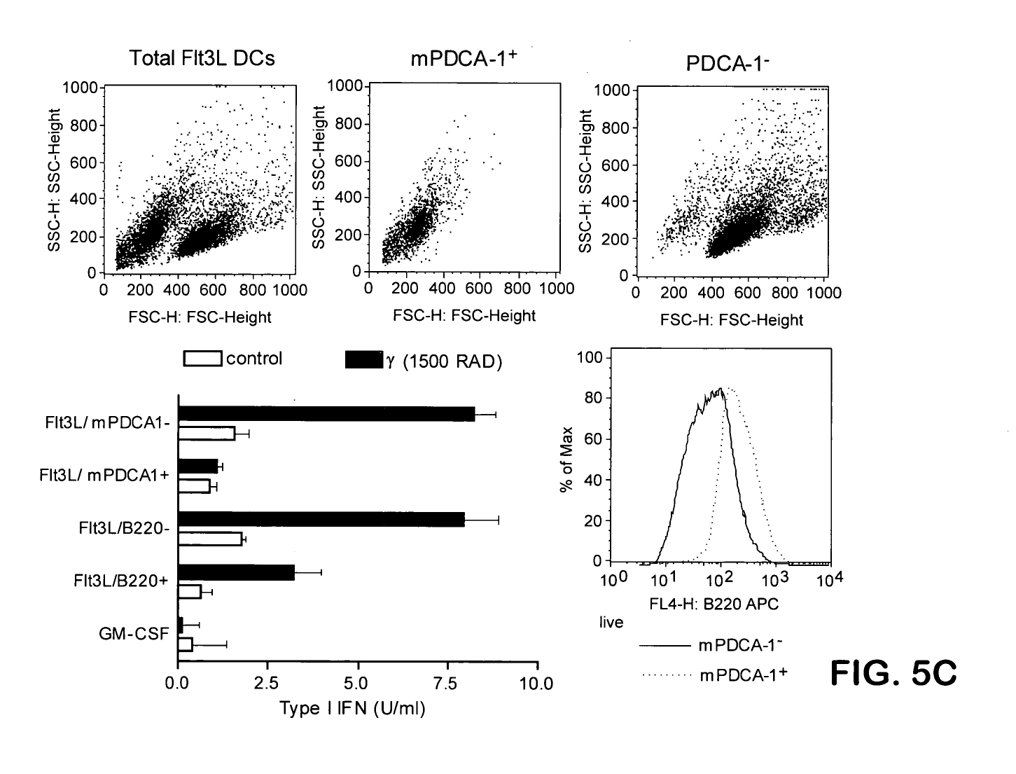

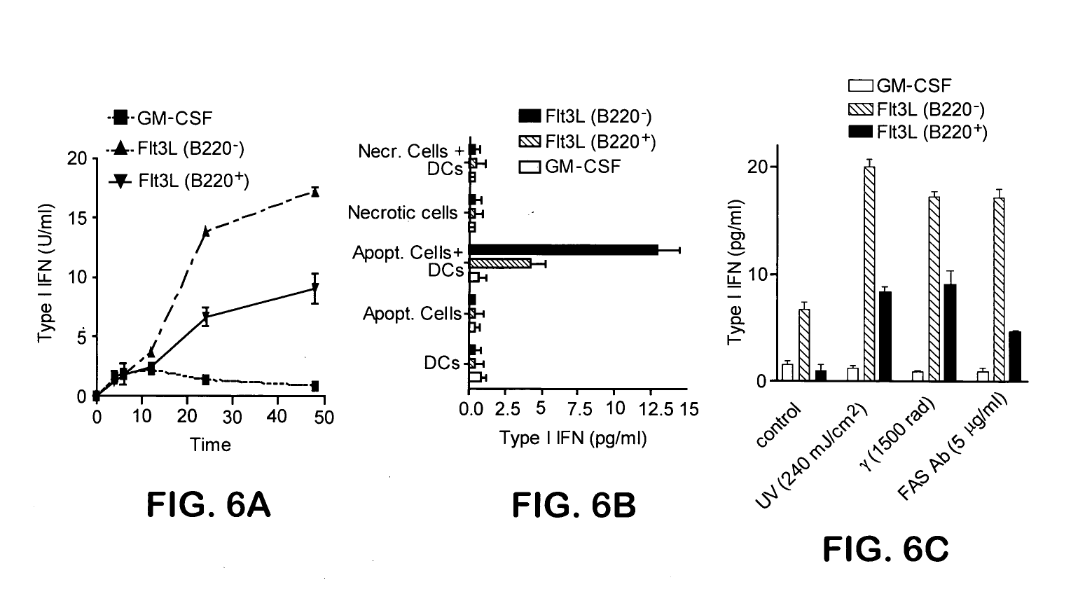

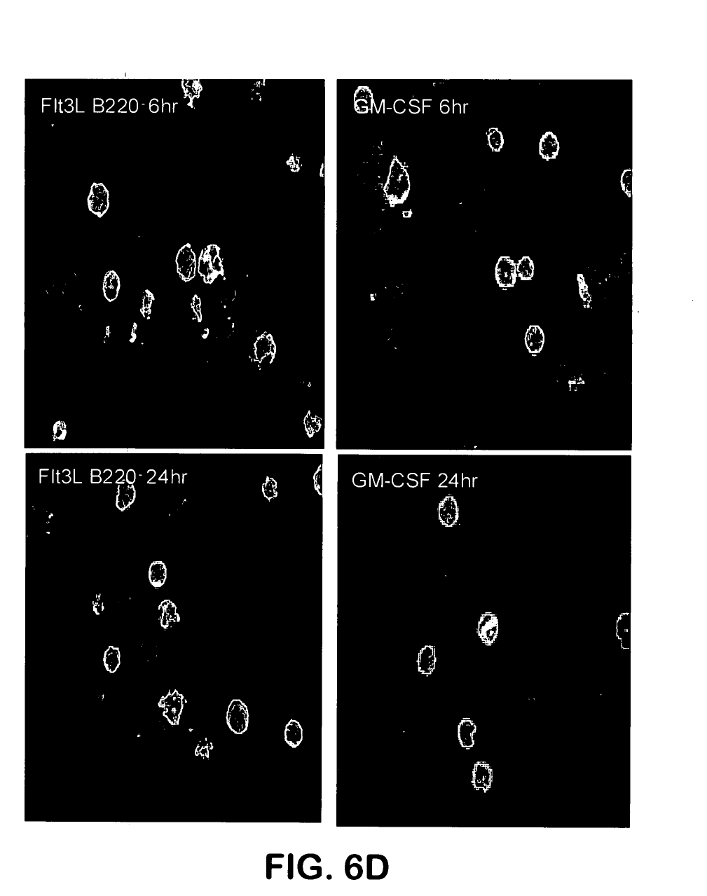

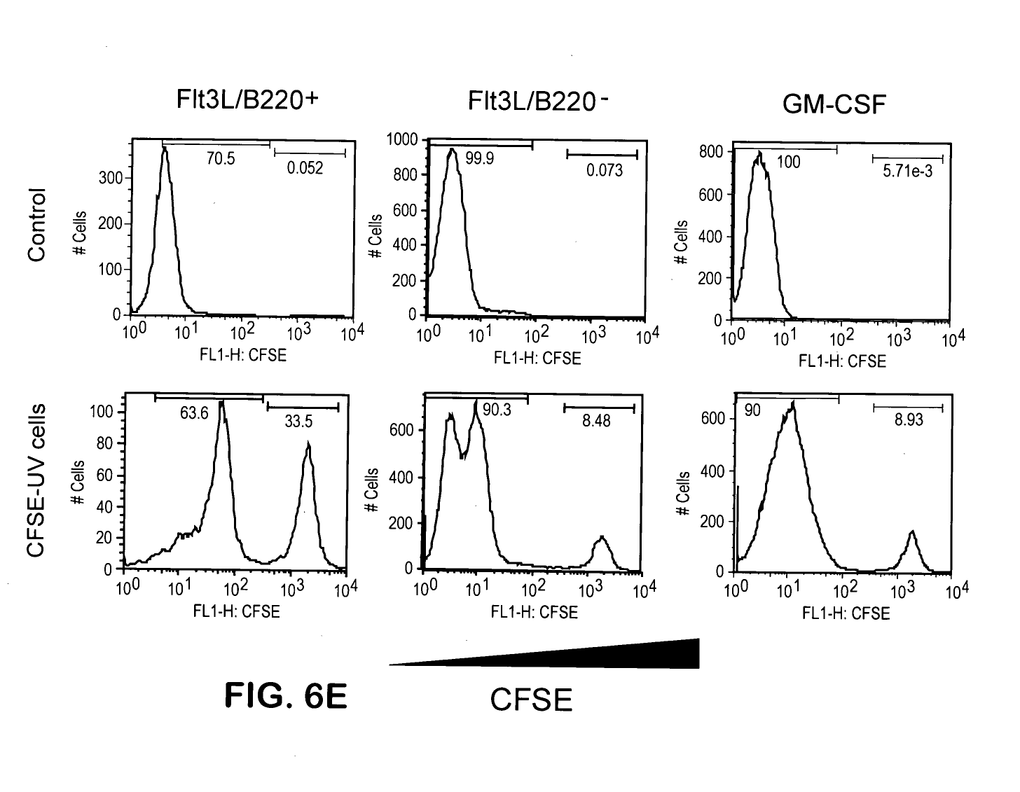

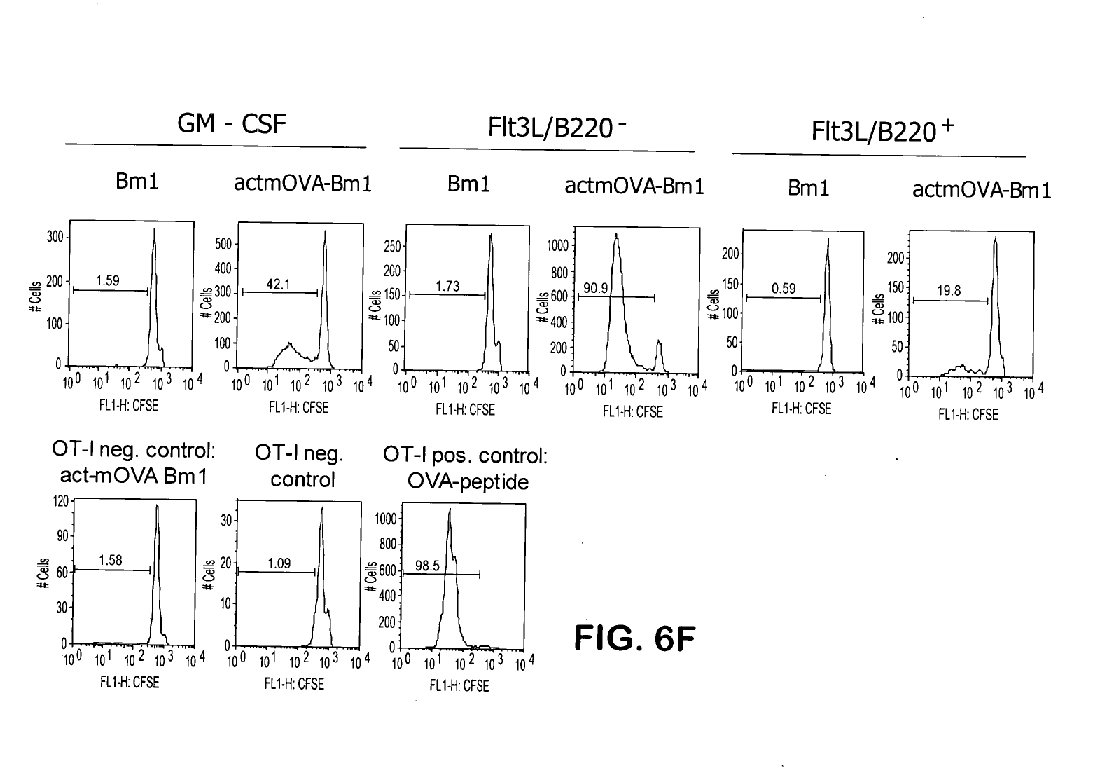

Dendritic cells (DC) and macrophages have previously been implicated as the major cell types involved in uptake of apoptotic cells, and DCs are potent inducers of both CD4⁺ and CD8⁺ T cell responses. Either conventional myeloid (MDC) or plasmacytoid DCs (PDC) might be responsible for recognition of apoptotic cells and subsequent type I IFN production. GM-CSF-derived DCs and B220⁺ and B220⁻ Flt3 Ligand (FIt3L)-derived DCs were generated from bone marrow of C57BL/6 mice. The cells were examined for expression of myeloid- and lymphoid-specific markers (FIG. 5a) and for respones to CpG and LPS (figure Sb). These cells were then exposed to apoptotic syngeneic splenocytes and monitored type I IFN production over 48 hrs. GM-CSF-derived DCs did not produce significant levels of type I IFN, while B220⁻ and, to a lesser extent, B220⁺ FIt3L-derived DCs were efficient type I IFN producers when exposed to apoptotic cells (FIG. 6a). The response became apparent after 24 hrs, consistent with the observations when whole spleens were used as responders, and was independent of the apoptotic stimulus used. To specifically address the possibility that PDCs were able to produce type I IFN in response to apoptotic cells, PDCs were separated from non-PDCs using a specific antibody recognizing mouse plasmacytoid dendritic cell antigen-1 (mPDCA-1). While FIt3L-derived mPDCA1⁻ DCs showed a similar type I IFN production as observed for B220⁻ DC, PDCA⁺ cells were unable to respond to apoptotic cells, suggesting the type I IFN response depended upon FIt3L-derived mPDCA1⁻/B220⁻ DCs.

In addition to the poor ability to induce CTL responses, necrotic cells were also unable to induce type I IFN in any of the DC subtypes (FIG. 6b). Apoptotic cells obtained after UV-irradiation (240 mJ/cm²), γ-irradiation (1500 rad)- or exposure to Fas-activating antibody (5 μg/ml) induced similar levels of type I IFN production (FIG. 6c).

### EXAMPLE 5

Flt3L-Derived B220⁻ DCs Acquire Apoptotic Cell-Derived Antigen via “Nibbling” and are Highly Efficient in Cross-Priming

UV-treated splenocytes were labeled with a fluorescent marker (CFSE) and exposed to FIt3L-derived B220⁺ and B220⁻ DCs as well as GM-CSF-derived DCs for 24 hrs. While GM-CSF-derived DCs were able to engulf apoptotic cells (FIGS. 5d, 6d), Flt3L-derived B220⁻ (FIG. 6d) and B220⁺DCs were not. However, both B220⁻ and B220⁺ Flt3L-derived DCs acquired apoptotic material via a process that resembled “nibbling”, which was observable after 24 hr of co-incubation (FIGS. 5e, 6d). The uptake of apoptotic material could be quantitated by FACS (FIG. 6e).

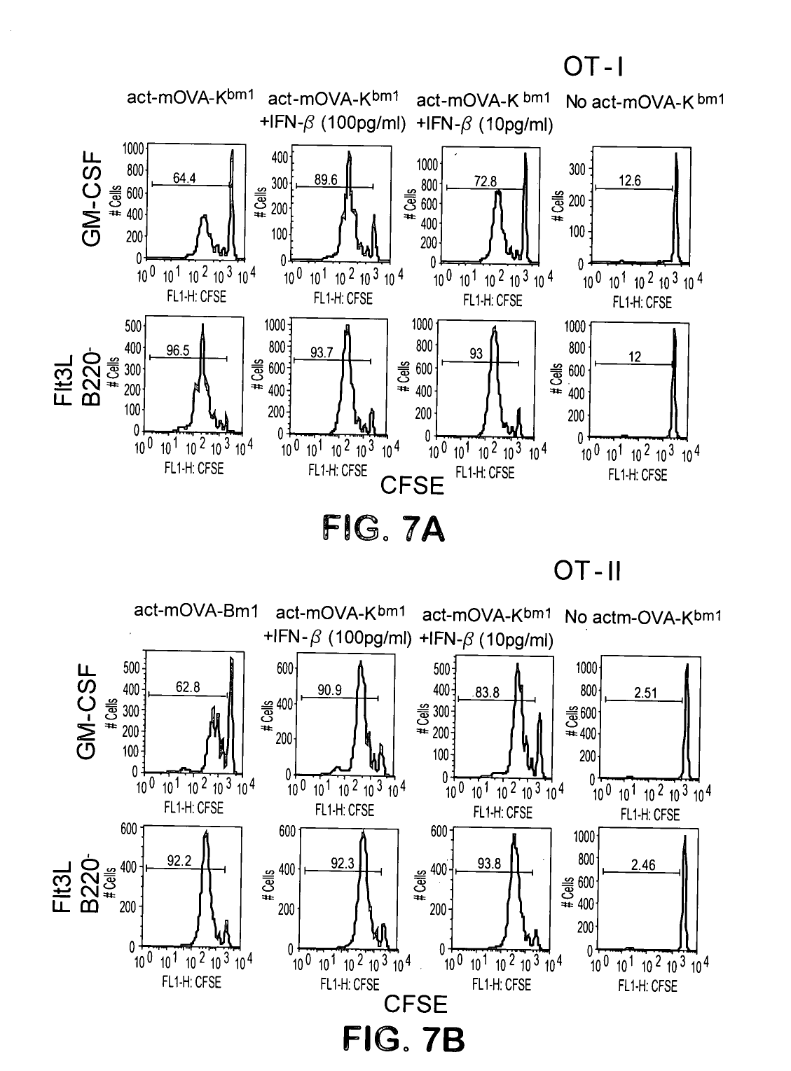

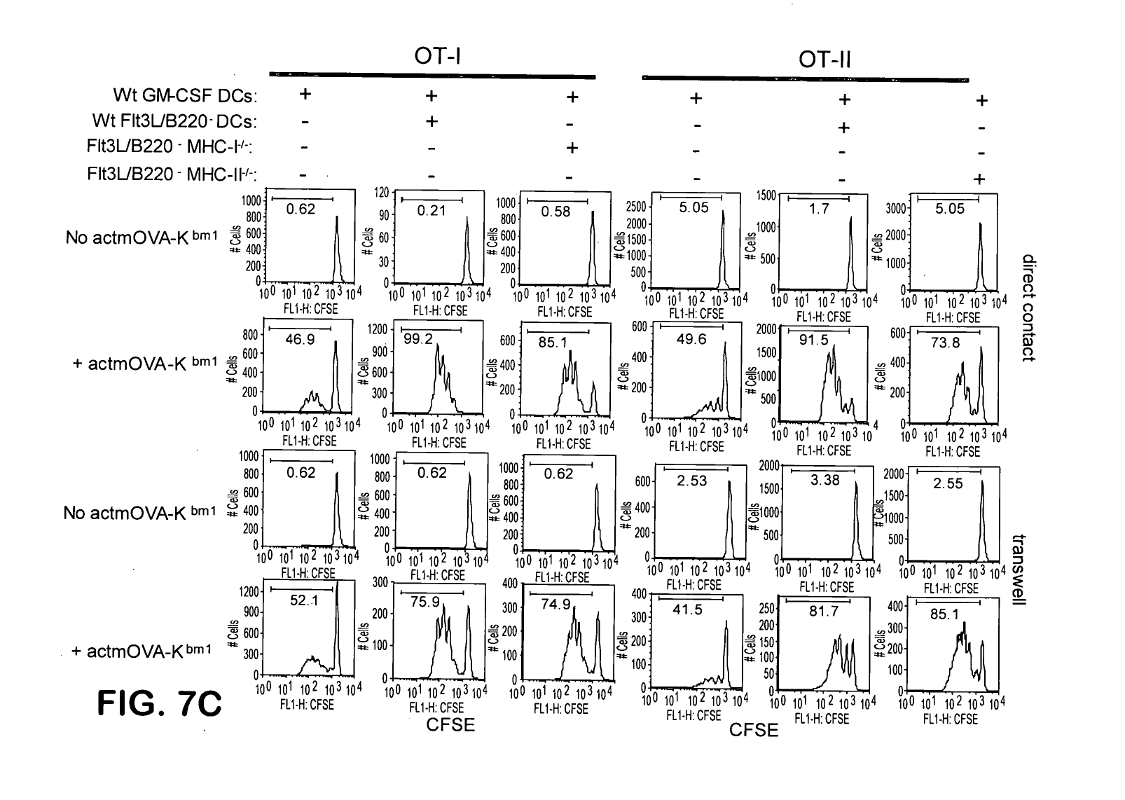

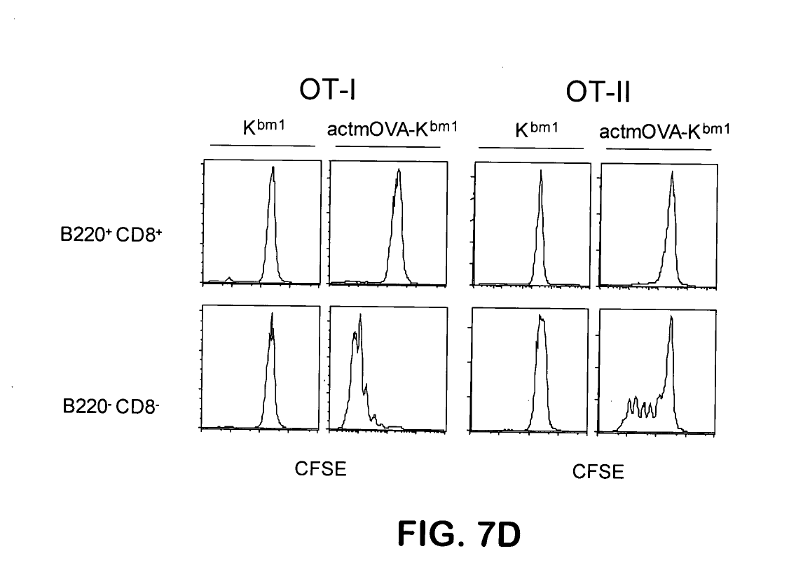

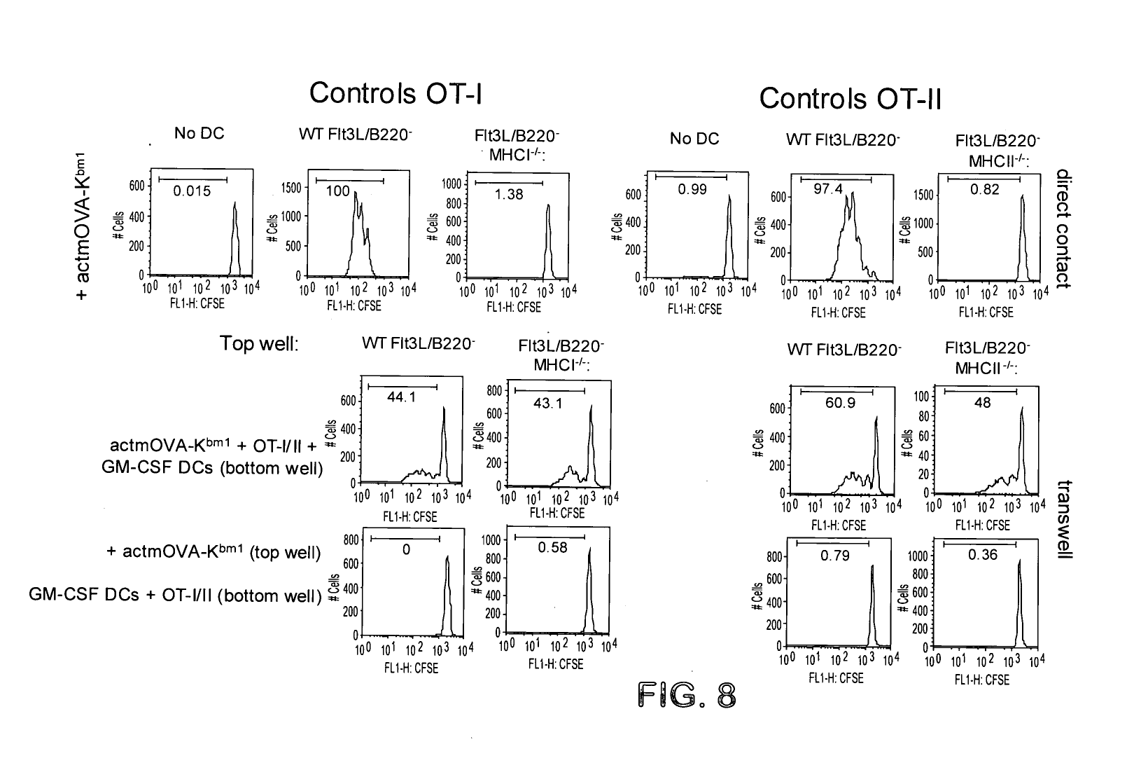

The ability of each DC-subtype to cross-prime OT-I CD8⁺ T-cells was examined after 24 hr pre-exposure to apoptotic cells that carried the act-mOVA transgene on a C57BL/6 K^{bm-1} background (thus excluding direct activation of OT-I cells via act-mOVA cells). B220⁻ FIt3L-derived DCs were highly efficient in cross-priming OT-I cells, whereas both GM-CSF- and B220⁺ Flt3L-derived DCs induced little or no cross-priming (FIG. 6f). The same results were observed for priming of OT-II CD4 T cells. Co-incubation of GM-CSF-derived DCs with apoptotic cells in the presence of type I IFN (FIG. 7a, 7b) or B220⁻ Flt3L-derived DCs that were physically separated through transwell systems (FIG. 7c) restored the ability of these DCs to (cross-) prime OT-I and OT-II cells. In the latter case, cross-priming of OT-I cells by GM-CSF-derived DCs was only observed when B220⁻ FIt3L-derived DCs were in direct contact with apoptotic cells (FIG. 8), suggesting that the apoptotic cell-derived IFN-inducing factor is unable to pass the membrane and therefore does not represent a soluble factor released from apoptotic cells.

FIG. 5 shows that (a) flow cytometric and (b) functional characterization of B220⁺ and B220⁻ FIt3L- and GM-CSF-derived dendritic cells from bone marrow. (c) mPDCA1⁻/B220⁻, but not mPDCA1⁺/B220⁺ Flt3L-derived, DCs are able to respond to γ-irradiated cells. (d,e) Interaction between GM-CSF (d) and FIt3L-derived (e) DCs with CFSE-labeled apoptotic cells after 24 hr co-incubation. Movies represent 3-dimensional images from slices through the cells.

FIG. 6 shows that FIt3L- and GM-CSF-derived BMDCs respond differently to apoptotic cells. (a) Type I IFN production by GM-CSF, and FIt3L-derived BMDCs during a 48hr incubation period with UV-treated wildtype splenocytes. (b) Type I IFN production in different BMDC subsets exposed to γ-irradiated cells or cells subjected to multiple freeze/thawing cycles. (c) Induction of type I IFN by BMDCs exposed to cells treated with UV-radiation (120 mJ/cm²), γ-irradiation (1500 rad) or exposed to Fas-activating antibody (5 μg/ml). (d) Uptake of UV-treated splenocytes (CFSE-labeled) by B220⁻ FIt3L- and GM-CSF-derived BMDCs at 6 or 24 hrs of co-culture as analyzed by Laser Scanning Confocal Microscopy. Red stain represents mitochondrial activity as assessed by mitotracker red treatment. (e) FACS analysis of FIt3L-derived B220⁻ or B220⁺ DCs after 24 hr incubation with or without CFSE-labeled apoptotic (UV-treated) splenocytes. (f) Proliferation of CFSE-labeled OT-I cells by GM-CSF and FIt3L-derived BMDCs, pre-exposed to apoptotic act-mOVA-K^{bm1} cells for 24 hr. Proliferation was determined by FACS analysis after an additional 72 hrs incubation

FIG. 7 shows that Flt3L-derived B220⁻ DCs efficiently (cross)-prime CD8⁺ and CD4⁺ T cells and can serve as bystander cells. FIt3L- and GM-CSF-derived DCs were exposed to UV-treated (240 mJ/cm²) act-mOVA-K^{bm1} splenocytes 24 hr in the presence or absence of IFN-β, prior to the addition of CFSE labeled (a) OT-I CD8⁺ T cells or (b) OT-II CD4⁺ T cells. Proliferation was determined after 72 hrs incubation using FACS analysis. (c) Rescue of GM-CSF-derived (cross-) priming of OT-I CD8⁺ or OT-II CD4⁺ T cells when cocultured with Flt3L-derived B220⁻ BMDCs on a wildtype, MHC-I^{-/-} or MHC-II^{-/-} background, either cultured in direct or indirect contact using a transwell system. In the latter, both top and bottom wells contain apoptotic act-mOVA cells, with top wells containing Flt3L-derived B220⁻ BMDCs and bottom wells containing GM-CSF-derived DCs and OT-I CD8 or OT-II CD4 T cells. Control experiments include apoptotic act-mOVA cells in the bottom or top well only (IS-3). (d) Proliferation of CFSE-labeled CD8⁺ OT-I and CD4⁺ OT-II T cells by MHC-II⁺/CD11c⁺/CD11b⁻/CD8⁺/B220⁺ and MHC-II⁺/CD11c⁺/CD11b⁻/CD8⁻/B220⁻ DC subsets directly isolated from spleen and cultured in the presence or absence of γ-irradiated act-mOVA-K^{bm-1} cells.

FIG. 8 shows that control incubations for (cross-)priming of OT I/II cells by the different DC subsets. Note that FIt3L-derived B220⁻ DCs cannot serve as bystander cells when direct contact with apoptotic cells is omitted by using transwell membrane inserts.

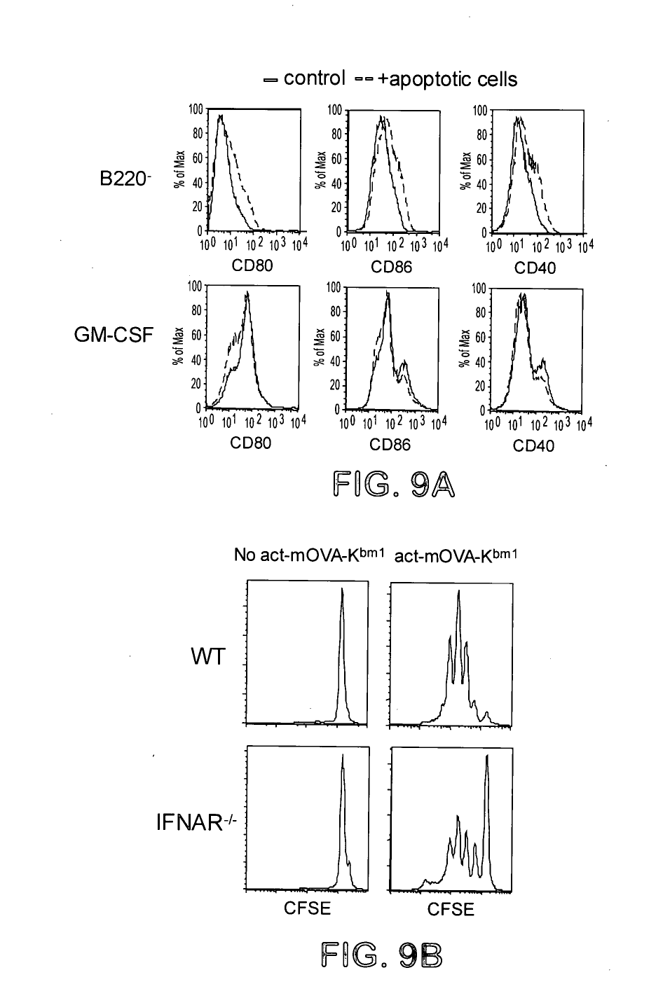

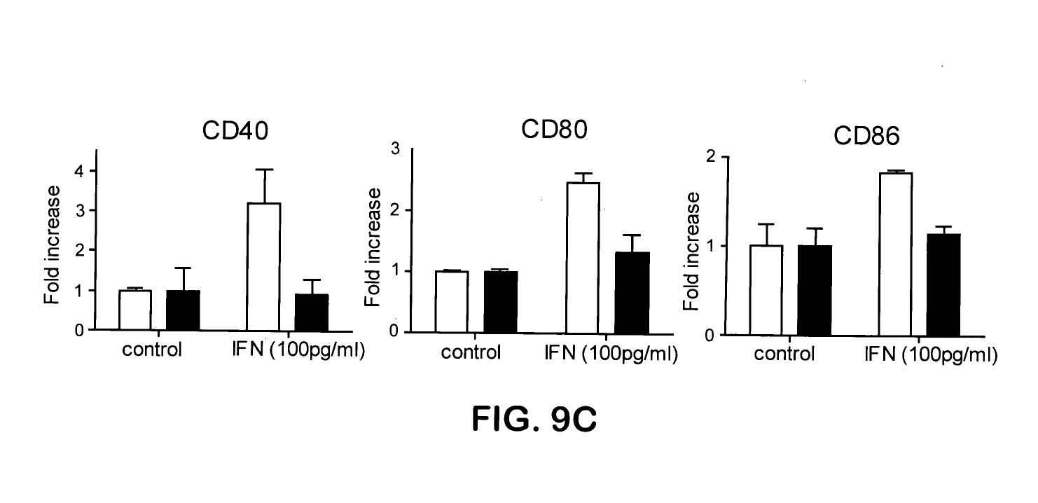

Type I IFN has emerged as an important mediator for CTL responses and costimulatory molecule expression. Hoebe el al., Nat. Immunol. 4: 1223-1229, 2003; Le Bon et al., Curr. Opin. Immunol. 14: 432-436, 2002; Montoya et al., Blood 99: 3263-3271, 2002. Costimulatory molecule expression on GM-CSF and B220⁻ FIt3L-treated bone marrow-derived DCs were tested after exposure to apoptotic cells. While CD40, CD80 and CD86 expression were increased on B220⁻ FIt3L-derived DCs, expression was unchanged or decreased on GM-CSF-derived DCs (FIG. 9a). Furthermore, FIt3L-derived DCs from Ifnar^{-/-} mice showed limited ability to activate OT-I cells in vitro (FIG. 9b), suggesting that type I IFN acts on DCs in an autocrine/paracrine manner independent of T cells. Finally, addition of type I IFN directly induced costimulatory molecule expression on FIt3L-derived B220⁻ DCs (FIG. 9c), confirming the important role of type I IFN in priming DCs ultimately leading to efficient T cell activation.

The cross-priming ability of various DC subsets were confirmed ex vivo by isolating phenotypically-identical dendritic cells directly from the spleen. While CD11c⁺/MHC-II⁺/CD11b⁻/CD8⁺/B220⁺ cells were unable to stimulate cross-priming after exposure to apoptotic act-mOVA cells, CD11c⁺/MHC-II⁺/CD11b⁻/CD8⁻/B220⁻ were highly efficient in activating CD8⁺ OT-1 cells and, to a lesser extent, CD4⁺ OT-II cells (FIG. 7d), confirming previous results obtained using bone-marrow-derived Flt3L-treated DCs. Together, these findings suggest CD11c⁺/MHC-II⁺/CD11b⁻/CD8⁻/B220⁻ DCs, acting either directly or as a source of type I IFN, may be key inducers of CTL responses stimulated by apoptotic cells.

FIG. 9 shows that (a) expression of CD40, CD80 and CD86 costimulatory molecules on GM-CSF and FIt3L-derived B220⁻ BMDCs after 24 hr incubation with or without apoptotic cells. (b) Cross-priming capacity of wildtype (Wt) and Ifnar^{-/-} FIt3L-derived B220⁻ BMDCs after 24 hr exposure to γ-irradiated act-mOVA-K^{bm1} cells. (c) Upregulation of costimulatory molecule expression in wildtype (white bar) and Ifnar^{-/-} (black bar) FIt3L-derived B220⁻ BMDCs after 24 hr incubation with IFN-β (100/pg/ml). Values represent mean duplicate values of 3 independent experiments.

### EXAMPLE 6

Involvement of Unc93b1 and Cd36 in CD4 and CD8 Responses

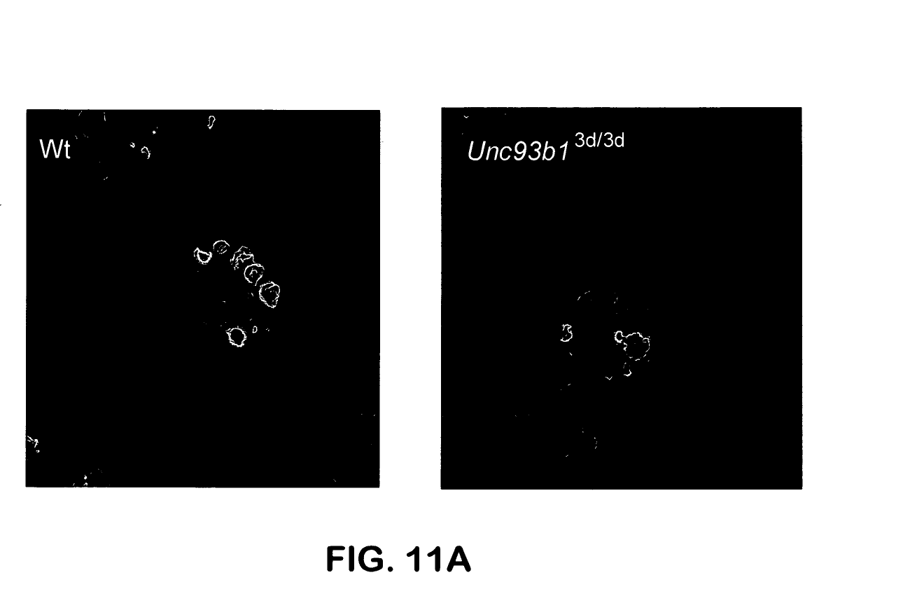

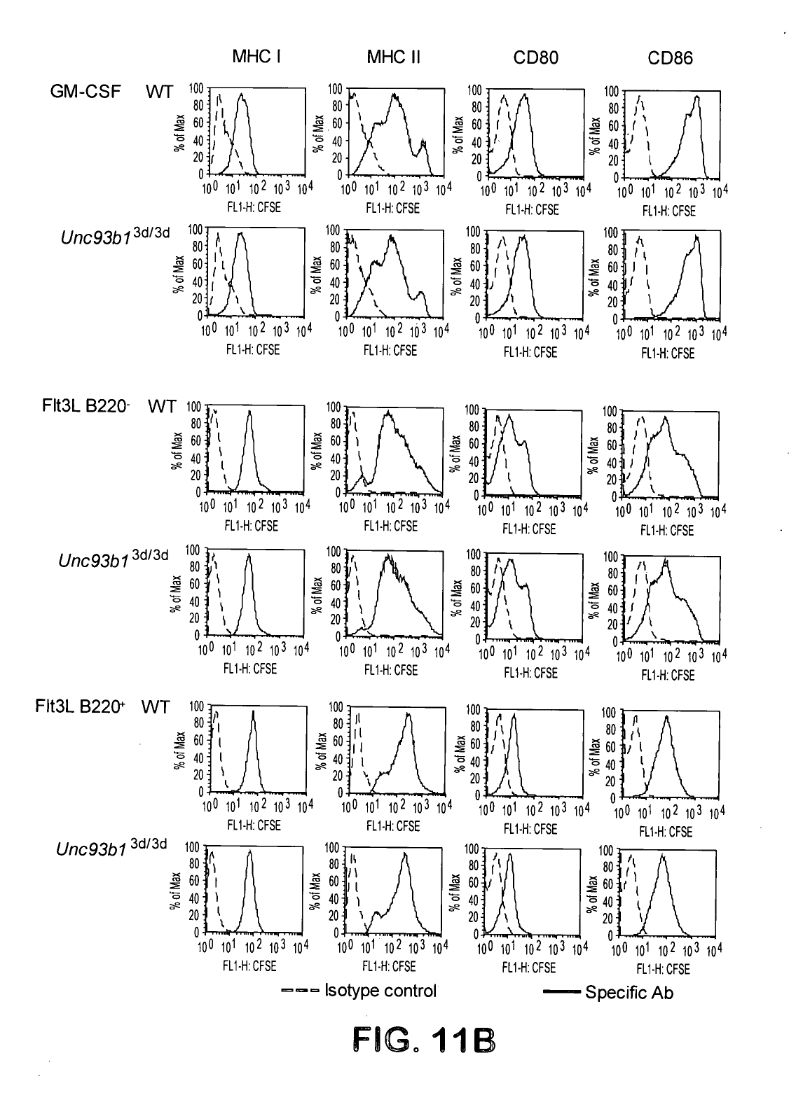

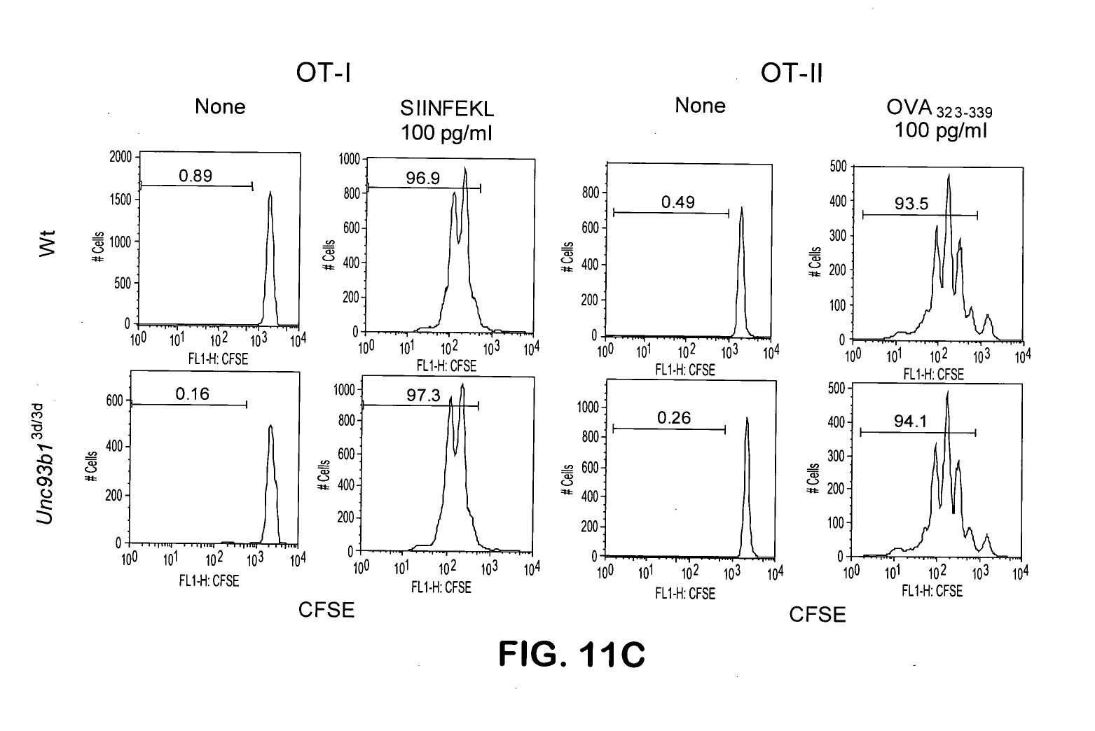

Among numerous ENU-induced mutations known to affect innate immune responses, two exhibited significant inhibitory effects on adaptive responses induced by apoptotic cells. Oblivious a nonsense allele of Cd36 known to impair sensing of microbial diacylglycerides, abrogated CD4 responses, but not CD8 responses, in homozygous mutant mice (FIGS. 10a-10c). Hoebe et al., Nature 433: 523-527, 2005. Strikingly, the mutation had no effect on type I IFN production (FIG. 10d). The 3dallele of Unc93b1, which encodes UNC-93B, a 12-spanning ER membrane protein required for responses to TLR3, 7 and 9 ligands, abrogated both CD8⁺ and CD4⁺ responses in homozygotes (FIGS. 10b, 10c). Tabeta et al., Nat. Immunol. in press, 2006. Again, type I IFN production was unaffected (FIG. 10d). Neither mutation affected the uptake of apoptotic cells by GM-CSF- or Flt3L-derived DCs (FIG. 11a) or decreased expression of MHC class I or class II antigens on the cell surface (FIG. 11b). However, cross-priming of OT-I CD8 cells was abrogated, and reduced priming of OT-II CD4⁺ cells was observed in Unc93b1^{3d/3d} mice (FIG. 10e). Additional studies using antibodies specific for the OVA-MHC I complex showed that Unc93b1^{3d/3d} mice were defective in MHC class I antigen presentation when exposed to either apoptotic act-mOVA cells (FIGS. 10f, 10g) or soluble ovalbumin. Tabeta et al., Nat. Immunol. in press, 2006. Finally, Unc93b1^{3d/3d} DCs directly loaded with low concentrations of OVA peptides were able to (cross-) prime OT-I or OT-II cells (FIG. 11c). Together, these findings suggest that the defect observed in Unc93b1^{3d/3d} mice is related to defective antigen processing rather than recognition or uptake of apoptotic cells.

FIG. 10 shows that CD36 and UNC-93B play a role in apoptotic cell-induced immune responses. Wildtype (Wt), Cd₃₆^{obl/obl} and Unc93b1^{3d/3d} mice were immunized with UV treated act-mOVA splenocytes and analyzed after subsequent secondary expansion in vitro for the frequency of CD8-specific IFN-γ-positive cells in response to SIINFEKL (*=P<0.001 between actm-OVA immunized Wt and Unc93b1^{3d/3d} mice) (a), as well as their ability to kill EL4 target cells (#=P<0.0001 between C57BL/6 and Cd36^{obl/obl} or Unc93b^{3d/3d} proflieration) (b). (N=4 mice). c) CD4-specific proliferation in wildtype, Cd36^{obl/obl} and Unc93b1^{3d/3d} mice immunized with apoptotic act-mOVA cells. (d) Induction of type I IFN in wildtype, Cd₃₆^{obl/obl} and Unc93b1^{3d/3d} splenocytes after 24 hr exposure to untreated or UV-treated cells (120 or 240 mJ/cm²). Values represent mean values±SEM of 3 independent experiments. (e) Activation of CFSE-labeled OT-I and OT-II T cells by B220⁻ FIt3L-derived DCs from wildtype and Unc93b1^{3d/3d} mutant mice pre-exposed to apoptotic act-mOVA-K^{bm-1} cells for 24 hr. Proliferation was determined by FACS analysis after an additional 72 hrs incubation. (f,g) GM-CSF and FIt3L-treated B220⁻ BMDCs from Unc93b1^{3d/3d} mice show abrogated MHC/SIINFEKL-complex formation. DCs were incubated for 24 hrs with γ-irradiated act-mOVA-K^{bm-1} splenocytes. Formation of MHC class I/SIINFEKL-complex was determined using a specific antibody, as described in material and methods. Values are mean values±SEM from triplicate values and graph is representative of 3 independent experiments.

FIG. 11 shows that DC subsets derived from Unc93b1^{3d/3d} mutant mice show normal surface expression of MHC class I/II and costimulatory molecule expression (a) as well as normal uptake of apoptotic cells (b). In addition, exposure of Flt3L-derived B220⁻ DCs from Unc93b1^{3d/3d} to extracellular OVA-specific MHC class I and II peptides (100 pg/ml) results in normal (cross-) priming of OT-I or OT-II cells as compared to wildtype (Wt) DCs (c).

### EXAMPLE 7

Apoptotic Cells Trigger an Innate and Subsequent Adaptive Immune Response that is Independent of TLR Signaling

TLRs mediate most phenomena associated with microbial infections, including the well-known immunoadjuvant effect of infection. However, mice that are incapable of TLR signal transduction have grossly normal lymphoid tissues, produce IgG, and are capable of allograft rejection, indicating that TLRs are not obligatory for adaptive immune responses per se. The present study shows that apoptotic cells trigger an innate and subsequent adaptive immune response that is independent of TLR signaling. Of key importance in this pathway is the recognition of apoptotic cells by FIt3L-derived B220⁻ DCs and their concomitant production of type I IFN. Type I IFN is an important mediator of adjuvanticity via TLR-dependent pathways, and, as shown here, is also a central mediator of adjuvanticity elicited by apoptotic cells, but via a TLR-independent pathway. Hoebe et al., Nat. Immunol. 4: 1223-1229, 2003; Le Bon et al., Nat. Immunol. 4: 1009-1015, 2003. PDCs are thought to be specialized type I IFN-producing cells during viral infections, yet clearly myeloid or conventional DCs can serve a similar function utilizing alternative signaling pathways. Asselin-Paturel et al., J. Exp. Med. 202: 461-465, 2005; Kato et al., Immunity 23: 19-28, 2005. The current data suggest that production of type I IFN induced by cell death depends on direct recognition of dying cells by B220⁻/mPDCA-1⁻DCs and not by pDCs, whereas the removal of apoptotic cells primarily depends upon GM-CSF-derived DCs. In addition, Flt3L-derived B220⁻ DCs are capable of “sampling” apoptotic cells through “nibbling” and are highly efficient in cross-priming CD8⁺ T-cells.

Several triggers of apoptosis are capable of initiating events that lead to a strong adaptive immune response. CTL responses were elicited with act-mOVA cells rendered apoptotic via UV- and γ-irradiation, and also after treatment with Fas-activating antibodies. The latter effect implicates an apoptotic pathway that is intimately involved in immune responses. Fas receptor is expressed on many cell types in different organs and induces apoptosis via a caspase-dependent mechanism. Barnhart et al., Semin. Immunol. 15: 185-193, 2003. The engagement of Fas with its cognate ligand (FasL) is used by virtually every effector immune cell that is activated to mediate cytotoxicity towards infected cells or tumor cells. Dennert, Crit Rev. Immunol. 22: 1-11, 2002; Smyth et al., Mol. Immunol. 42: 501-510, 2005. In addition, many viruses or bacteria can directly induce apoptosis that can lead to inflammatory responses. The TLR-independent pathway therefore detects potential intracellular infections and also aids the development of CTL responses when cell death occurs via NK cells or T-cell mediated cytotoxicity. Recent studies report that both dsRNA and mammalian DNA induce type I IFN via TLR-independent pathways. Ishii et al., Nat. Immunol. 7: 40-48, 2006; Kato et al., Immunity 23: 19-28, 2005; Okabe et al., J. Exp. Med. 202: 1333-1339, 2005; Yoneyama et al., Nat. Immunol. 5: 730-737, 2004. However, these pathways are present in conventional myeloid DCs, which as shown herein, are deficient in type I IFN production upon exposure to apoptotic cells. Furthermore no effect on apoptotic cell mediated type I IFN production in the presence of DNase I and/or RNase was found. These observations suggest that the production of type I IFN is independent of DNA or RNA, yet the possibility that these nucleotide structures enter the cytosol directly without exposure to the extracellular environment cannot be excluded.

Although not all of the molecular components of the death-induced immunoadjuvant pathway are known, the present study has shown that CD36 and UNC-93B both contribute to CD4⁺ T cell priming, while UNC-93B is required for cross priming of CD8⁺ T cells. Neither protein is required for type I IFN production, hence, neither is solely required for sensing apoptotic cells. In the intact Cd36^{obl} environment, MHC class II priming, measured by OT-II proliferation, occurs normally in response to apoptotic cells. This suggests that CD36 fulfills a role related to the primary expansion of antigen-specific CD4⁺ T cells, but is not required for MHC class II primingper se. The role of CD36 as a sensor of diacylglycerides may imply that a lipid component of apoptotic cells supports this expansion, and that CD36 mediates this effect through a TLR-independent process. Hoebe et al., Nature 433: 523-527, 2005. As to UNC-93B, its diverse functions in immunity include support of signaling via endosomal TLRs (3, 7, 8, and 9) as well as MHC antigen presentation leading to (cross-) priming of CD8⁺ and CD4⁺ T cells. Tabeta et al., Nat. Immunol. in press, 2006. The exact mechanism whereby it exerts these effects has yet to be deciphered.

Auto-amplification loops, based upon the binding of pre-existing immune complexes either to B-cells (via TLR-dependent but also TLR-independent pathways) or to PDCs (via FcγRIIa) have been proposed as a fundamental mechanism in systemic lupus erythrematosus (SLE), the prototypic systemic autoimmune disease. Boule et al., J. Exp. Med. 199: 1631-1640, 2004; Lau et al., J. Exp. Med. 202: 1171-1177, 2005; Leadbetter et al., Nature 416: 603-607, 2002; Martin el al.; J. Exp. Med. 202: 1465-1469, 2005; Vollmer et al., J. Exp. Med. 202: 1575-1585, 2005; Bave et al., J. Immunol. 171: 3296-3302, 2003; Lovgren et al., Arthritis Rheum. 50: 1861-1872, 2004. In the former case, the adaptive immune response was dependent upon TLR activation, while the latter was accompanied by type I IFN production. The death-driven immunoadjuvant pathway described here may also amplify the autoimmune response in SLE, a view consistent with defects in removal of apoptotic bodies, the existence of a type I IFN “signature” in blood cells and afflicted tissues of patients, and the requirement for type I IFN in some spontaneous models of this disease. Herrmann et al., Arthritis Rheum. 41: 1241-1250, 1998; Theofilopoulos et al., Annu. Rev. Immunol. 23: 307-335, 2005. As such, it would represent a proximal autoimmune pathway that could function independent of pre-existing autoantibodies. Mutations that alter the normal pattern of cell death, impair the removal of apoptotic cells, or enhance immune perception of apoptotic cells, might be primary contributors to autoimmunity. Moreover, interruption of the apoptotic cell-induced immune response might have a substantial mitigating effect in autoimmune disease.

### EXAMPLE 8

Materials and Methods

Mice and materials. All experiments were performed according to the US National Institutes of Health guidelines. C57BL/6 wildtype, CD36^{obl/obl}, unc93b1^{3d/3d} mutant, OT-I, OT-II and MyD88^{-/-}; Trif^{lps2/lps2} double deficient mice were bred on a C57BL/6 background and housed in the Scripps Research Institute Vivarium. All knockout mice used in the experiments were transferred on a C57BL/6 background after repeated backcrossing to C57BL/6 mice (at least 6 times). IFNAR knockout mice were kindly provided by Dr. Jonathan Sprent (The Scripps Research Institute, La Jolla) and act-mOVA transgenic mice were a kind gift from Dr. Mark Jenkins (University of Minnesota Medical School, Minneapolis, USA) and were bred onto a K^{bml} background.

Escherichia coli RE595 LPS was obtained from Alexis (San Diego, Calif., USA) and dsRNA (poly (I:C)) was obtained from Amersham Pharmacia Biotech (Piscataway, N.J., USA). Resiquimod was a kind gift from Novartis (Basel, Switzerland). PAM₃CSK₄ and Malp2 were obtained from EMC microcollections GmbH (Tubingen, Germany). Phosphorothioate-stabilized CpG oligodeoxynucleotide (CpG ODN) 5′-TCC-ATG-ACG-TTC-CTG-ATG-CT-3′ was obtained from Integrated DNA Technologies (Coralville, Iowa USA). Zymosan A was obtained from Sigma (St Louis, Mo., USA). Fluorescent-labeled antibodies directed against IFN-γ, CD11b, CD11c, MHC-II, B220, CD4 and CD8 were obtained from eBioscience (San Diego, Calif., USA) and microbeads for negative selection of CD4 and CD8 T cell and positive selection of B220, mPDCA-1 and CD11c were obtained from Miltenyi Biotec (Auburn, Calif., USA). Human Flt3 Ligand was purchased from Preprotech, Inc. (London, UK) and GM-CSF was from Becton Dickinson (Franklin Lakes, N.J. USA). CFSE and mitotracker Red CM-H₂XRos were obtained from Molecular Probes (Eugene, Oreg., USA). Ovalbumin-specific peptide for MHC class I (OVA₂₅₇₋₂₆₄: SIINFEKL) or MHC class II (OVA₃₂₃₋₃₃₉; ISQAVHAAHAEINEAGR) were purchased from A&A labs LLC, (San Diego, Calif., USA).

Immunization and Listeria monocytogenes challenge. The experiments involving immunizations and challenges with Listeria monocytogenes were performed as detailed elsewhere. Brockstedt et al., Nat. Med. 11: 853-860, 2005. Briefly, five 6-8 week old C57BL/6 male mice per group were subcutaneously injected with either 200 μl PBS, 200 μl irradiated splenocytes from wildtype or act-mOVA cells (1×10⁶ in 200 μl), or with 200 μl containing 1×10³ CFU of OVA expressing Listeria monocytogenes. Subsequently, mice were challenged with 1×10⁵ OVA expressing L. monocytogenes i.v. at day 8, 15 or 30 post immunization. After 3 days, spleens were isolated and the L. monocytogenes titer was determined after homogenization of total spleens in 5 ml dissociation buffer containing dH₂O (Gibco)+0.2% NP-40 (Sigma-Aldrich). Series of 10-fold dilutions in PBS were prepared and 100 μl of each dilution was plated onto bacterial culture plates containing Brain Heart Infusion agar+200 ug/mL streptomycin (Difco). After additional 24 hrs incubation at 37° C., the numbers of colonies were counted to determine the splenic titer.

Cytokine measurements. To measure cytokine responses in vitro, either total splenocytes (5×10⁵/well) or 5×10⁴ DCs/well in a 96 well-plate were cocultured with 5×10⁵ cells/well of UV- or γ-irradiated cells for 24 hrs. Control cultures included 10⁶ cells/well of apoptotic or non-treated cells. After 24 hrs, supernatants were collected and cytokines measured using bioassays or ELISA's. TNF-α production was measured using the L929 bioassay as previously described. Hoebe et al., J. Endotoxin. Res. 9: 250-255, 2003. The concentration of type I IFN was determined using a cell line containing an ISRE-responsive element luciferase reporter construct as described elsewhere. Jiang et al., Nat. Immunol. 6: 565-570, 2005. Production of IFN-γ was measured using a commercial ELISA (R&D Biosystems, Minneapolis, Mo., USA).

Measurement of cell death-induced CD4⁺ and CD8⁺ Tcell responses. Induction of cytolytic T lymphocyte responses via subcutaneous injection of apoptotic cells has been extensively described. Finberg et al., J. Immunol. 123: 1205-1209, 1979; Huang et al., Immunity 4: 349-355, 1996; Janssen et al., Nature 433[7029]: 88-93, 2005; Janssen et al., Nature 421: 852-856, 2003. Briefly, mice were immunized with act-m-OVA splenocytes that were either left untreated or rendered apoptotic through exposure to either γ-irradiation (1500 Rad), UV-irradiation (240 mJ/cm), or Fas-activating antibody (5 μg/ml). For treatment with Fas-activating antibody, splenocytes were incubated at 37° C. and 5% CO₂ for 2 hrs, followed by two thorough washings with medium and one wash with PBS. Mice were immunized subcutaneously with 10×10⁶ cells in PBS. Alternatively, mice were immunized with equivalent volume/numbers of necrotic cells obtained through multiple cycles (5 times) of freeze/thawing (−80° C.) in PBS. After 7 days, spleens were isolated and single cell suspensions were made in Iscove's modified Dulbecco's medium (IMDM) supplemented with 10% FCS, 2% penicillin/streptomycin (Invitrogen, San Diego, Calif.), and 50 μM β-mercaptoethanol (Sigma, St Louis, Mo., USA). The frequency of OVA-specific CD8⁺ T cells was determined after OVA₂₅₇₋₂₆₄-K^{b} tetramer staining or by intracellular IFN-γ staining upon 5 hr incubation with OVA₂₅₇₋₂₆₄ (5 μg/ml) in the presence of Brefeldin (Janssen et al., 2003 2005). To study the cytolytic capacity of the CD8⁺ T cells, splenocytes were cultured with irradiated MEC.B7.Sig-OVA cells (1:10) for 6 days. Restimulated splenocytes were evaluated by JAM test as previously described (Janssen et al., 2003, Schoenberger et al., 1999), using ³[H]thymidine-labeled EL-4 cells loaded with OVA₂₅₇₋₂₆₄ or control peptide. Specific killing was calculated as follows: (spontaneous c.p.m.—experimental c.p.m.)×100/spontaneous c.p.m. The proliferative capacity of OVA-specific CD4⁺ T cells was determined by standard lymphocyte stimulation assay. Briefly, splenocytes were cultured in 96 wells plates (2×10⁶ cells/well) with medium or increasing doses of OVA₃₂₃₋₃₃₉ for 72 hr, after which the cells were pulsed for 8 hr with ³[H]thymidine (0.1 μCi/well). Proliferation was calculated by dividing the incorporated c.p.m. of the OVA₃₂₃₋₃₃₉ stimulated cells by the c.p.m. of medium stimulated cells.

Generation of DC subsets and T cell purification. BM cells were isolated and cultured with FIt3L (200 ng/ml) or with GM-CSF (20 ng/ml) as previously described. Brasel et al., Blood 96: 3029-3039, 2000; Lutz et al., J. Immunol. Methods 223: 77-92, 1999. After 8 days, DCs were sorted in subpopulations based on their expression of B220 and CD11c or expression of mouse plasmacytoid dendritic cell antigen-1 (mPDCA-1) using MACS microbeads. DCs were thoroughly analyzed for surface marker expression and cytokine expression upon TLR ligation. Purity of all the different populations was >97%. OT-I (CD8+) and OT-II (CD4+) T cells were isolated by negative selection using T cell isolation kits (Miltenyi) and labeled with CFSE as described previously. Tabeta et al., Nat. Immunol. in press, 2006.

Determination of priming and cross-priming by different DC subsets. To determine the ability of DC subsets to prime purified CFSE-labeled OT-I or OT-II T cells, different DC subsets were cultured in IMDM (supplemented with 10% FCS, 2% penicillin/streptomycin, 50 μM β-mercaptoethanol) at a concentration of 1×10⁶ cells/well in the presence or absence of 3×10⁶ UV-treated act-mOVA cells (240 mJ/cm²) on a K^{bm1} background (to avoid direct presentation of act-mOVA cells to T cells). In addition, different DC subsets were cultured either in direct or indirect contact with apoptotic act-mOVA cells using 0.02 μm Anapore membrane Tissue culture inserts (Nalgene Nunc International, Rochester, N.Y.). Cells were incubated at 37° C., 5% CO₂, and after 24 hr incubation, either 2'10⁵ CFSE-labeled OT-I or OT-II T cells were added to each well. After an additional 72 hrs of incubation, CFSE intensity of OT-I or OT-II cells was determined by flow-cytometry analysis using specific fluorescent-labeled antibodies for CD4 or CD8 in combination with Vα2. In addition, OT-I or OT-II T cells were cultured alone or in the presence of act-mOVA/K^{bml} cells as negative controls, or with CD4- or CD8-specific OVA peptides as a positive control.

Isolation of DCs subsets from spleen. Spleens were treated with 2 mg/ml of collagenase D (Roche Diagnostics, Germany) in 10 mM Hepes-NaOH, 150 mM NaCl, 5 mM KCl, 1 mM MgCl₂, 1.8 mM CaCl₂ to obtain a single cell suspension. Subsequently, splenocytes were washed with PBS containing 0.5% FBS, 2% EDTA and CD11c⁺ cells were isolated using microbeads (Milteny Biotec, Auburn, Calif.). After 15 minutes incubation, cells were washed and separated using a LS MACS column. Subsequently, CD11c⁺ cells were labeled with fluorochrome-conjugated antibodies specific for MHC-II, CD11b, CD8, and B220 and directly separated by FACS sort. Both MHC-II⁺/CD11b⁻/CD8⁺/B220⁺ and MHC-II⁺/CD11b⁻/CD8⁻/B220⁻ subsets were collected and exposed to γ-irradiated act-mOVA-K^{bm-1} cells. OT-I and OT-II proliferation was assessed as described above.

Immuno-staining of SIINFEKL/K^{b} complex. Bone marrow derived GM-CSF and B220⁻ FIt3L-treated DCs from wildtype and 3 d mice were cocultured in the presence of γ-irradiated act-mOVA splenocytes (at various ratios. After 24 hrs, cells were stained with Alexa Fluor-750 coupled mAb 25-D1.16 specifically recognizing the SIINFEKL/K^{b} complex. Porgador et al., Immunity 6: 715-726, 1997.

Statistical analysis. All experiments were repeated at least three times and comparison between groups was performed using one way Analysis Of Variance (ANOVA).

All publications, patents, patent applications, polynucleotide and polypeptide sequence accession numbers and other documents cited herein are hereby incorporated by reference in their entirety and for all purposes to the same extent as if each of these documents were individually so denoted.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed:

1. A method for identifying an agent useful for modulating the immune system, comprising (i) contacting a test compound with an apoptotic cell-responding B220⁻ dendritic cell in the presence of an apoptotic cell, and (ii) detecting a change of a signaling activity of the apoptotic cell-responding B220⁻ dendritic cell relative to its signaling activity in the absence of the test compound; thereby identifying an agent useful for modulating the immune system.

2. The method of claim 1, wherein the apoptotic cell-responding B220⁻ dendritic cell is (i) a FIt3L-induced bone marrow-derived B220⁻ dendritic cell or (ii) a spleen derived CD11b⁻/CD11c⁺ B220⁻ dendritic cell.

3. The method of claim 1, wherein the apoptotic cell is a splenocyte treated with γ-irradiation, UV-irradiation or Fas-activating antibody.

4. The method of claim 1, wherein the signaling activity in the presence of the test compound is increased relative to the signaling activity in the absence of the test compound.

5. The method of claim 1, wherein the signaling activity in the presence of the test compound is decreased relative to the signaling activity in the absence of the test compound.

6. The method of claim 1, wherein the apoptotic cell-responding B220⁻ dendritic cell is contacted with the test compound prior to stimulation with the apoptotic cell.

7. The method of claim 1, wherein the apoptotic cell-responding B220⁻ dendritic cell is contacted with the test compound subsequent to stimulation with the apoptotic cell.

8. The method of claim 1, wherein the signaling activity is production of type I interferon by the dendritic cell upon stimulation with the apoptotic cell.

9. The method of claim 1, wherein the signaling activity is uptake by the dendritic cell of apoptotic material from the apoptotic cell.

10. The method of claim 9, wherein the apoptotic cell is a UV-treated splenocyte that is labeled with a fluorescent marker.

11. The method of claim 9, wherein uptake of apoptotic material by the dendritic cell is quantitated by FACS.

12. The method of claim 1, wherein the signaling activity is activation of a T cell proliferation.

13. The method of claim 12, wherein the T cell is a CD8⁺ T cell or a CD4⁺ T cell.

14. The method of claim 12, wherein the T cell is fluorescently labeled.

15. The method of claim 12, wherein proliferation of the T cell is examined by FACS.

16. A method for identifying a modulator of an antigen specific immune response, comprising (i) contacting a test compound with an apoptotic cell-responding B220⁻ dendritic cell in the presence of an apoptotic cell which comprises the antigen, and (ii) detecting a change of a signaling activity of the apoptotic cell-responding B220⁻ dendritic cell relative to its signaling activity in the absence of the test compound; thereby identifying a modulator of adaptive immune response specific for the antigen.

17. The method of claim 16, wherein the apoptotic cell-responding B220⁻ dendritic cell is (i) a Flt3L-induced bone marrow-derived B220⁻ dendritic cell or (ii) a spleen derived CD11b⁻/CD11c⁺B220⁻ dendritic cell.

18. The method of claim 16, wherein the apoptotic cell is a splenocyte treated with γ-irradiation, UV-irradiation or Fas-activating antibody.

19. The method of claim 16, wherein the antigen is a bacterial or viral antigen.

20. The method of claim 16, wherein the antigen is an autoantigen or an allogenic graft antigen.

21. The method of claim 16, wherein the compound enhances the signaling activity of the dendritic cell.

22. The method of claim 16, wherein the compound inhibits the signaling activity of the dendritic cell.

23. The method of claim 16, wherein the signaling activity is production of type I interferon by the dendritic cell upon stimulation with the apoptotic cell.

24. The method of claim 1, wherein the signaling activity is uptake by the dendritic cell of apoptotic material from the apoptotic cell.

25. The method of claim 1, wherein the signaling activity is activation of a T cell proliferation.

26. A method for identifying a modulator of innate immunity, comprising (i) contacting a test compound with an apoptotic cell-responding B220⁻ dendritic cell in the presence of an apoptotic cell, and (ii) detecting a change of a signaling activity of the apoptotic cell-responding B220⁻ dendritic cell relative to its signaling activity in the absence of the test compound; thereby identifying a modulator of innate immune response.

27. The method of claim 26, wherein the apoptotic cell-responding B220⁻ dendritic cell is (i) a FIt3L-induced bone marrow-derived B220⁻ dendritic cell or (ii) a spleen derived CD11b⁻/CD11c⁺B220⁻ dendritic cell.

28. The method of claim 26, wherein the apoptotic cell is a splenocyte treated with γ-irradiation, UV-irradiation or Fas-activating antibody.

29. The method of claim 26, wherein the compound enhances the signaling activity of the dendritic cell.

30. The method of claim 26, wherein the compound inhibits the signaling activity of the dendritic cell.

31. The method of claim 26, wherein the signaling activity is production of type I interferon by the dendritic cell upon stimulation with the apoptotic cell.

32. The method of claim 26, wherein the signaling activity is uptake by the dendritic cell of apoptotic material from the apoptotic cell.

33. The method of claim 26, wherein the signaling activity is activation of a T cell proliferation.
